(19) United States
(12) Patent Application Publication
(10) Pub. No.: US 2025/0295760 A1
(43) Pub. Date: Sep. 25, 2025

(54) MODIFIED CORONAVIRUS S PROTEIN

(71) Applicant: ARAMIS BIOTECHNOLOGIES INC., Quebec City (CA)

(72) Inventors: Marc-André D'Aoust, Quebec (CA); Pierre-Olivier Lavoie, Québec (CA); Marie-Eve Ouellet, Quebec City (CA); Mikael Bedard, Quebec City (CA)

(21) Appl. No.: 18/862,407

(22) PCT Filed: May 1, 2023
(86) PCT No.: PCT/CA2023/050590

§ 371 Date: Nov. 1, 2024

Related U.S. Application Data
(60) Provisional application No. 63/337,487, filed on May 2, 2022.
(60) Provisional application No. 63/440,245, filed on Jan. 20, 2023.

(51) Int. Cl. A61K 39/215; A61K 39/00; C07K 14/005; C12N 7/00; C12N 15/82

(52) U.S. Cl. CPC A61K 39/215; C07K 14/005; C12N 7/00; C12N 15/82; A61K 2039/5258; A61K 2039/70; C12N 2770/20022; C12N 2770/20023; C12N 2770/20034; C12N 2770/20051

(57) ABSTRACT

The present disclosure relates to modified coronavirus S protein and virus-like particles (VLPs) comprising modified coronavirus S protein. The present invention also relates to methods of increasing the purity, and/or stability of coronavirus S protein or VLPs comprising modified coronavirus S protein in a host or host cell.

### FIELD OF INVENTION

The present disclosure relates to modified coronavirus S protein and virus-like particles (VLPs) comprising modified coronavirus S protein. The present disclosure also relates to methods of increasing purity, homogeneity and/or stability of coronavirus S protein that are produced in a host or host cell.

### BACKGROUND

Coronaviruses (CoVs) are the largest group of viruses belonging to the Nidovirales order, which includes Coronaviridae, Arteriviridae, Mesoniviridae, and Roniviridae families. The Coronavirinae comprise one of two subfamilies in the Coronaviridae family, with the other being the Torovirinae. The Coronavirinae are further subdivided into four genera, the alpha, beta, gamma, and delta coronaviruses. Members of alpha coronavirus and beta coronavirus are found exclusively in mammals. The alphacoronavirus genus includes two human virus species, HCoV-229E and HCoV-NL63. Important animal alphacoronaviruses are transmissible gastroenteritis virus of pigs and feline infectious peritonitis virus.

Betacoronaviruses of clinical importance to humans are Embecovirus OC43 and HKU1 (which can cause the common cold), Sarbecovirus SARS-CoV and SARS-CoV-2, and Merbecovirus MERS-CoV. Sarbecovirus SARS-CoV-2, also known as 2019-nCoV and HCoV-19, first emerged in 2019 and causes coronavirus disease 2019 (COVID-19), a respiratory illness with high mortality and morbidity resulting in major public health impacts. Outbreaks of SARS-CoV-2, such as the COVID-19 pandemic starting in 2020, are a challenge for healthcare systems due to the asymptomatic incubation period and high transmissibility of the virus. Long-term management of SARS-CoV-2 outbreaks will require high rates of vaccination worldwide with effective vaccines.

Since its initial emergence in 2019, SARS-CoV-2 has mutated into numerous further lineages and sub-lineages through natural substitution and insertion-deletion events from ancestral strains. The phylogeny of these SARS-CoV-2 lineages is typically expressed by PANGO nomenclature (Rambaut et al. 2020). Lineages of clinical importance are further noted as variants of interest (VOI) or variants of concern (VOC) based on the risk they pose to global public health and may be assigned names by the World Health Organization (WHO), for example Beta (corresponding to B.1.351), Gamma (corresponding to P.1), Delta (corresponding to B.1.617.2), and Omicron (corresponding to B.1.1.529) lineages. Mutations vary widely among all reported SARS-CoV-2 lineages, with potential implications for efficacy and development of vaccines comprising coronavirus spike(S) protein.

The club-shaped S protein is the most prominent structural feature of coronaviruses and projects emanating from the surface of the virion. The coronavirus S protein is a glycoprotein that is required for the recognition of host receptors for many coronaviruses as well as the fusion of viral and host cell membranes for viral entry into cells (Belouzard et al., Viruses 2012 June; 4 (6): 1011-33). As the primary glycoprotein on the surface of the viral envelope, S proteins of Coronaviridae are a major target of neutralizing antibodies elicited by natural infection, including SARS-CoV-2 infection, and are key antigens used in coronavirus vaccine formulations.

The SARS-CoV-2 S protein, like S protein of other coronaviruses, is initially synthesized as a precursor protein. Individual precursor S protein forms a homotrimer and undergoes glycosylation within the Golgi compartment as well as processing to remove the signal peptide. The S protein requires a two-step, protease-mediated activation to facilitate membrane fusion. The SARS-CoV-2 S protein is distinguished by a polybasic RRAR furin cleavage site at the S1/S2 junction that is presumably processed in the Golgi compartment to yield two separate polypeptides: the S1 and S2 polypeptide (or subunit), which remain non-covalently bound as S1/S2 protomers within the homotrimer in the prefusion conformation (Walls et al. Cell 2020 181(2) p281-292; Li et al. eLife 2019; 8: e51230). Furin cleavage at the S1/S2 junction and further cleavage at the S2′ site, upstream of the fusion peptide, occurs during viral entry at the cell surface or in endosomes and can be mediated by several proteases. Stabilization of the S protein ectodomain in the prefusion conformation tends to increase the recombinant expression yield, possibly by preventing triggering or misfolding that results from a tendency to adopt the more stable post-fusion structure (Hsieh et al. Science 2020, 369 p.1501-1505).

The S1 domain is further comprised of the N-terminal domain (NTD) and the receptor binding domain (RBD). Neutralizing antibodies from individuals infected with SARS-CoV-2 have been shown to target the RBD of the S1 subunit of the S protein (Premkumar, L., 2020 Science Immunology 11 Jun. 2020: Vol. 5, Issue 48). Highly protective antibodies that are specific to the NTD and target a conserved supersite have also been reported (Lok et al. 2021, Cell Host & Microbe 29).

Vaccination provides protection against disease by inducing a subject to mount an immune response to a likely agent prior to infection. Conventionally, this has been accomplished through the use of live attenuated or whole inactivated forms of the infectious agents as immunogens. To avoid the drawbacks of using a whole virus (such as killed or attenuated viruses) for the making of vaccine, viral proteins or subunits, or recombinant versions thereof, have been pursued as vaccines. A major obstacle to employing viral proteins, either native or recombinant, as vaccine agents is ensuring that the conformation of the protein mimics the antigens in their natural environment. Suitable adjuvants and, in the case of peptides, carrier proteins, may be used to boost the immune response. In addition, viral proteins or subunits as vaccines may elicit primarily humoral responses and thus fail to evoke lasting immunity. Subunit vaccines may be ineffective for diseases in which whole inactivated virus can be demonstrated to provide superior protection.

Virus-like particles (VLPs) may be used in immunogenic compositions to express viral proteins in a preferred conformation with improved antigen presentation to the immune system. VLPs closely resemble mature virions, but they do not contain viral genomic material, and they are non-replicative which contributes to make them safe for administration as a vaccine. In addition, VLPs can be engineered to express viral glycoproteins on the surface of the VLP, which is their native physiological configuration. Since VLPs resemble intact virions and are multivalent particulate structures, VLPs may be more effective in inducing neutralizing antibodies to the glycoprotein than soluble envelope protein antigens.

VLPs self-assemble from single or multiple viral structural protein, such as coronavirus S protein, inside appropriate production host (in vivo assembly). Therefore Coronavirus VLPs can be produced by expressing a recombinant coronavirus S protein in a host. However, the yield, homogeneity and overall quality of the recombinant S protein may be impacted by the degradation of the recombinant protein in the expressing host or host cell and/or during subsequent purification of the protein. Traditionally strategies to minimize protein hydrolysis in hosts such for example plants, including organ-specific transgene expression, organelle-specific protein targeting, the grafting of stabilizing protein domains to labile proteins, protein secretion in natural fluids and the co-expression of companion protease inhibitors. While rational mutagenesis approach might be possible for proteins for which precise information on susceptible cleavage sites is available, in most cases, protein degradation occurs too rapidly to identify the initial cleavage points.

Effective scale-up and manufacture of coronavirus VLPs at the quantity required to achieve widespread vaccination of the global population requires the efficient expression of coronavirus S protein at high quality, stability and purity.

### SUMMARY OF THE INVENTION

The yield, homogeneity and overall quality of a recombinant protein may be impacted by the degradation of the recombinant protein in the expressing host or host cell and/or during subsequent purification of the protein.

The present disclosure provides a modified coronavirus Spike protein (S protein) comprising one or more than one amino acid sequence modification when compared to a corresponding parent or unmodified amino acid sequence. The modified S protein has improved characteristics, such as increased integrity, increased stability, increased resistance against degradation or proteolytic cleavage, increased purity and homogeneity when extracted and/or purified from a host or host cell, or a combination thereof when compared to an unmodified S protein.

In one aspect, it is provided a modified coronavirus S protein, the modified coronavirus S protein comprising one or more than one amino acid sequence modification when compared to a corresponding parent amino acid sequence, wherein the one or more than one modification stabilize the modified coronavirus S protein and wherein the one or more than one modification comprises:
i) a substitution of one or more than one amino acid to introduce a N-glycosylation site at a position corresponding to positions 251, 252 or 253 of reference sequence SEQ ID NO: 1, wherein the N-glycosylation site is asparagine (N) in the consensus sequence N-X-(S or T); or
ii) a deletion of at least four consecutive amino acid residues, wherein the deletion includes at least residues corresponding to positions 249 and 250 of reference sequence SEQ ID NO: 1.

The modified coronavirus S protein comprising i) the substitution of one or more than one amino acid to introduce the N-glycosylation site, may further comprises a deletion of one or more than one amino acids.

The one or more than one the deletion in the modified S protein may comprise the following deletions:
i) at least amino acid residues corresponding to positions 247, 248, 249 and 250 of reference sequence SEQ ID NO: 1;
ii) at least amino acid residues corresponding to positions 248, 249, 250 and 251 of reference sequence SEQ ID NO: 1;
iii) at least amino acid residues corresponding to positions 249, 250, 251 and 252 of reference sequence SEQ ID NO: 1;
iv) at least amino acid residues corresponding to positions 246, 247, 248, 249 and 250 of reference sequence SEQ ID NO: 1;
v) at least amino acid residues corresponding to positions 247, 248, 249, 250 and 251 of reference sequence SEQ ID NO: 1;
vi) at least amino acid residues corresponding to positions 248, 249, 250, 251 and 252 of reference sequence SEQ ID NO: 1;
vii) at least amino acid residues corresponding to positions 246, 247, 248, 249, 250 and 251 of reference sequence SEQ ID NO: 1;
viii) at least amino acid residues corresponding to positions 247, 248, 249, 250, 251 and 252 of reference sequence SEQ ID NO: 1; or
ix) at least amino acid residues corresponding to positions 246, 247, 248, 249, 250, 251 and 252 of reference sequence SEQ ID NO: 1.

The modified coronavirus S protein may comprising i) the substitution of one or more than one amino acid to introduce the N-glycosylation site and may further comprises a deletion of one or more than one amino acids, wherein
i) the N-glycosylation site is introduced at the amino acid corresponding to position 251 of reference sequence SEQ ID NO: 1, and the deletion comprises at least amino acid residues corresponding to positions 249 and 250 of reference sequence SEQ ID NO: 1;
ii) the N-glycosylation site is introduced at the amino acid corresponding to position 252 of reference sequence SEQ ID NO: 1, and the deletion comprises at least amino acid residues corresponding to positions 249 and 250 of reference sequence SEQ ID NO: 1;
iii) the N-glycosylation site is introduced at the amino acid corresponding to position 253 of reference sequence SEQ ID NO: 1, and the deletion comprises at least amino acid residues corresponding to positions 249 and 250 of reference sequence SEQ ID NO: 1;
iv) the N-glycosylation site is introduced at the amino acid corresponding to position 253 of reference sequence SEQ ID NO: 1, and the deletion comprises at least four consecutive amino acid residues, wherein the deletion includes at least residues corresponding to positions 249 and 250 of reference sequence SEQ ID NO: 1;
v) the N-glycosylation site is introduced at the amino acid corresponding to position 253 of reference sequence SEQ ID NO: 1, and the deletion comprises at least amino acid residues corresponding to positions 246, 247, 248, 249, 250, 251 and 252 of reference sequence SEQ ID NO: 1.

The modified coronavirus S protein may comprise a substitution to an asparagine (N) at position corresponding to position 252 or 253 of reference sequence SEQ ID NO: 1 or the amino acid sequence modification comprises a substitution to an asparagine (N) at the position corresponding to position 251 and a substitution to a threonine (T) at position corresponding to position 253 of reference sequence SEQ ID NO: 1.

In a further aspect, the modified S protein may comprise from 80% to 100% identity with the sequence of SEQ ID NO: 8, 10, 12, 16, 20, 39, 41, 43, 45, 47, 49, 51, 53, 55, or 57.

The modified coronavirus S protein may be a chimeric S protein, wherein the chimeric S protein comprises a cytoplasmic tail derived from an influenza hemagglutinin. In one aspect the coronavirus S protein is derived from Betacoronavirus. For example the coronavirus S protein may be derived from lineages A, B, C, or D of Betacoronavirus. In one aspect the coronavirus S protein may be derived from lineage B of Betacoronavirus. Furthermore, the modified S protein may comprise plant specific N-glycans.

It is further provided a genetic construct or nucleic acid comprising a nucleotide sequence encoding the modified coronavirus S protein as described above.

In another aspect it is provided a virus like particle (VLP) or virus like particles (VLPs) comprising the modified S protein as described above. The VLP comprises a greater amount of full-length S protein compared to a VLP that has been assembled from S protein that has not been modified as described herewith. The VLP may further comprise plant lipids.

In a further aspect it is provided a composition comprising a pharmaceutically acceptable carrier, vehicle or excipient and an effective dose of the modified S protein as described herewith or a VLP comprising the modified S protein as described herewith.

In a further aspect a vaccine for inducing an immune response is provided. The vaccine may comprise an effective dose of the modified S protein as described herewith, a VLP comprising the modified S protein as described herewith, or a composition of as described herewith. The vaccine may further comprise an adjuvant, such as AS03.

In yet another aspect, the vaccine may be a multivalent vaccine, comprising a mixture of VLP.

In a further aspect it is provided a method for inducing immunity to a Coronavirus infection in a subject, the method comprising administering the composition or the vaccine as described above to the subject.

In a yet another aspect it is provided a method for inducing an immune response in a subject, the method comprising administering the composition or the vaccine as described above to the subject. An antibody or antibody fragment prepared using the composition or vaccine as described are also provided.

In a further aspect it is provided use of the composition or the vaccine as described above for inducing immunity to a Coronavirus infection in a subject. It is also provided use of the composition or the vaccine as described above for inducing an immune response in a subject.

It is also provided in another aspect a host or host cell comprising the modified S protein, the constructs, nucleic acid and/or VLP as described herewith. The host or host cell may comprise a plant, a portion of a plant, a plant cell, a fungi, a fungi cell, an insect, an insect cell, an animal or an animal cell.

In a further aspect it is provide a method of producing a modified S protein in a host or host cell comprising: a) introducing a nucleic acid comprising a nucleotide sequence encoding the modified coronavirus S protein as described above into the host or host cell, or providing the host or host cell comprising the nucleic acid comprising a nucleotide sequence encoding the modified coronavirus S protein as described above, and b) incubating the host or host cell under conditions that permit the expression of the nucleic acid, thereby producing the modified S protein.

It is also provided a method of producing a modified S protein in a host or host cell comprising: a) expressing the modified S protein as described herewith in the host or host cell, by incubating the host or host cell under conditions that permit the expression of the modified S protein, thereby producing the modified S protein. The modified S protein may further be extracted and purified from the host or host cell.

In a further aspect it is provided a method of producing a virus like particle (VLP) in a host or host cell comprising: a) introducing a nucleic acid comprising a nucleotide sequence encoding the modified coronavirus S protein as described above into the host or host cell, or providing the host or host cell comprising the nucleic acid comprising the nucleotide sequence encoding the modified coronavirus S protein as described above, and b) incubating the host or host cell under conditions that permit the expression of the nucleic acid, thereby producing the VLP. The method may further comprise step c) of harvesting the host or host cell. The VLP may further be extracted and purified from the host or host cell. Also provided is a VLP produced by the method.

In yet another aspect it is provided a method of increasing production of a full-length coronavirus S protein in a host or host cell, the method comprising: a) introducing a nucleic acid comprising a nucleotide sequence encoding the modified coronavirus S protein as described above into the host or host cell, or providing the host or host cell comprising the nucleic acid comprising the nucleotide sequence encoding the modified coronavirus S protein as described above, and b) incubating the host or host cell under conditions that permit the expression of the nucleic acid, thereby producing the modified S protein, wherein a higher amount or higher proportion of the modified S protein is full length modified S protein compared to unmodified S protein produced under similar conditions in the host or host cell.

In a further aspect it is provided a method of producing in a host or host cell a modified coronavirus S protein with increased stability against proteolysis, the method comprising: a) introducing a nucleic acid comprising a nucleotide sequence encoding the modified coronavirus S protein as described above into the host or host cell, or providing the host or host cell comprising the nucleic acid comprising a nucleotide sequence encoding the modified coronavirus S protein as described above, b) incubating the host or host cell under conditions that permit the expression of the nucleic acid, thereby producing the modified S protein with increased stability against proteolysis compared to the stability against proteolysis of an unmodified S protein produced under similar conditions in the host or host cell and; c) optionally extracting the modified S protein from the host or host cell. The modified S-protein may further be purified from the host or host cell. It is also provided a modified S protein produced by the method described above. The modified S protein may exhibit increased stability against proteolysis compared to an S protein that has not been modified as described herewith. VLP or VLPs comprising the modified S-protein are also provided.

In addition it is provided in a further aspect a method of producing a virus-like particle (VLP) with increased full-length S protein content in a host or host cell, the method comprising: a) introducing a nucleic acid comprising a sequence encoding a modified S protein as described herewith into the host or host cell, or providing the host or host cell comprising a nucleic acid comprising a sequence encoding a modified S protein as described herewith b) incubating the host or host cell under conditions that permit the expression of the nucleic acid, thereby producing the VLP, wherein the VLP has an increased full-length S protein content compared to a VLP comprising unmodified S protein that was produced under similar conditions in the host or host cell; and c) optionally extracting the VLP from the host or host cell. Furthermore the VLP may be purified from the host or host cell. Virus-like particle produced by the method are also provided.

In another aspect it is provided a method of increasing stability against proteolysis of a coronavirus S protein produced in a host or host cell, the method comprising: a) modifying a parent coronavirus S protein sequence to produce a modified coronavirus S protein with a modified sequence, wherein the modified coronavirus S protein comprises one or more than one amino acid sequence modification when compared to the parent coronavirus S protein, the one or more than one modification comprising:
i) a substitution of one or more than one amino acid to introduce a N-glycosylation site at a position corresponding to positions 251, 252 or 253 of reference sequence SEQ ID NO: 1, wherein the N-glycosylation site is asparagine (N) in the consensus sequence N-X-(S or T); or
ii) a deletion of at least four consecutive amino acid residues, wherein the deletion includes at least residues corresponding to positions 249 and 250 of reference sequence SEQ ID NO: 1;
   b) expressing the modified coronavirus S protein in the host or host cell, thereby producing a modified S protein with increased stability against proteolysis compared to the stability against proteolysis of the parent coronavirus S protein produced under similar conditions in the host or host cell.

It is further provided a method of modifying a coronavirus S protein to produce a modified coronavirus S protein with one or more than one amino acid sequence modification, wherein the one or more than one amino acid modification stabilize the modified coronavirus S protein, the method comprising:
i) introducing into the coronavirus S protein a substitution of one or more than one amino acid to introduce a N-glycosylation site at a position corresponding to positions 251, 252 or 253 of reference sequence SEQ ID NO: 1, wherein the N-glycosylation site is asparagine (N) in the consensus sequence N-X-(S or T); or
ii) introducing into the coronavirus S protein a deletion of at least four consecutive amino acid residues, wherein the deletion includes at least residues corresponding to positions 249 and 250 of reference sequence SEQ ID NO: 1, thereby modifying the coronavirus S protein.

Modified coronavirus S protein and VLP comprising the modified coronavirus S protein produced by the described methods are also provided.

This summary of the invention does not necessarily describe all features of the invention.

### BRIEF DESCRIPTION OF THE DRAWINGS

FIG. 1A shows a schematic representation of acceptor vector 8716 used for assembly of the vector plasmids encoding modified coronavirus S protein. FIG. 1B shows a schematic representation of vector 9125 encoding modified S protein from the SARS-CoV-2 B lineage with GSAS+2P mutations. FIG. 1C shows a schematic representation of vector 9801 encoding modified S protein from the SARS-CoV-2 B lineage with GSAS+2P+del246-252 mutations. FIG. 1D shows a schematic representation of vector 9802 encoding modified S protein from the SARS-CoV-2 B lineage with GSAS+2P+D253N mutations. FIG. 1E shows a schematic representation of vector 9808 encoding modified S protein from the SARS-CoV-2 B lineage with GSAS+2P+del246-252+D253N mutations. FIG. 1F shows a schematic representation of vector 9513 encoding modified S protein from the SARS-CoV-2 B.1.617.2 lineage with GSAS+2P mutations. FIG. 1G shows a schematic representation of vector 10090 encoding modified S protein from the SARS-CoV-2 B.1.1.529 lineage with GSAS+2P mutations. FIG. 1H shows a schematic representation of vector 10346 encoding modified S protein from SARS-CoV-2 B lineage with GSAS+2P+G252N mutations. FIG. 1I shows a schematic representation of vector 10351 encoding modified S protein from SARS-CoV-2 B lineage with GSAS+2P+P251N+D253T mutations. FIG. 1J shows a schematic representation of vector 10502 encoding modified S protein from SARS-CoV-2 B lineage with GSAS+2P+del247-250 mutations. FIG. 1K shows a schematic representation of vector 10503 encoding modified S protein from SARS-CoV-2 B lineage with GSAS+2P+del248-251 mutations. FIG. 1L shows a schematic representation of vector 10504 encoding modified S protein from SARS-CoV-2 B lineage with GSAS+2P+del249-252 mutations. FIG. 1M shows a schematic representation of vector 10505 encoding modified S protein from SARS-CoV-2 B lineage with GSAS+2P+del246-250 mutations. FIG. 1N shows a schematic representation of vector 10506 encoding modified S protein from SARS-CoV-2 B lineage with GSAS+2P+del247-251 mutations. FIG. 1O shows a schematic representation of vector 10507 encoding modified S protein from SARS-CoV-2 B lineage with GSAS+2P+del248-252 mutations. FIG. 1P shows a schematic representation of vector 10508 encoding modified S protein from SARS-CoV-2 B lineage with GSAS+2P+del246-251 mutations. FIG. 1Q shows a schematic representation of vector 10509 encoding modified S protein from SARS-CoV-2 B lineage with GSAS+2P+del247-252 mutations. FIG. 1R shows a schematic representation of vector 10011 encoding modified S protein from the SARS-CoV-2 B.1.617.2 lineage with GSAS+2P+D253N mutations. FIG. 1S shows a schematic representation of vector 10092 encoding modified S protein from the SARS-CoV-2 B.1.1.529 lineage with GSAS+2P+D253N mutations.

FIG. 2A shows an electron micrograph of virus like particles (VLP) comprising a modified S protein from the SARS-CoV-2 B lineage with GSAS+2P mutations (construct 9125). FIG. 2B shows an electron micrograph of virus like particles (VLP) comprising a modified S protein from the SARS-CoV-2 B lineage with GSAS+2P+del246-252 mutations (GSAS+2P+del246-252; construct 9801). FIG. 2C shows an electron micrograph of virus like particles (VLP) comprising a modified S protein from the SARS-CoV-2 B lineage with GSAS+2P+D253N mutations (GSAS+2P+D253N; construct 9802). FIG. 2D shows an electron micrograph of virus like particles (VLP) comprising a modified S protein from the SARS-CoV-2 B lineage with GSAS+2P+del246-252+D253N mutations (GSAS+2P+del246-252+D253N; construct 9808). FIG. 2E shows an electron micrograph of virus like particles (VLP) comprising a modified S protein from the SARS-CoV-2 C.37 lineage with GSAS+2P mutations (construct 9588).

FIG. 3A shows in planta complete S protein percentage, expressing the following S proteins: parent SARS-CoV-2 B lineage S protein with GSAS+2P mutations (Wt; construct 9125), modified S protein with GSAS+2P+del246-252 mutations (GSAS+2P+del246-252; construct 9801), modified S protein with GSAS+2P+D253N mutations (GSAS+2P+D253N; construct 9802), modified S protein with GSAS+2P+del246-252+D253N mutations (GSAS+2P+del246-252+D253N; construct 9808), and S protein from the SARS-CoV-2 C.37 lineage with GSAS+2P mutations (construct 9588). FIG. 3B shows in planta complete S protein percentage, expressing the following S proteins: parent SARS-CoV-2 B lineage S protein with GSAS+2P mutations (construct 9125), modified SARS-CoV-2 B lineage S protein with GSAS+2P+D253N mutations (construct 9802), parent SARS-CoV-2 B.1.617.2 lineage S protein with GSAS+2P mutations (construct 9513), modified SARS-CoV-2 B.1.617.2 lineage S protein with GSAS+2P+D253N mutations (construct 10011), parent SARS-CoV-2 B.1.1.529 lineage S protein with GSAS+2P mutations (construct 10090), and modified SARS-CoV-2 B.1.1.529 lineage S protein with GSAS+2P+D253N mutations (construct 10092).

FIG. 4A shows purity of a drug substance (DS) obtained from either a host expressing parent SARS-CoV-2 B lineage S protein with GSAS+2P mutations (construct 9125) or modified SARS-CoV-2 B lineage S protein with GSAS+2P+D253N mutations (construct 9802). FIG. 4B shows purity of a drug substance (DS) obtained from hosts expressing the following constructs: parent S protein from the SARS-CoV-2 B lineage with GSAS+2P mutations (Wt; construct 9125), modified S protein with GSAS+2P+del246-252 mutations (GSAS+2P+del246-252; construct 9801), modified S protein with GSAS+2P+D253N mutations (GSAS+2P+D253N; construct 9802), modified S protein with GSAS+2P+del246-252+D253N mutations (GSAS+2P+del246-252+D253N; construct 9808), and S protein from the SARS-CoV-2 C.37 lineage with GSAS+2P mutations (construct 9588).

FIG. 5 shows increased stability of purified modified S protein (SARS-CoV-2 B lineage with GSAS+2P+D253N mutations: construct 9802) compared to an unmodified (parent) S protein (SARS-CoV-2 B lineage with GSAS+2P mutations: construct 9125) following overnight incubation at 24° C. The modified S protein and unmodified S protein were either extracted using enzymatic extraction methods at pH5.5 (Enz. pH5.5) or pH 6.1 (Enz. pH6.1) or by mechanical extraction method. (Mech).

FIG. 6A shows a Western blot analysis of the following S proteins: parent SARS-CoV-2 B lineage S protein with GSAS+2P mutations (construct 9125), modified SARS-CoV-2 B lineage S protein with GSAS+2P+D253N mutations (construct 9802), modified SARS-CoV-2 B lineage S protein with GSAS+2P+G252N mutations (construct 10346), and modified SARS-CoV-2 B lineage S protein with GSAS+2P+P251N+D253T mutations (construct 10351). FIG. 6B shows in planta complete S protein percentage, expressing the following S proteins: parent SARS-CoV-2 B lineage S protein with GSAS+2P mutations (construct 9125), modified SARS-CoV-2 B lineage S protein with GSAS+2P+D253N mutations (construct 9802), modified SARS-CoV-2 B lineage S protein with GSAS+2P+G252N mutations (construct 10346), and modified SARS-CoV-2 B lineage S protein with GSAS+2P+P251N+D253T mutations (construct 10351). FIG. 6C shows an electron micrograph of virus like particles (VLP) comprising modified SARS-CoV-2 B lineage S protein with GSAS+2P+D253N mutations (construct 9802), modified SARS-CoV-2 B lineage S protein with GSAS+2P+G252N mutations (construct 10346), and modified SARS-CoV-2 B lineage S protein with GSAS+2P+P251N+D253T mutations (construct 10351).

FIG. 7 shows in planta complete S protein percentage, expressing the following S proteins: parent SARS-CoV-2 B lineage S protein with GSAS+2P mutations (construct 9125), modified SARS-CoV-2 B lineage S protein with GSAS+2P+del246-252; (construct 9801); modified SARS-CoV-2 B lineage S protein with GSAS+2P+del247-250; (construct 10502); modified SARS-CoV-2 B lineage S protein with GSAS+2P+del248-251; (construct 10503); modified SARS-CoV-2 B lineage S protein with GSAS+2P+del249-252; (construct 10504); modified SARS-CoV-2 B lineage S protein with GSAS+2P+del246-250; (construct 10505); modified SARS-CoV-2 B lineage S protein with GSAS+2P+del247-251; (construct 10506); modified SARS-CoV-2 B lineage S protein with GSAS+2P+del248-252; (construct 10507); modified SARS-CoV-2 B lineage S protein with GSAS+2P+del246-251; (construct 10508); modified SARS-CoV-2 B lineage S protein with GSAS+2P+del247-252; (construct 10509).

### DETAILED DESCRIPTION

The following description is of a preferred embodiment.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments, consist essentially of those elements and/or steps, and in other embodiments consist of those elements and/or steps, whether or not these embodiments are specifically referred to. In addition, the use of the singular includes the plural, and “or” means “and/or” unless otherwise stated. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−10% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

Modified coronavirus Spike protein (S protein), also referred to as “modified coronavirus S protein” or “modified S protein” and methods of producing modified S protein in host or host cell are described herein. The modified S protein may comprise one or more than one modification compared to a parent (unmodified) or wild type S protein. It has been observed that modifications, for example substitution or deletion of specific amino acids in coronavirus S proteins, for example S protein from the B lineage of SARS-CoV-2, result in improved characteristics of the modified S protein when compared to the parent (unmodified) or wild type (unmodified) S protein.

By “modification”, “amino acid modification”, or “amino acid sequence modification” it is meant a mutation, substitution, replacement or deletion of one or more than one amino acid residues in a sequence compared to the original parent (unmodified) sequence. The parent sequence may be a wild type sequence or the parent sequence may be a sequence that already comprises modifications (“parent modifications”) when compared to a wild type sequence. By “amino acid substitution” or “substitution” it is meant the replacement of an amino acid in the amino acid sequence of a protein with a different amino acid. The terms amino acid, amino acid residue or residue are used interchangeably in the disclosure. One or more than one amino acids may be replaced with one or more amino acids that are different than the original amino acid at this position, without changing the overall length of the amino acid sequence of the protein. The substitution or replacement may be experimentally induced by altering the codon sequence in a nucleotide sequence encoding the protein to the codon sequence of a different amino acid compared to the original amino acid. Furthermore one or more than one amino acids may be deleted from the amino acid sequence of the protein. The resulting protein is a modified S protein. The modified S protein does not occur naturally.

The modified S protein includes non-naturally occurring S protein, having at least one modification compared to a parent S protein and having improved characteristics compared to parent S protein from which the amino acid sequence of the modified S protein is derived. Modified S proteins have an amino acid sequence, not found in nature, which is derived by replacement of one or more amino acid residues of an S protein with one or more different amino acids.

The parent S protein may also be referred to as unmodified S protein. If the parent is refer to as “unmodified”, it is meant that the parent sequence does not comprise the substitutions and/or deletions as they are described herewith. However, the parent S protein may comprise other modifications compared to a wild type sequence. In some embodiments, the parent or unmodified S protein may be a wild type HA. In other embodiments, the parent or unmodified S protein may comprise other modifications as described below. For example the parent S protein may comprise one or more than one substitution or replacement to stabilize the coronavirus S protein or coronavirus S protein trimer in a prefusion conformation. Furthermore, the parent S protein may be a chimeric S protein. For example, the ectodomain and the transmembrane domain (TM) or portion of the TM of the parent S protein may be derived from a Coronavirus S protein (such as SARS-CoV 2), and the cytoplasmic tail (CT) or portion of the CT may be derived from influenza HA.

By “parent S protein” it is meant the S protein from which the modified S protein may be derived. For example the parent S protein may be modified to produce a modified S protein having the modification as described herewith. As further described below, the parent S protein may be from a coronavirus of a first variant or lineage (also referred as “acceptor” variant or lineage), for example the coronavirus B lineage and the one or more modifications may be derived or determined from an S protein from a coronavirus from a second variant or lineage (also referred to as “donor” variant or lineage), for example the coronavirus C lineage.

Some of the residues identified for modification, mutation or substitution correspond to conserved residues whereas others are not. In the case of residues which are not conserved, the replacement of one or more amino acids is limited to substitutions which produce a modified S protein which has an amino acid sequence that does not correspond to one found in nature. In the case of conserved residues, such modification, substitution or replacements should also not result in a naturally occurring S protein sequences.

### Conserved Substitutions

As described herein, residues in S proteins may be identified and modified, substituted or mutated to produce modified S protein. The substitutions or mutations at specific positions are not limited to the amino acid substitutions described herewith or as given in the examples. For example, the S protein may contain conserved or conservative substitutions of describes amino acid substitutions.

As used herein, the term “conserved substitution” or “conservative substitution” and grammatical variations thereof, refers to the presence of an amino acid residue in the sequence of the S protein that is different from, but is in the same class of amino acid as the described substitution or described residue (i.e., a nonpolar residue replacing a nonpolar residue, an aromatic residue replacing an aromatic residue, a polar-uncharged residue replacing a polar-uncharged residue, a charged residue replacing a charged residue). In addition, conservative substitutions can encompass a residue having an interfacial hydropathy value of the same sign and generally of similar magnitude as the residue that is replacing the wildtype residue.

Conservative amino acid substitutions are likely to have a similar effect on the activity of the resultant modified S protein, as the original substitution or modification. Further information about conservative substitutions can be found, for instance, in Ben Bassat et al. (J. Bacteriol, 169:751-757, 1987), O'Regan et al. (Gene, 77:237-251, 1989), Sahin-Toth et al. (Protein ScL, 3:240-247, 1994), Hochuli et al (Bio/Technology, 6:1321-1325, 1988) and in widely used textbooks of genetics and molecular biology.

The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected.

Accordingly, the present description relates to a modified coronavirus Spike protein (S protein) comprising one or more than one amino acid sequence modification when compared to a corresponding parent or unmodified amino acid sequence. It has been found that naturally occurring sequence mutations or modifications that are specific to the S protein of one coronavirus variant or lineage may confer desirable or improved characteristics to a S protein that does not naturally have these mutations or modifications, such for example an S protein from a different lineage or variant. It has further been found, that non-naturally occurring sequence mutations or modifications in the N-terminal region of the S protein may also confer desirable or improved characteristics to the S protein. The modified S protein therefore may comprise mutations or modifications from an S protein from a different lineage or variant, and/or the modified S protein may comprise modification that are not naturally occurring i.e. that are not found in a different lineage or variant and the S protein may exhibit improved characteristics as compared to the wild-type or unmodified S protein.

In one aspect, the modified S protein comprises one or more than one amino acid sequence modification when compared to a corresponding parent amino acid sequence, wherein the one or more than one modification correspond to amino acids at positions 246, 247, 248, 249, 250, 251, 252, 253, or a combination thereof, of reference sequence SEQ ID NO: 1.

In one aspect, the modification as described herewith may comprise one or more than one deletion in the N-terminal region of the protein. For example, the modified S protein may comprise one or more than one deletion correspond to amino acids at positions 246, 247, 248, 249, 250, 251, 252, or a combination thereof, of reference sequence SEQ ID NO: 1.

In another aspect, the modification as described herewith may introduce one or more than one N-glycosylation site into the modified S protein. Accordingly, the modifies S protein may comprise one or more than one N-Glycosylation site, when compared to the parent (unmodified) S protein, wherein the N-glycosylation site is asparagine (N) in the consensus sequence N-X-T/S (wherein X is any amino acid except proline). The N-Glycosylation site may be introduced at positions that correspond to positions 251, 252 or 253 of reference sequence SEQ ID NO: 1.

Examples of improved characteristics of the modified S protein include but are not limited to: increased integrity, increased stability, increased resistance to degradation or proteolytic cleavage or proteolysis, increased purity and homogeneity when extracted and/or purified from a host or host cell, or a combination thereof of the recombinant modified S protein when expressed in a host or host cell as compared to the unmodified S protein; improved integrity, stability, or both integrity and stability of the modified S protein when expressed in a host or host cell as compared to the unmodified S protein; increased or improved resistance against degradation, proteolysis, cleavage or hydrolysis (also referred to as “clipping”) of the modified S protein when expressed in a host or host cell as compared to the unmodified S protein; decrease of heterogeneity and/or truncation of the modified S protein when expressed in a host or host cell as compared to the unmodified S protein; improved processing and/or folding of the modified S protein when expressed in a host or host cell as compared to the unmodified S protein.

Coronavirus S protein (for example parent S protein, wild type S protein or unmodified S protein) that may be modified as described herein to improve characteristics of the S protein, for example having increased stability, integrity, purity, homogeneity or a combination thereof including new coronavirus S proteins that emerge over time due to natural modifications of the S protein amino acid sequence (for example new S protein variants as described below), or non-native S proteins, that may be produced as a result of altering the S proteins (e.g. chimeric S proteins, or S proteins that have been altered to achieve a desirable property, for example, increasing expression within a host or stabilization of the S protein in a prefusion conformation). Similarly, modified S proteins as described herein, may be derived from wild type S proteins, novel S proteins that emerge over time due to natural modifications of the S amino acid sequence, non-modified S proteins, non-native S proteins for example, chimeric S proteins, or S proteins that have been altered to achieve a desirable property, for example, increasing expression of S protein or increased production of VLP comprising S protein within a host.

The modified S protein of the current disclosure may comprise one or more modifications that have been derived from an S protein from a coronavirus from a different variant or lineage compared to the S protein that has been modified. The modified S protein may be derived from a coronavirus of a first variant or lineage (also referred as “acceptor” variant or lineage), for example the coronavirus B lineage and the one or more modifications may be derived or determined from an S protein from a coronavirus from a second variant or lineage (also referred to as “donor” variant or lineage), for example the coronavirus C lineage.

Coronavirus variant (for example SARS-CoV-2 variants) refers to mutant specimens of the coronavirus (also referred to a genetic variant of the coronavirus) that contains one or more mutations when compared to other virus variants such for example the original or ancestral virus of SARS-CoV-2. Generally, a coronavirus variant has one or more mutations that differentiate it from other variants of the virus. A coronavirus genetic variant is genetically distinct from other variants, but not sufficiently different to be termed a distinct virus strain.

While there are many thousands of variants of coronavirus, for example SARS-CoV-2, subtypes of the virus can be put into larger groupings such as lineages or, subgenera or clades.

A lineage, subgenera or clade are a genetically closely related group of virus variants derived from a common ancestor. The coronavirus lineages, subgenera or clades may use different nomenclatures, such for example the clades identified by the Global Initiative on Sharing Avian Influenza Data (GISAID) or by “Nextstrain” (Hadfield et al. Nextstrain: real-time tracking of pathogen evolution, Bioinformatics (2018)) or lineages as defined by PANGO nomenclature (see Rambaut et al. 2020, which is incorporated by reference), or system of lineage nomenclature as known in the art. In addition, the World Health Organization (WHO) has adopted a nomenclature restricted to variants of interest (VOI) and variants of concern (VOC) where the variants are named with letters of the Greek alphabet to ease discussion and communication with non-scientific audience. For example Alpha refers to B.1.1.7 (Pango lineage), Beta refers to B1.1.351 (Pango lineage), Gamma refers to P.1 (Pango lineage), Delta refers to B.1.617.2 (Pango lineage), Lambda refers to C.37 (Pango lineage) or Omicron refers to B.1.1.529 (Pango lineage) (see https://www.who.int/en/activities/tracking-SARS-CoV-2-variants/).

The donor or acceptor lineage may be a SARS-CoV-2 lineage. The donor or acceptor lineage may be defined by PANGO nomenclature (see Rambaut et al. 2020, which is incorporated by reference) or another system of lineage nomenclature.

For example, the donor coronavirus lineage may be selected from the A, B, C, Q, L, D, P, N, S, AE, AF, AZ, AY, BA, W, Y, or Z lineages, as defined by PANGO nomenclature. For example, the donor coronavirus lineage may be the C lineage. For example, the donor coronavirus lineage may be the C.37 (“Lambda”) lineage.

For example, the acceptor coronavirus lineage may be selected from the A, B, C, Q, L, D, P, N, S, AE, AF, AZ, AY, BA, W, Y, or Z lineages, as defined by PANGO nomenclature. For example, the acceptor coronavirus lineage may be the B lineage. For example, the acceptor coronavirus lineage may be selected from the B lineage, the B.1.1.7 (“Alpha”) lineage, the B.1.351 (“Beta”) lineage, the P.1 (“Gamma”) lineage, the B.1.617.2 (“Delta”) lineage, the B.1.1.529 (“Omicron”) lineage, or another SARS-CoV-2 lineage. For example, the acceptor coronavirus lineage may be of any coronavirus lineage, as long as the lineage is different from the donor lineage. For example, the acceptor coronavirus lineage may be of any lineage, with the exception of the C lineage. In one embodiment the modifications may be derived from an S protein from the C lineage (donor) and parent S protein that is being modified is derived from the B lineage (acceptor).

In one embodiment, modified S protein comprises modifications from donor lineage C.37 (“Lambda”) S protein introduced to acceptor lineage B coronavirus S protein and expressed in a host or host cell, such as plants or plant cells.

For example, the C.37 (“Lambda”) lineage comprises the following modifications when compared to the S protein of the ancestral B lineage (P0DTC2, SEQ ID NO: 1): G75V, T76I, del246-252, D253N, L452Q, F490S, D614G, and T859N.

It was found that the introduction of modification of amino acid residues that are found in the N-terminal domain of the C.37 lineage S protein into B lineage S proteins (modified S protein) lead to improved characteristics of the B lineage S protein when produced in a host, such as a plant, compared to the unmodified (parent) B lineage S protein. It was further found that characteristics of the S protein could also be improved when using a subset of modifications as further described herewith. In another aspect, it was further found that the introduction of one or more than one N-glycosylation site into the modified S protein also improved the characteristics of the S protein.

Accordingly, the modified S protein may comprise one or more than one modification, mutation or substitution that are located at the N-terminal domain of the S protein.

In one aspect the modified S protein may comprise one or more than one modification, mutation or substitution corresponding to positions 246, 247, 248, 249, 250, 251, 252, 253, or a combination thereof.

### Deletion

The modified S protein may comprise one or more than one deletion that correspond to positions 246, 247, 248, 249, 250, 251 or 252 of reference sequence SEQ ID NO: 1. For example, the modified S protein may comprise a deletion of at least four consecutive amino acid residues, wherein at least residues corresponding to positions 249 and 250 of reference sequence SEQ ID NO: 1 are deleted. For example, the modified S protein may comprise at least the deletion of residues corresponding to positions 247, 248, 249 and 250 of reference sequence SEQ ID NO: 1. In another example, modified S protein may comprise at least the deletion of residues corresponding to positions 248, 249, 250 and 251 of reference sequence SEQ ID NO: 1. In a further example, the modified S protein may comprise at least the deletion of residues corresponding to positions 249, 250, 251 and 252 of reference sequence SEQ ID NO: 1. In another example the modified S protein may comprise at least the deletion of residues corresponding to positions 246, 247, 248, 249 and 250 of reference sequence SEQ ID NO: 1. In a further example, the modified S protein may comprise at least the deletion of residues corresponding to positions 247, 248, 249, 250 and 251 of reference sequence SEQ ID NO: 1. In yet another example, the modified S protein may comprise at least the deletion of residues corresponding to positions 248, 249, 250, 251 and 252 of reference sequence SEQ ID NO: 1. In yet another example, the modified S protein may comprise at least the deletion of residues corresponding to positions 246, 247, 248, 249, 250 and 251 of reference sequence SEQ ID NO: 1. In yet another example, the modified S protein may comprise at least the deletion of residues corresponding to positions 247, 248, 249, 250, 251 and 252 of reference sequence SEQ ID NO: 1. In a further example, the modified S protein may comprise at least the deletion of residues corresponding to positions 246, 247, 248, 249, 250, 251 and 252 of reference sequence SEQ ID NO: 1. In a non-limiting example, the modified S protein may comprise the deletions shown in Table 2.

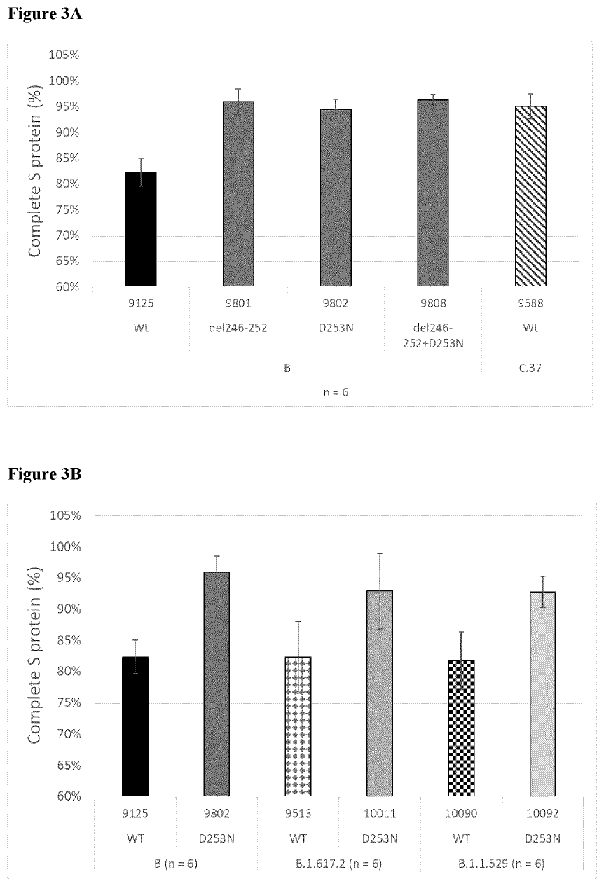

As shown in FIG. 3A, the proportion of full-length coronavirus S protein versus cleaved coronavirus S protein (“Complete S protein (%)”) increased from approximately 80% full-length coronavirus S protein for the parent (B acceptor) S protein (construct 9125) to approximately 95% full-length coronavirus S protein for the modified S protein wherein the residues that correspond to positions 246-252 of reference sequence SEQ ID NO: 1 have been deleted (constructs 9801).

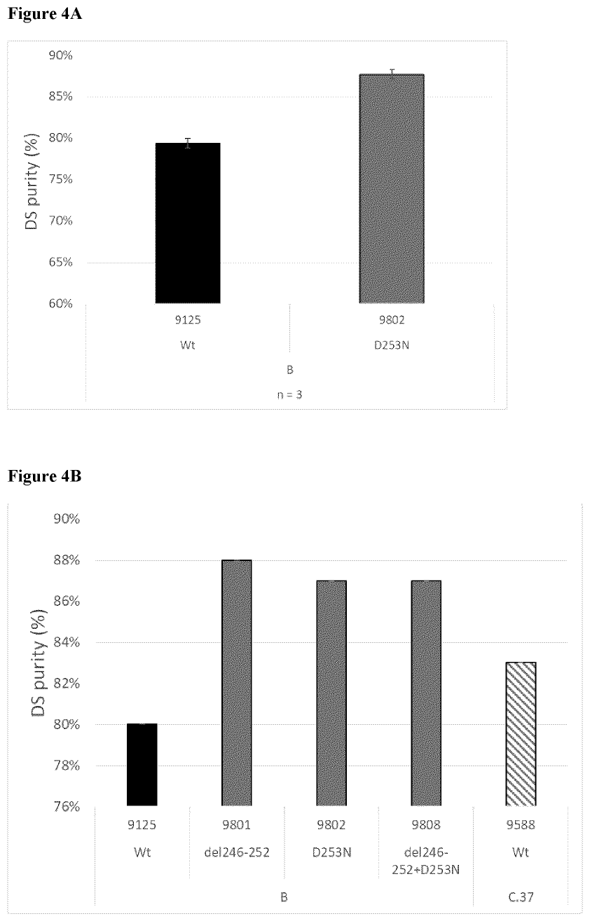

As further shown in FIG. 4B, clarification and purification of the coronavirus S protein followed by quantification on SDS gel shows an increase in drug substance (DS) purity (%). When modified S protein incorporating del246-252 (construct 9801) modifications from donor lineage C.37 (“Lambda”) S protein was extracted and purified from plants, an increase in DS purity was observed compared to the purity of a DS that was obtained from plants that expressed parent (unmodified) S protein (construct 9125). Notably, the increase in DS purity is also greater for DS obtained from modified coronavirus S protein incorporating del246-252 (construct 9801) than the purity of DS obtained from coronavirus S protein from donor lineage C.37 (“Lambda”, construct 9588) expressed in the same system.

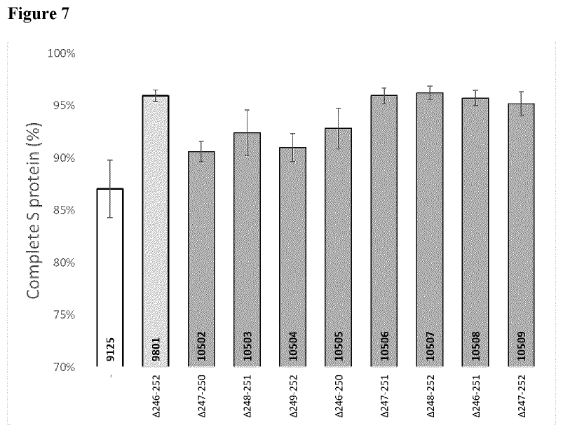

As further shown in FIG. 7, the proportion of full-length coronavirus S protein versus cleaved coronavirus S protein (“Complete S protein (%)”) also increased for modified S protein comprising the following deletions: del 246-252 (construct 9801), del247-250 (construct 10502), del248-251 (construct 10503), del249-252 (construct 10504), del246-250 (construct 10505), del247-251 (construct 10506), del248-252 (construct 10507), del246-251 (construct 10508) and del247-252 (construct 10509), when compared to the parent (unmodified) S protein (construct 9125). More specifically, the proportion of full-length coronavirus S protein versus cleaved coronavirus S protein (“Complete S protein (%)”) increased to approximately 91%-96% for modified S protein comprising the deletions as described above compared to about 87% with the corresponding parent S proteins (constructs 9125).

### Substitution

In one aspect, the modification as described herewith may introduce one or more than one N-glycosylation site into the modified S protein. Accordingly, the modified S protein may comprise one or more than one N-Glycosylation site, when compared to the parent (unmodified) S protein, wherein the N-glycosylation site is asparagine (N) in the consensus sequence of N-X-T/S (wherein X is any amino acid except proline).

The modified S protein may comprise one or more than on substitution corresponding to position 251, 252 or 253 of the reference sequence SEQ ID NO: 1. The one or more than one substitution may introduce a N-glycosylation site into the modified S protein. The N-glycosylation site is asparagine (N) in the consensus sequence N-X-(S or T) wherein X can be any amino acid except proline. For example, the modified S protein may comprise a substitution corresponding to position 252 or position 253 of reference sequence SEQ ID NO: 1, or the modified S protein may comprise substitutions that correspond to positions 251 and 253 of reference sequence SEQ ID NO: 1. The substitution may introduce a N-glycosylation site at a position that corresponds with position 251, 252 or 253 of reference sequence SEQ ID NO: 1.

For example, the amino acid residue corresponding to position 253 of reference sequence SEQ ID NO: 1, may be modified from aspartic acid (D) to asparagine (N) (D253N) to create an N-Glycosylation site at position 253. Furthermore, amino acid residue corresponding to position 252 of reference sequence SEQ ID NO: 1, may be modified from glycine (G) to asparagine (N) (G252N) to create an N-Glycosylation site at position 252. In addition, the residue corresponding to position 251 of reference sequence SEQ ID NO: 1, may be modified from proline (P) to asparagine (N) (P251N) and the residue corresponding to position 253 of reference sequence SEQ ID NO: 1, may be modified from aspartic acid (D) to threonine (T) (D253T) to create an N-Glycosylation site at position 251.

As shown in FIG. 3B, a greater proportion of modified S protein was observed as full length S protein (expressed as “Complete S protein (%)”), when the S protein was derived from the B lineage (construct 9802) B.1.617.2 lineage (construct 10011) or B.1.1.529 lineage (construct 10092) and comprised a substitution that corresponds to position 253 (“modified S protein”), compared to the corresponding unmodified S proteins (constructs 9125, 9513 and 10090, respectively).

More specifically, the proportion of full-length coronavirus S protein versus cleaved coronavirus S protein (“Complete S protein (%)”) increased to approximately 93%-96% for modified S protein comprising a D253N substitution and that are derived from the B lineage (construct 9802), B.1.617.2 lineage (construct 10011) or B.1.1.529 lineage (construct 10092) compared to about 81%-82% with the corresponding unmodified S proteins (constructs 9125, 9513 and 10090, respectively).

Furthermore, as shown in FIGS. 4A and 4B, clarification and purification of the coronavirus S protein followed by quantification on SDS gel shows an increase in drug substance (DS) purity (%). When modified S protein incorporating D253N (construct 9802) modifications from donor lineage C.37 (“Lambda”) S protein was extracted and purified from plants, an increase in DS purity was observed compared to the purity of a DS that was obtained from plants that expressed unmodified S protein (construct 9125). Notably, the increase in DS purity is also greater for DS obtained from modified coronavirus S protein D253N (construct 9802) than the purity of DS obtained from coronavirus S protein from donor lineage C.37 (“Lambda”, construct 9588) expressed in the same system (FIG. 4B).

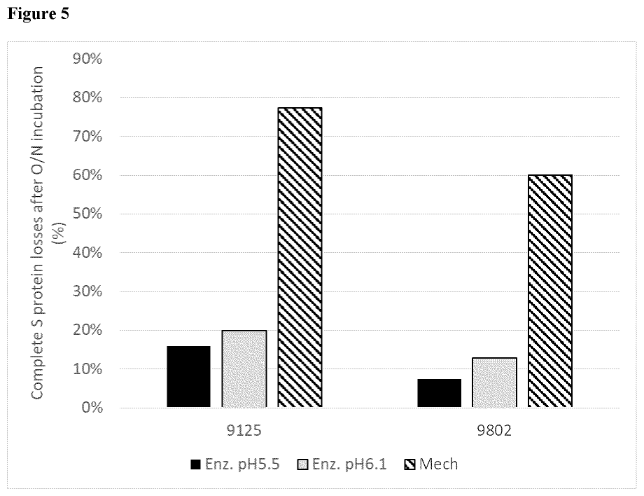

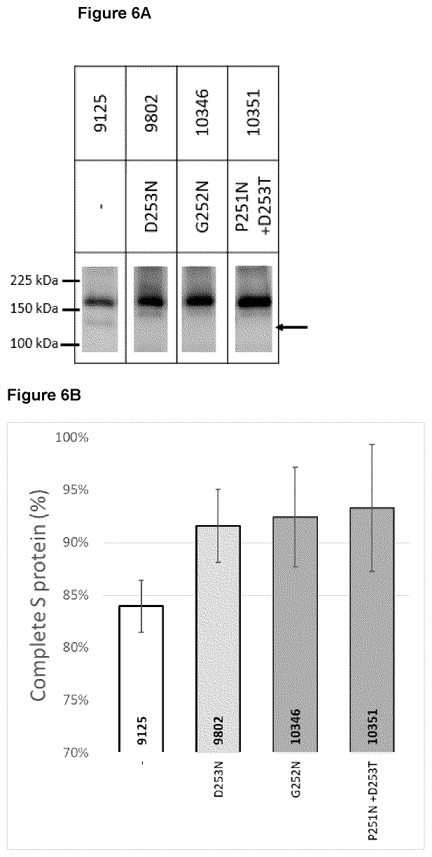

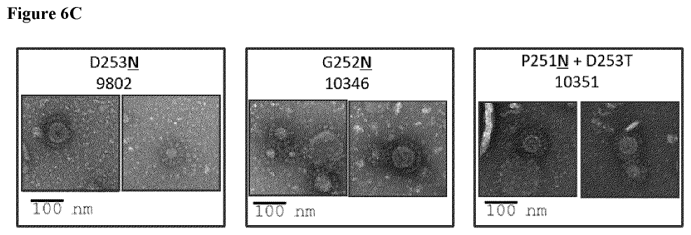

As further shown in FIGS. 6A and 6B, when glycosylation sites were introduced into the modified S protein at the residues that corresponds with position 253, 252 or 251 of reference sequence SEQ ID NO: 1, the proportion of full-length S protein versus cleaved or truncated (“clipped”) S protein (expressed as “Complete S protein (%)” in FIG. 6B) is significantly increased for the S protein with glycosylation sites at position 253, 252 or 251 compared to the parent S protein.

More specifically, as shown in FIG. 6B, a greater proportion of modified S protein was extracted and purified as full length S protein from plants (expressed as “Complete S protein (%)”), when the S protein comprised substitutions that corresponds to position 253 (construct 9802), position 252 (construct 10346) or positions 251 and 253 (construct 10351), when compared to the corresponding unmodified S proteins (constructs 9125).

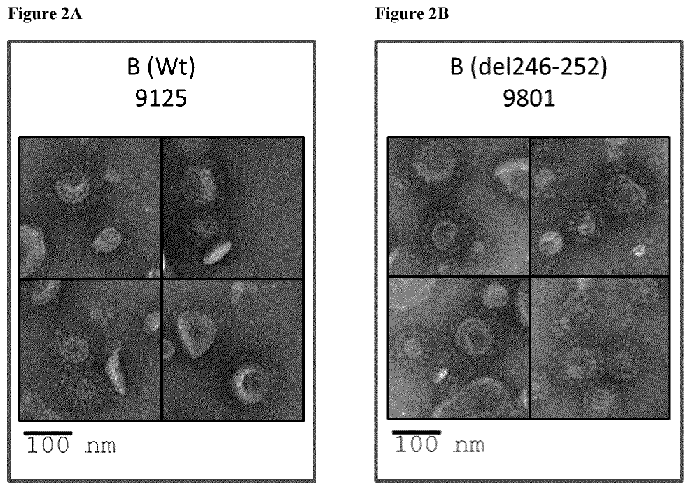

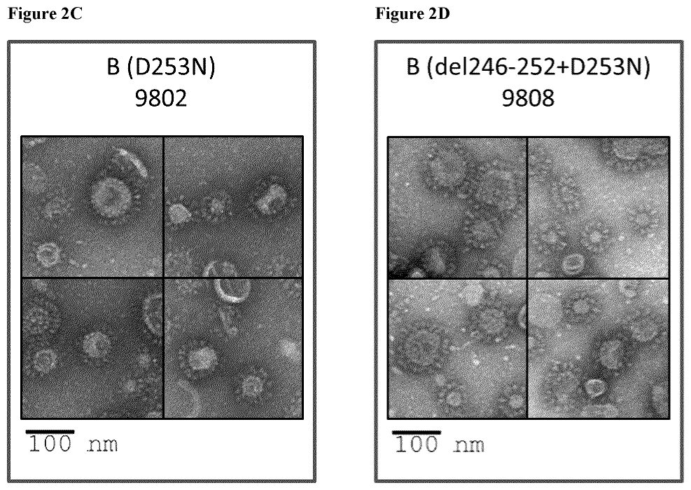

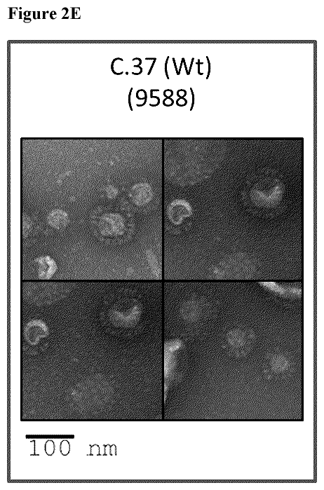

As further shown in FIGS. 6C, when expressed in N. benthamiana, the modified S proteins with additional glycosylation sites corresponding to position 253, 252 or 251 (constructs: 9802, 10346 and 10351) were observed to form VLPs similar to the unmodified SARS-CoV-2 S protein (FIG. 2A, construct 9125).

The modified S protein with a D253N modification (construct 9802) that had been extracted by enzyme extraction methods at pH5.5 (Enz. pH5.5) or pH 6.1 (Enz. pH6.1) or by mechanical extraction method (Mech), exhibited an increased stability compared to an unmodified S protein (construct 9125) following overnight incubation at 24° C. (see FIG. 5).

### Deletion and Substitution

In a further aspect, the modified S protein may comprise one or more than one deletion that correspond to positions 246, 247, 248, 249, 250, 251, or 252, and one or more than one substitution that corresponds to position 251, 252 or 253 of reference sequence SEQ ID NO: 1.

For example, the modified S protein may comprise one or more than one deletion and one or more than one substitution, wherein the one or more than one deletion comprises at least four consecutive amino acid residues and wherein at least residues corresponding to positions 249 and 250 of reference sequence SEQ ID NO: 1 are deleted and, wherein the one or more than one substitution correspond to position 251, 252, or 253 of reference sequence SEQ ID NO: 1.

For example, the modified S protein may comprise at least the deletion of residues corresponding to positions 247, 248, 249 and 250 of reference sequence SEQ ID NO: 1 and one or more than one substitution corresponding to position 251, 252, or 253 of reference sequence SEQ ID NO: 1. In another example, modified S protein may comprise at least the deletion of residues corresponding to positions 248, 249, 250 and 251 of reference sequence SEQ ID NO: 1 and one or more than one substitution corresponding to position 252, or 253 of reference sequence SEQ ID NO: 1. In a further example, the modified S protein may comprise at least the deletion of residues corresponding to positions 249, 250, 251 and 252 of reference sequence SEQ ID NO: 1 and a substitution corresponding to position 253 of reference sequence SEQ ID NO: 1. In another example the modified S protein may comprise at least the deletion of residues corresponding to positions 246, 247, 248, 249 and 250 of reference sequence SEQ ID NO: 1 and one or more than one substitution corresponding to position 251, 252, or 253 of reference sequence SEQ ID NO: 1. In a further example, the modified S protein may comprise at least the deletion of residues corresponding to positions 247, 248, 249, 250 and 251 of reference sequence SEQ ID NO: 1 and one or more than one substitution corresponding to position 252, or 253 of reference sequence SEQ ID NO: 1. In yet another example, the modified S protein may comprise at least the deletion of residues corresponding to positions 248, 249, 250, 251 and 252 of reference sequence SEQ ID NO: 1 and a substitution corresponding to position 253 of reference sequence SEQ ID NO: 1. In yet another example, the modified S protein may comprise at least the deletion of residues corresponding to positions 246, 247, 248, 249, 250 and 251 of reference sequence SEQ ID NO: 1 and one or more than one substitution corresponding to position 252, or 253 of reference sequence SEQ ID NO: 1. In yet another example, the modified S protein may comprise at least the deletion of residues corresponding to positions 247, 248, 249, 250, 251 and 252 of reference sequence SEQ ID NO: 1 and a substitution corresponding to position 253 of reference sequence SEQ ID NO: 1. In a further example, the modified S protein may comprise at least the deletion of residues corresponding to positions 246, 247, 248, 249, 250, 251 and 252 of reference sequence SEQ ID NO: 1 and a substitution corresponding to position 253 of reference sequence SEQ ID NO: 1.

As shown in FIG. 3A, the proportion of full-length coronavirus S protein (“complete S protein (%)”) versus cleaved coronavirus S protein is significantly increased for modified S protein incorporating the deletion del246-252 and the substitution D253N (‘del-246-252+D253N’; construct 9808). More specifically, the proportion of full-length coronavirus S protein versus cleaved coronavirus S protein (“Complete S protein (%)”) increased from approximately 82% full-length coronavirus S protein for the B acceptor strain S protein (construct 9125) to approximately 96% full-length coronavirus S protein with modified S protein incorporating the deletion and substitution (‘del-246-252+D253N’; construct 9808).

As further shown in FIGS. 4A and 4B, clarification and purification of the coronavirus S protein followed by quantification on SDS gel shows an increase in drug substance (DS) purity (%). When modified S protein incorporating del246-252+D253N (construct 9808) modifications from donor lineage C.37 (“Lambda”) S protein was extracted and purified from plants, an increase in DS purity was observed compared to the purity of a DS that was obtained from plants that expressed unmodified S protein (construct 9125). Notably, the increase in DS purity is also greater for DS obtained from modified coronavirus S protein incorporating del246-252+D253N (construct 9808) than the purity of DS obtained from coronavirus S protein from donor lineage C.37 (“Lambda”, construct 9588) expressed in the same system (FIG. 4B).

By “correspond to an amino acid”, “corresponding to an amino acid” or “correspond to position” or “corresponding to position” and the like, it is meant that an amino acid corresponds to an amino acid in a sequence alignment with a coronavirus S protein reference sequence as described below.

Modifications from the S protein donor lineage nucleic sequence may be introduced into the corresponding nucleic acid positions of the S protein acceptor lineage nucleic sequence. Similarly, amino acid substitutions or deletions from the S protein donor lineage amino acid sequence may be introduced into the corresponding amino acid positions of the S protein acceptor lineage sequence. As further described herewith, additional modifications that are not found in the S protein from the donor lineage may also be introduced into the modified S protein.

Throughout this disclosure, the amino acid residue number or residue position of coronavirus S protein is in accordance with the numbering of a S protein reference sequence. For example, the S protein reference sequence is the sequence of the “ancestral” B lineage SARS-CoV-2 S protein (UniProtKB-PODTC2, SEQ ID NO: 1). The corresponding amino acid positions may be determined by aligning the sequences of the S protein with the S protein reference sequence. For example an alignment of amino acid sequences shows that positions 246-253 of SARS-CoV-2 B (SEQ ID NO: 1) correspond to positions 256-263 in the sequence of SARS-CoV-2 B.1.617.2 (SEQ ID NO. 14) and positions 255-262 in the sequence of SARS-CoV-2 B.1.1.529 (SEQ ID NO: 18). Corresponding positions in other sequences may be determined by similar alignments. Methods of alignment of sequences for comparison are well-known in the art and further described below.

The modified coronavirus S protein may comprise one or more than one amino acid sequence modifications when compared to a corresponding unmodified (parent) amino acid sequence, wherein the one or more than one modification corresponding to amino acids at positions 246, 247, 248, 249, 250, 251, 252, 253, or a combination thereof, of reference sequence SEQ ID NO: 1.

The one or more than one amino acid sequence modification may comprises a substitution of one or more than one amino acid or a deletion of one or more than one amino acid. For example, the amino acid sequence modification comprises a substitution to a non-glycine corresponding to position 252, such for example a substitution to an asparagine (N) corresponding to position 252. In one embodiment the modification is a G252N substitution and therefore the modified S protein comprises a G252N substitution or modification.

In another example, the modification comprises a substitution to a non-aspartic acid corresponding to position 253, such for example a substitution to an asparagine (N) corresponding to position 253. In one embodiment the modification is a D253N substitution and therefore the modified S protein comprises a D253N substitution or modification.

The modification may further comprise two substitutions. For example, the modified S protein may comprise a substitution to a non-proline at the amino acid that corresponds to position 251, such for example a substitution to an asparagine (N) corresponding to position 251 and a substitution to a threonine (T) corresponding to position 253. In one embodiment the modified S protein comprises P251N+D253T substitutions or modifications.

Furthermore, the modified S protein may comprise a deletion of one or more than one amino acid corresponding to position 246, 247, 248, 249, 250, 251, 252, or a combination thereof. In one embodiment amino acids corresponding to position 246-252 are deleted and therefore the modified S protein comprises a 246-252 deletion or modification (del246-252). In another embodiment amino acids corresponding to position 247-250 are deleted and therefore the modified S protein comprises a 247-250 deletion or modification (del247-250). In a further embodiment, amino acids corresponding to position 248-251 are deleted and therefore the modified S protein comprises a 248-251 deletion or modification (del248-251). In a further embodiment, amino acids corresponding to position 249-252 are deleted and therefore the modified S protein comprises a 249-252 deletion or modification (del249-252). In another embodiment amino acids corresponding to position 246-250 are deleted and therefore the modified S protein comprises a 246-250 deletion or modification (del246-250). In yet another embodiment amino acids corresponding to position 247-251 are deleted and therefore the modified S protein comprises a 247-251 deletion or modification (del247-251). In another embodiment amino acids corresponding to position 248-252 are deleted and therefore the modified S protein comprises a 248-252 deletion or modification (del248-252). In another embodiment amino acids corresponding to position 246-251 are deleted and therefore the modified S protein comprises a 246-251 deletion or modification (del246-251). In another embodiment amino acids corresponding to position 247-252 are deleted and therefore the modified S protein comprises a 247-252 deletion or modification (del247-252). Non-limiting examples of modified S protein comprising one or more than one deletion are provided in Table 2.

In one embodiment the modified S protein may comprise at least one substitution and the deletion of one or more than one amino acid.

For example, the modified S protein may comprise a substitution to a non-glycine of the amino acid corresponding to position 252 and a deletion of one or more than one amino acids corresponding to position 246, 247, 248, 249, 250, 251, or a combination thereof.

In one embodiment, the modified S protein comprises a substitution to an asparagine (N) corresponding to position 252 and a deletion of amino acids 246-251 and therefore the modified S protein comprises a G252N substitution and a 246-251 deletion (‘G252N+del246-251’ or ‘del246-252+G252N’). In another embodiment, the modified S protein comprises a substitution to an asparagine (N) corresponding to position 252 and a deletion of amino acids 247-250 and therefore the modified S protein comprises a G252N substitution and a 247-250 deletion (‘G252N+del247-250’ or ‘del247-250+G252N’). In another embodiment, the modified S protein comprises a substitution to an asparagine (N) corresponding to position 252 and a deletion of amino acids 248-251 and therefore the modified S protein comprises a G252N substitution and a 248-251 deletion (‘G252N+del248-251’ or ‘del248-251+G252N’). In another embodiment, the modified S protein comprises a substitution to an asparagine (N) corresponding to position 252 and a deletion of amino acids 249-251 and therefore the modified S protein comprises a G252N substitution and a 249-251 deletion (‘G252N+del249-251’ or ‘G252N+del249-251’). In a further embodiment, the modified S protein comprises a substitution to an asparagine (N) corresponding to position 252 and a deletion of amino acids 246-250 and therefore the modified S protein comprises a G252N substitution and a 246-250 deletion (‘G252N+del246-250’ or ‘G252N+del246-250’). In yet another embodiment, the modified S protein comprises a substitution to an asparagine (N) corresponding to position 252 and a deletion of amino acids 247-251 and therefore the modified S protein comprises a G252N substitution and a 247-251 deletion (‘G252N+del247-251’ or ‘G252N+del247-251’). In another embodiment, the modified S protein comprises a substitution to an asparagine (N) corresponding to position 252 and a deletion of amino acids 246-251 and therefore the modified S protein comprises a G252N substitution and a 246-251 deletion (‘G252N+del246-251’ or ‘G252N+del246-251’).

For example, the modified S protein may comprise a substitution to a non-aspartic acid corresponding to position 253 and a deletion of one or more than one amino acids corresponding to position 246, 247, 248, 249, 250, 251, 252, or a combination thereof.

In one embodiment, the modified S protein comprises a substitution to an asparagine (N) corresponding to position 253 and a deletion of amino acids 246-252 and therefore the modified S protein comprises a D253N substitution and a 246-252 deletion (‘D253N+del246-252’ or ‘del246-252+D253N’). In another embodiment, the modified S protein comprises a substitution to an asparagine (N) corresponding to position 253 and a deletion of amino acids 247-250 and therefore the modified S protein comprises a D253N substitution and a 247-250 deletion (‘D253N+del247-250’ or ‘del247-250+D253N’). In another embodiment, the modified S protein comprises a substitution to an asparagine (N) corresponding to position 253 and a deletion of amino acids 248-251 and therefore the modified S protein comprises a D253N substitution and a 248-251 deletion (‘D253N+del248-251’ or ‘del248-251+D253N’). In another embodiment, the modified S protein comprises a substitution to an asparagine (N) corresponding to position 253 and a deletion of amino acids 249-252 and therefore the modified S protein comprises a D253N substitution and a 249-252 deletion (‘D253N+del249-252’ or ‘del249-252+D253N’). In yet another embodiment, the modified S protein comprises a substitution to an asparagine (N) corresponding to position 253 and a deletion of amino acids 246-250 and therefore the modified S protein comprises a D253N substitution and a 246-250 deletion (‘D253N+del246-250’ or ‘del246-250+D253N’). In another embodiment, the modified S protein comprises a substitution to an asparagine (N) corresponding to position 253 and a deletion of amino acids 247-251 and therefore the modified S protein comprises a D253N substitution and a 247-251 deletion (‘D253N+del247-251’ or ‘del247-251+D253N’). In another embodiment, the modified S protein comprises a substitution to an asparagine (N) corresponding to position 253 and a deletion of amino acids 248-252 and therefore the modified S protein comprises a D253N substitution and a 248-252 deletion (‘D253N+del248-252’ or ‘del248-252+D253N’). In another embodiment, the modified S protein comprises a substitution to an asparagine (N) corresponding to position 253 and a deletion of amino acids 246-251 and therefore the modified S protein comprises a D253N substitution and a 246-251 deletion (‘D253N+del246-251’ or ‘del246-251+D253N’). In another embodiment, the modified S protein comprises a substitution to an asparagine (N) corresponding to position 253 and a deletion of amino acids 247-252 and therefore the modified S protein comprises a D253N substitution and a 247-252 deletion (‘D253N+del247-252’ or ‘del247-252+D253N’).

For example, the modified S protein may comprise a substitution to a non-proline of the amino acid corresponding to position 251, a substitution to a non-aspartic acid of the amino acid corresponding to position 253 and a deletion of one or more than one amino acid corresponding to position 246, 247, 248, 249, 250, or a combination thereof.

In one embodiment, the modified S protein comprises a substitution to an asparagine (N) corresponding to position 251, a substitution to an threonine (T) corresponding to position 253 and a deletion of amino acids 246-250 and therefore the modified S protein comprises a P251N substitution, a D253T substitution and a 246-250 deletion (‘P251N+D253T+del246-250’ or ‘del246-250+P251N+D253T’).

In another embodiment, the modified S protein comprises a substitution to an asparagine (N) to position 251, a substitution to an threonine (T) corresponding to position 253 and a deletion of amino acids 247-250 and therefore the modified S protein comprises a P251N substitution, a D253T substitution and a 247-250 deletion (‘P251N+D253T+del247-250’ or ‘del247-250+P251N+D253T’).

In another embodiment, the modified S protein comprises a substitution to an asparagine (N) corresponding to position 251, a substitution to an threonine (T) corresponding to position 253 and a deletion of amino acids 248-250 and therefore the modified S protein comprises a P251N substitution, a D253T substitution and a 248-250 deletion (‘P251N+D253T+del248-250’ or ‘del248-250+P251N+D253T’).

For example the modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 8, 10, 12, 16, 20, 25, 26, 27, 28, 29, 30, 31, 32, 33, 34, 35, 36, 37, 39, 41, 43, 45, 47, 49, 51, 53, 55 or 57, wherein the amino acids corresponding to positions 246-252, 247-250, 248-251, 249-252, 246-250, 247-251, 248-252, 246-251, or 247-252 are deleted, and/or the amino acid corresponding to position 253 is Asparagine (N, Asn), or wherein the amino acid corresponding to positions 247-250, 248-251, 246-250, 247-251 or 246-251 are delete and/or the amino acid corresponding to position 252 is Asparagine (N, Asn), or wherein the amino acid corresponding to positions 247-250 or 246-250 are deleted and/or the amino acid corresponding to position 251 is Asparagine (N, Asn) and position 253 is Threonine (T, Thr), wherein the numbering of positions corresponds to positions in reference sequence SEQ ID NO: 1 and wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 8 or 25, wherein the amino acid corresponding to positions 246-252 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 10, 16, 20, or 26 wherein the amino acid corresponding position 253 is Asparagine (N, Asn), wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 12 or 27 wherein the amino acid corresponding to positions 246-252 are deleted and the amino acid corresponding to position 253 is Asparagine (N, Asn), wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 39 or 28, wherein the amino acid corresponding to position 252 is Asparagine (N, Asn), wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 41 or 29, wherein the amino acid corresponding to position 251 Asparagine (N, Asn) and the amino acid corresponding to position 253 is Threonine (T, Thr), wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 43 or 30, wherein the amino acid corresponding to positions 247-250 are deleted and wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 45 or 31, wherein the amino acid corresponding to positions 248-251 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 47 or 32, wherein the amino acid corresponding to positions 249-252 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 49 or 33, wherein the amino acid corresponding to positions 246-250 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 51 or 34, wherein the amino acid corresponding to positions 247-251 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 53 or 35, wherein the amino acid corresponding to positions 248-252 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 55 or 36, wherein the amino acid corresponding to positions 246-251 are deleted, wherein the modified S protein sequence does not occur naturally and wherein the S proteins when expressed form VLP.

The modified S protein may have an amino acid sequence that has about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 57 or 37, wherein the amino acid corresponding to positions 247-252 are deleted, wherein the modified S protein sequence does not occur naturally and wherein the S proteins when expressed form VLP.

The present specification also provides a nucleic acid comprising a nucleotide sequence encoding a S protein with a substitution corresponding to position 251, 252, 253, deletion corresponding to positions 246-252, 247-250, 248-251, 249-252, 246-250, 247-251, 248-252, 246-251, 247-252, or a substitution corresponding to position 251, 252, 253 and deletion corresponding to positions 246-252, 247-250, 248-251, 249-252, 246-250, 247-251, 248-252, 246-251, 247-252 as described above operatively linked to a regulatory region active in a host or host cell, such as a plant.

Isolation of nucleic acids encoding such S protein is well-known to the one of skill in the art, as is modification of the nucleic acid to introduce changes in the amino acid sequence, e.g., by site-directed mutagenesis.

For example the nucleotide sequences may have about 50, 55, 60, 65, 70, 75, 80, 85, 87, 90, 91, 92, 93 94, 95, 96, 97, 98, 99, 100% or any amount therebetween, sequence identity, with the nucleotide sequence of SEQ ID NO: 9, 11, 15, 19, 38, 40, 42, 44, 46, 48, 50, 52, 54, 56, wherein the nucleotide codon that encode amino acid corresponding to positions 246-252, 247-250, 248-251, 249-252, 246-250, 247-251, 248-252, 246-251, or 247-252 are deleted, and/or the amino acid corresponding to position 253 is Asparagine (N, Asn), or wherein the amino acid corresponding to positions 247-250, 248-251, 246-250, 247-251 or 246-251 are delete and/or the amino acid corresponding to position 252 is Asparagine (N, Asn), or wherein the amino acid corresponding to positions 247-250 or 246-250 are deleted and/or the amino acid corresponding to position 251 is Asparagine (N, Asn) and position 253 is Threonine (T, Thr), wherein the numbering of positions corresponds to positions in reference sequence SEQ ID NO: 1 and wherein the modified S protein sequence does not occur naturally and wherein the S proteins when expressed form VLP.

The modified coronavirus S protein described herewith may be derived from an acceptor S proteins having amino acid sequences about 70, 75, 80, 85, 87, 90, 91, 92, 93, 94, 95, 96, 97, 98, 99, 100% or any amount therebetween, sequence identity, or sequence similarity, with the amino acid sequence of SEQ ID NO: 1, 2, 4, 6, 14 or 18.

Examples of modified S protein proteins having enhanced or improved characteristics as described herewith include, but are not limited to the following:
B CoV S (GSAS-2P+del246-252): Construct: 9801, SEQ ID NO: 8;
B CoV S (GSAS-2P+D253N): Construct: 9802, SEQ ID NO: 10;
B CoV S (GSAS-2P+del246-252+D253N) Construct: 9808, SEQ ID NO: 12;
B.1.617.2 CoV S (GSAS-2P+D253N): Construct: 10011, SEQ ID NO: 16
B.1.1.529 CoV S (GSAS-2P+D253N): Construct: 10092, SEQ ID NO: 20
B CoV S (GSAS-2P+D252N) Construct: 10346, SEQ ID NO: 39;
B CoV S (GSAS-2P+P251N+D253T) Construct: 10351, SEQ ID NO: 41;
B CoV S (GSAS-2P+del247-250) Construct: 10502, SEQ ID NO: 43;
B CoV S (GSAS-2P+del248-251) Construct: 10503, SEQ ID NO: 45;
B CoV S (GSAS-2P+del249-252) Construct: 10504, SEQ ID NO: 47;
B CoV S (GSAS-2P+del246-250) Construct: 10505, SEQ ID NO: 49;
B CoV S (GSAS-2P+del247-251) Construct: 10506, SEQ ID NO: 51;
B CoV S (GSAS-2P+del248-252) Construct: 10507, SEQ ID NO: 53;
B CoV S (GSAS-2P+del246-251) Construct: 10508, SEQ ID NO: 55;
B CoV S (GSAS-2P+del247-252) Construct: 10509, SEQ ID NO: 57.

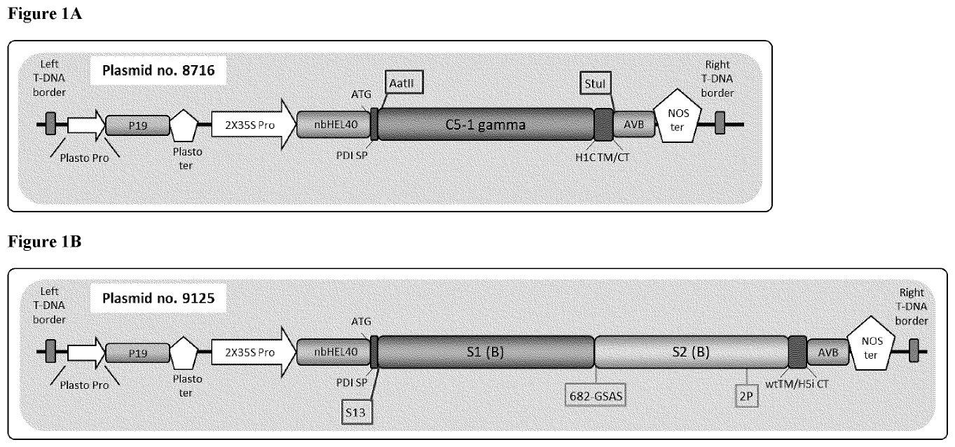

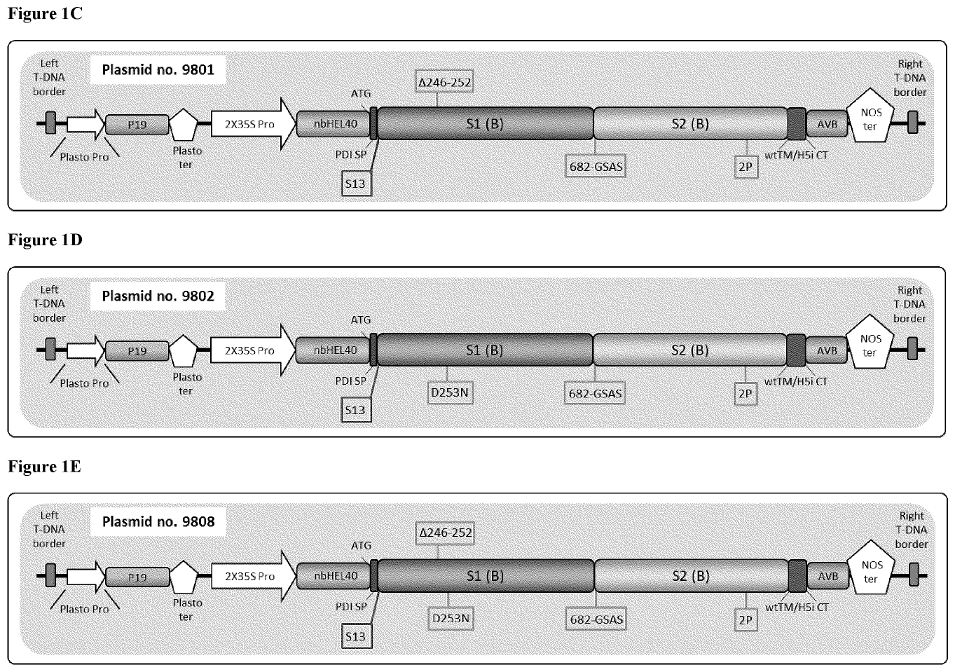

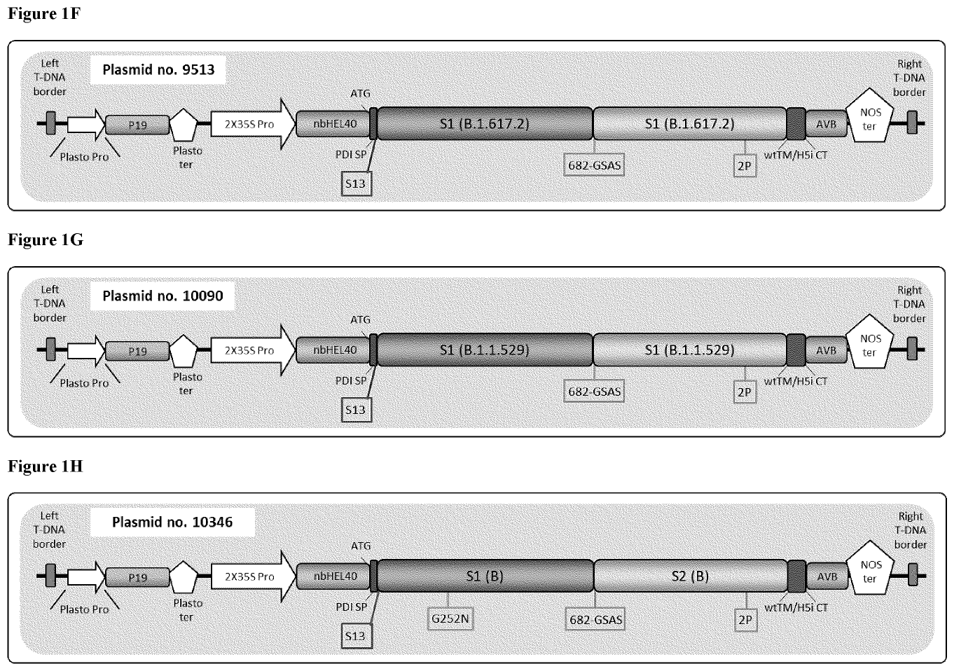

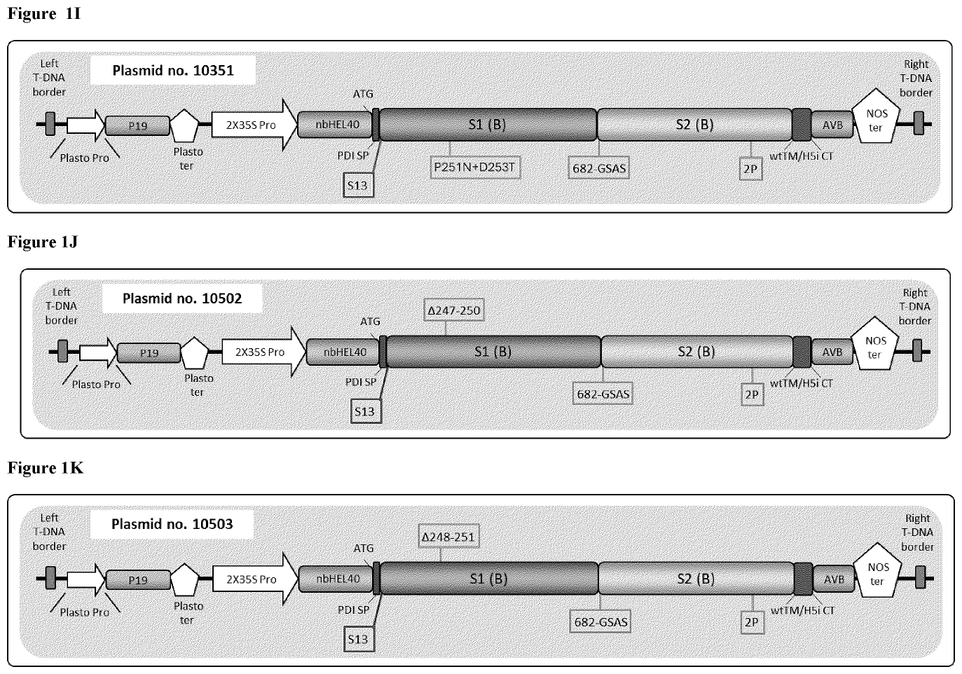

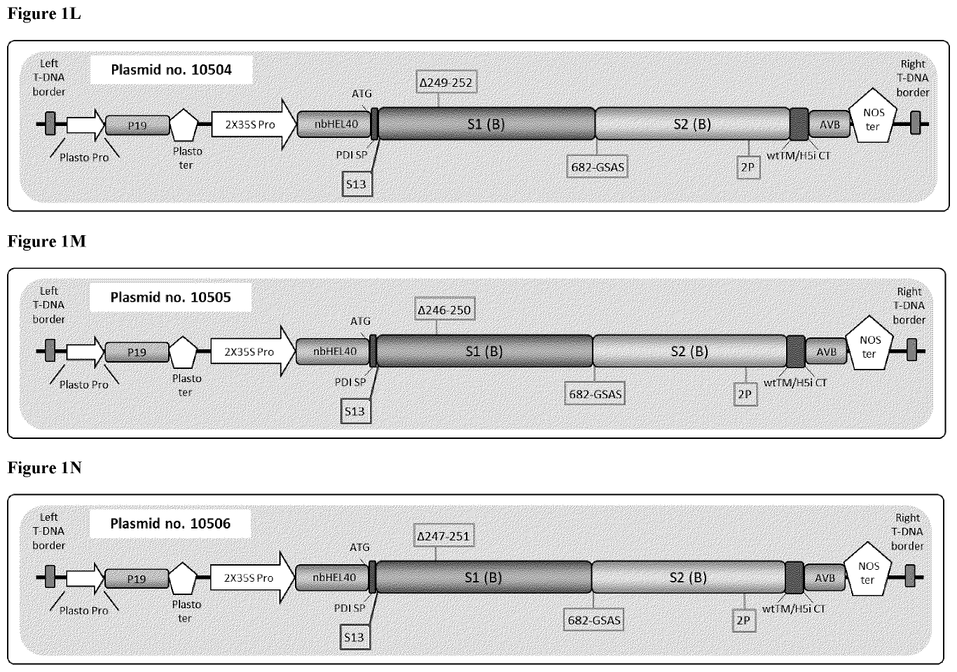

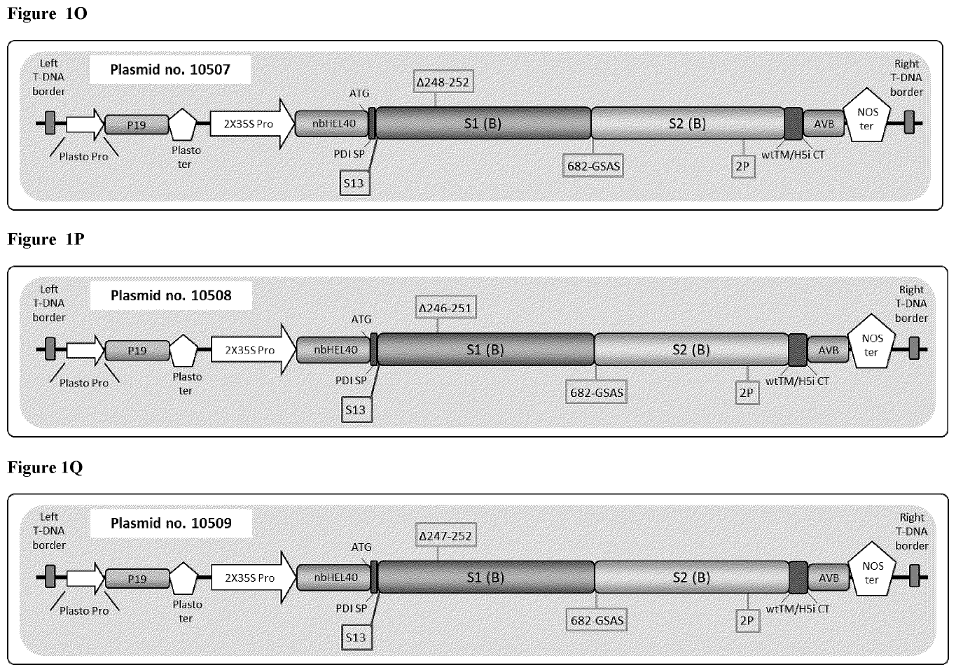

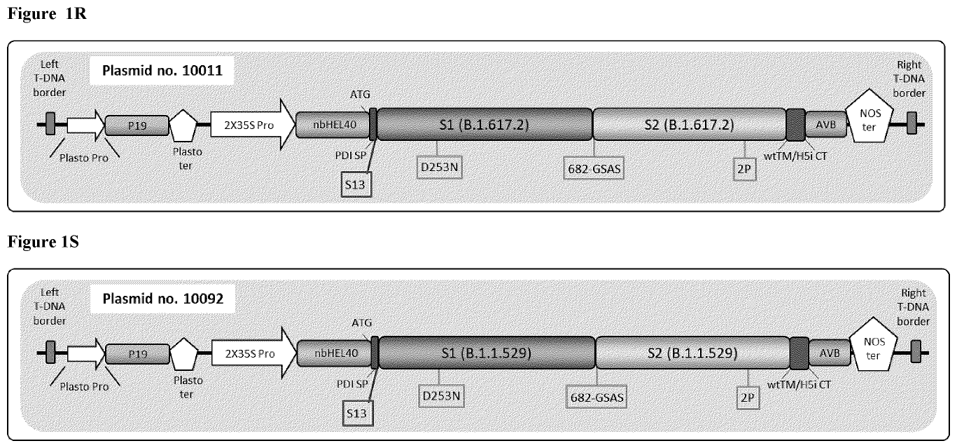

In one embodiment the modified coronavirus S protein may result from an acceptor B lineage SARS-CoV-2 S protein (SEQ ID NO: 3 and 4, construct 9125, FIG. 1B) wherein the del246-252 mutation from the donor C.37 (“Lambda”) lineage has been introduced (SEQ ID NO: 7 and 8, construct 9801, FIG. 1C). In another embodiment the modified coronavirus S protein results from acceptor B lineage SARS-CoV-2 S protein (SEQ ID NO: 3 and 4) wherein the D253N mutation from the donor C.37 (“Lambda”) lineage has been introduced (SEQ ID NO: 9 and 10, construct 9802, FIG. 1D). In another embodiment the modified coronavirus S protein results from acceptor B lineage SARS-CoV-2 S protein (SEQ ID NO: 3 and 4) wherein the del246-252 and D253N mutations from the donor C.37 (“Lambda”) lineage have been introduced (SEQ ID NO: 11 and 12, construct 9808, FIG. 1E). However, as also described herewith, modification that are not found in S protein from a donor lineage may also be introduced into the modified S protein to improved characteristic of the modified S protein compared to the unmodified (parent) S protein.

The modified S protein may be created by introducing changes to the amino acid sequence of the S protein that results in improved characteristic of the modified S protein compared to the unmodified S protein, such as increased stability against proteolysis or increased stability against degradation by proteases, increased integrity, purity and/or homogeneity of the recombinant modified S protein when expressed in a host or host cell.

It is therefore also provided a method of improving the characteristic of a coronavirus S protein, such as for example increased stability against proteolysis or increased stability against degradation by proteases, increased integrity, purity and/or homogeneity of an S protein. The method comprises a) modifying a parent coronavirus S protein to produce a modified coronavirus S protein, wherein the modified coronavirus S protein comprises one or more than one amino acid sequence modification as described above when compared to the parent coronavirus S protein, the one or more than one modification corresponding to amino acids at positions 246, 247, 248, 249, 250, 251, 252, 253, or a combination thereof, of reference sequence SEQ ID NO: 1; and b) expressing the modified coronavirus S protein in a host or host cell, thereby producing a modified S protein with improved characteristics compared to the same characteristics of the parent coronavirus S protein produced under similar conditions in the host or host cell.

It is also provided a method of producing in a host or host cell a modified coronavirus S protein as described herewith, wherein the modified S protein has improved characteristics such as for example increased stability against proteolysis or increased stability against degradation by proteases, increased integrity, purity and/or homogeneity compared to the characteristics of an unmodified S protein. The method comprises a) introducing a nucleic acid encoding a modified S protein as described herewith into the host or host cell, or providing the host or host cell comprising the nucleic acid encoding a modified S protein as described herewith, and b) incubating the host or host cell under conditions that permit the expression of the nucleic acid, thereby producing the modified S protein with improved characteristics compared to the same characteristics of the unmodified S protein produced under similar conditions in the host or host cell. The modified S protein may optionally further be extracted, purified or extracted and purified from the host or host cell.

Furthermore, the modified S protein may assemble into virus-like particle (VLP), wherein content or amount of full-length S protein in the VLP is increased compared to the full-length S protein content of VLP produced from unmodified S protein. It has been observed that by introducing the modifications as described herewith into a coronavirus S protein, the resulting modified S protein is more resistant against proteolysis or clipping of the N-terminal end and higher amounts of full-length S protein may be produced. Accordingly, in another aspect, the disclosure provides VLP comprising modified S protein as described herewith, wherein the VLP comprise an increased content or amount of full-length modified S protein when compared to VLP that comprise unmodified S protein.

The increase of full-length (uncleaved) S protein produced in a host such as plants, may be measured and expressed as an increase in purity of the resulting product. Protein purity is generally assessed by using SDS-PAGE gel and band intensity from SDS PAGE gels can be calculated by densitometry analysis. Briefly, the quantity of recombinant protein species with different molecular weights (i.e. protein bands) produced in the host are determined by densitometry. The protein load is correlated with the peak area of the protein species in the densitometry profile and the peak maximum intensity is used for quantification of the protein species. The purity is calculated as the sum of the relative densities of the protein band(s) of interest expressed in percentage (%) i.e. the more protein(s) of interest are produced, the higher the calculated purity.

Accordingly, the current disclosure further provides a drug substance (DS) comprising, as the desired product, modified S protein as described above, said drug substance being substantially free of product related impurities, wherein the impurities are not immune-active. A preferred drug substance is further substantially free of process related impurities.

By immune-active, it is meant a compound, such as a coronavirus S protein or S protein fragment that is recognized by an antibody specific to the RBD domain of coronavirus S protein (anti-RBD antibody, such for example 40592-T62 from Sino biological) and/or an antibody that is specific to the S2 domain of coronavirus S protein (anti-S2 antibody, such for example NB100-56578 from Novus Biological).

Within the context of the present application, the term “drug substance” refers to a product or active ingredients suitable for use as i) the active principle of a medicament or drug product, ii) an active pharmaceutical ingredient of a medicament or drug product iii) a bulk purified active principle of a medicament or drug product or iv) a bulk purified active ingredient of a medicament or drug product. The medicament or drug product may be a vaccine.

It is therefore further provided a drug substance (DS) comprising a VLP comprising the modified S protein as described herewith. The DS comprising the modified S protein has a higher purity compared to a DS that has been obtained from a host expressing an unmodified S protein. Therefore, it is also provided a method of increasing the purity of a DS obtained from a host or host cell that expresses the modified S protein compared to the purity of a DS that has been obtained from a host that expresses unmodified (parent) S protein.

The modified coronavirus S protein may self-assemble into virus-like particle (VLP). Accordingly, it is also provided a DS comprising VLP comprising modified S protein. The DS comprising VLP with modified S protein exhibits an increased purity compared to a DS that is being produced from the parent (unmodified) coronavirus S protein.

In a further aspect, it is also provided a Drug Product (also referred to as pharmaceutical formulation or pharmaceutical composition). The Drug Product may be formulated as a finished dosage form, for example as a solution, capsule or tablet. The Drug Product comprises the Drug Substance. The Drug Product may further comprise other ingredients such for example pharmaceutically acceptable carriers and/or excipient, such as buffer system, adjuvants, preservatives, tonicity agent(s), chelating agent(s), antiadherents, vehicles etc. Pharmaceutical acceptable carrier and excipient are well known within the art. Therefore, it is also provided a drug product, pharmaceutical formulation or pharmaceutical composition comprising pharmaceutically acceptable carriers and/or excipient, and VLP, the VLP comprising modified S protein or the VLP comprising viral protein, wherein the viral protein consist of modified coronavirus S protein.

### Other Modifications

The modified S protein may be a chimeric modified S protein or a chimeric S protein. By “chimeric S protein”, it is meant a protein or polypeptide that comprises amino acid sequences and/or protein domains or portions of protein domains from two or more than two sources that are fused as a single polypeptide. For example, but not limited to, the ectodomain and the transmembrane domain (TM) or portion of the TM of the chimeric S protein may be derived from a Coronavirus S protein (such as SARS-COV 2), and the cytoplasmic tail (CT) or portion of the CT may be derived from influenza HA (as described in international PCT Application PCT/CA2021/051201, which is herewith incorporated by reference).

The modified S protein may further comprise one or more than one substitution or replacement to stabilize the coronavirus S protein or coronavirus S protein trimer in a prefusion conformation. For example, the modified S protein may further comprise alteration of the consensus RRAR furin cleavage site and two consecutive proline substitutions at or near the boundary between a HR1 domain and a central helix domain that stabilize the S ectodomain trimer in the prefusion conformation, as described for example in WO 2018/081318 and PCT/CA2021/051201, which are herein incorporated by reference.

### VLP

Modified coronavirus S protein as described herewith may further be incorporated into virus-like particles (VLPs). The term virus-like particle” (VLP), or “virus-like particles” or “VLPs” refers to virus-like structures that are generally morphologically and antigenically similar to virions produced in an infection, but lack genetic information sufficient to replicate and thus are non-infectious. VLPs are structures that self-assemble and comprise one or more structural proteins such as for example modified coronavirus S protein. Therefore, the VLP may comprise modified coronavirus S protein. VLP may further comprise coronavirus protein, wherein the coronavirus protein consists of modified coronavirus S protein.

As shown in FIGS. 2B-2D, when expressed in N. benthamiana, modified B lineage S proteins (constructs: 9801, 9802 and 9808) were observed to form VLPs similar to the unmodified B lineage SARS-CoV-2 S protein (FIG. 2A, construct 9125). Expression of S protein from the donor C.37 (“Lambda”) lineage (FIG., 2E, construct 9588) similarly formed VLPs like the acceptor B lineage SARS-CoV-2 S protein.

As further shown in FIG. 6C, when expressed in N. benthamiana, modified B lineage S proteins (constructs: 9802, 10346 and 10351) were observed to form VLPs similar to the unmodified B lineage SARS-CoV-2 S protein (FIG. 2A, construct 9125).

VLP may be produced in suitable host or host cells including plants and plant cells. Following extraction from the host or host cell and upon isolation and further purification under suitable conditions, VLP may be recovered as intact structures.

The VLP may be purified or extracted using any suitable method for example chemical or biochemical extraction. VLPs are relatively sensitive to desiccation, heat, pH, surfactants and detergents. Therefore it may be useful to use methods that maximize yields, minimize contamination of the VLP fraction with cellular proteins, maintain the integrity of the proteins, or VLP, and, where required, the associated lipid envelope or membrane, methods of loosening the cell wall to release the proteins, or VLP. Minimizing or eliminating the use of detergents or surfactants such for example SDS or Triton™ X-100 may be beneficial for improving the yield of VLP extraction. VLP may be then assessed for structure and size by, for example, electron microscopy (see FIG. 4B), or by size exclusion chromatography.

For enveloped viruses, such as Coronavirus, it may be advantageous for a lipid layer or membrane to be retained by the virus. The composition, quality and quantity of the lipid may vary with the system (e.g. a plant-produced enveloped virus would include plant lipids or phytosterols in the envelope), and may contribute to an improved immune response.

Therefore, the VLP that are produced in a host or host cell, may comprise lipids from the plasma membrane of the host or host cell. For example VLP produced in plants may contain lipids of plant origin (“plant lipids”), VLP produced in insect cells may comprise lipids from the plasma membrane of insect cells (generally referred to as “insect lipids”), and VLP produced in mammalian cells may comprise lipids from the plasma membrane of mammalian cells (generally referred to as “mammalian lipids”).

The plant lipids or plant-derived lipids may be in the form of a lipid bilayer, and may further comprise an envelope surrounding the VLP. The plant-derived lipids may comprise lipid components of the plasma membrane of the plant where the VLP is produced, including phospholipids, tri-, di- and monoglycerides, as well as fat-soluble sterol or metabolites comprising sterols. Examples include phosphatidylcholine (PC), phosphatidylethanolamine (PE), phosphatidylinositol, phosphatidylserine, glycosphingolipids, phytosterols or a combination thereof. Examples of phytosterols include campesterol, stigmasterol, ergosterol, brassicasterol, delta-7-stigmasterol, delta-7-avenasterol, daunosterol, sitosterol, 24-methylcholesterol, cholesterol or beta-sitosterol. As one of skill in the art would understand, the lipid composition of the plasma membrane of a cell may vary with the culture or growth conditions of the cell or organism, or species, from which the cell is obtained. Generally, beta-sitosterol is the most abundant phytosterol.

Without wishing to be bound by theory, plant-made VLP comprising plant derived lipids, may induce a stronger immune reaction than VLP made in other manufacturing systems and the immune reaction induced by these plant-made VLP may be stronger when compared to the immune reaction induced by live or attenuated whole virus vaccines.

Furthermore, in addition to the potential adjuvant effect of the presence of plant lipids, the ability of plant N-glycans to facilitate the capture of glycoprotein antigens by antigen presenting cells, may be advantageous of the production of VLP in plants.

The VLP produced within a plant may comprise a modified S protein comprising plant-specific N-glycans. Therefore, this disclosure also provides for a VLP comprising modified S protein having plant specific N-glycans. Furthermore, it is provided VLP comprising plant lipids and modified S protein having plant specific N-glycans.

### Methods

Methods of producing a modified S protein, or a virus like particle (VLP) comprising modified S protein in a host or host cell are also provided.

The methods comprise the introduction of nucleic acid comprising a sequence that encodes a modified S protein as described herewith into a host or host cell, or providing the host or host cell comprising the nucleic acid encodes a modified S protein as described herewith, and incubating the host or host cell under conditions that permit the expression of the nucleic acid, thereby producing the modified S protein. The modified S protein may then self-assemble into VLP that comprise the modified S protein (see FIGS. 2B-2D and 6C).

In addition, it is provided methods of increasing the purity or homogeneity of coronavirus S protein s produced in a host or host cell. The methods comprise the introduction nucleic acid comprising a sequence that encodes a modified S protein as described herewith into a host or host cell, or providing the host or host cell comprising the nucleic acid encodes a modified S protein as described herewith, and incubating the host or host cell under conditions that permit the expression of the nucleic acid, thereby producing the modified S protein, wherein the produced S protein have an increased purity and/or homogeneity compared to the purity and/or homogeneity of unmodified S protein s produced under similar conditions in the host or host cell.

It is further provided a modified S protein produced by the method as described herewith, wherein the modified S protein has increased purity and/or homogeneity compared to the purity and/or homogeneity of an unmodified S protein produced under similar conditions in a host or host cell.

The modified S protein as described herewith may have purity and/or homogeneity of about between 80%-98% or any amount therebetween. For example the modified S protein may have a purity and/or homogeneity of about 80, 85, 90, 91, 92, 93, 94, 95, 96, 97, 98% or any amount therebetween.

It is desirable that recombinant protein samples are pure and homogeneous, i.e. that the recombinant protein exist throughout the sample in the same consistent form and size.

In the current application, by “increased purity” or “increased homogeneity” it is meant an increase of recombinant S protein in a stable and uniform protein length, for example the full length of the expressed protein. As discussed above, during expression in a host cell or subsequent purification from the host cell, a recombinant protein may be degraded (also referred to as “clipped”, “hydrolyzed” or “truncated”) at one or more residues by enzymatic, proteolytic, or chemical events subsequent or concurrent with expression or translation. Without wishing to be bound by theory, degradation of most proteins may be attributed to host cell-derived proteases.

As described in the current application, when the S protein is modified, a more stable and homogenous protein sample is obtained. More specifically, it has been found that when the modified S protein is produced in a host or host cell, such as a plant or plant cells, less of cleaved or truncated version of the S protein are observed, and that the modified S protein exhibits an increase in stability against proteolysis, compared to an unmodified S protein sample.

By modifying the S protein as described herewith, the proportion of cleaved, truncated or clipped S protein is reduced and the proportion of full-length S protein is increased. Accordingly, the modified S protein incorporating modifications as described herewith may result in reduced proportion of cleaved, truncated or clipped S protein and increased proportion of full-length S protein relative to unmodified S protein, when produced in a host or host cell. For example the ratio between a full-length modified S protein to truncated modified S protein is between about 1:0.02 to 1:0.25 or any amount therebetween of full-length to truncated modified S protein ratio, for example 1:0.05, 1:0.1, 1:0.15, 1:2 or 1:0.25 of full-length to truncated modified S protein ratio.

With reference to the coronavirus S protein or modified coronavirus S protein, “full length S protein” (also referred to as complete S protein) or “full length modified S protein” (also referred to as complete modified S protein) refers to an S protein or modified S protein that has a polypeptide sequence and/or length that corresponds to the theoretical polypeptide sequence or length of the construct from which the recombinant S protein or recombinant modified S protein is expressed. The full-length S protein or modified S protein may include a signal peptide to direct localization when expressed in the host or host cell. The signal peptide may be a native (with respect to the protein) signal or leader sequence, or a heterologous signal sequence.

Therefore, as described herein, the modified S protein may be produced as precursor protein comprising a modified S-protein and a heterologous amino acid signal peptide sequence. For example, the modified S protein precursor may comprise the signal peptide from Protein disulphide isomerase (PDI SP; nucleotides 32-103 of Accession No. Z11499).

Since the signal peptide is cleaved when the mature S protein is produced in a host or host cell, full-length modified S protein may therefore also refer to the mature modified S protein without the signal peptide. The full length S protein or modified S protein is in contrast to a truncated S protein, wherein portions of the N- or C-terminal of the mature S protein may have been eliminated by proteolysis.

For example, the full-length S protein may correspond to the full length of the sequences of SEQ ID NO: 4, 6, 8, 10, 12, 14, 16, 18, or 20, wherein the sequences may include a signal peptide. Alternatively, the full-length S protein may correspond to the full length of the sequences of SEQ ID NO: 4, 6, 8, 10, 12, 14, 16, 18, 20, 39, 41, 43, 45, 47, 49, 51, 53, 55 or 57 without the signal peptide. For example the full-length modified S protein may correspond to the full length of the sequences of SEQ ID NO: 25, 26, 27, 28, 29, 30, 31, 32, 33, 34, 35, 36, or 37.

With reference to the S protein or modified S protein, “cleaved S protein”, “truncated S protein”, “clipped S protein” or “cleaved modified S protein”, “truncated modified S protein” or “clipped modified S protein” refers to an expressed polypeptide of the coronavirus S protein which is cut or truncated at one or more residues by enzymatic, proteolytic, or chemical events subsequent or concurrent with expression or translation, wherein the cleavage or truncation is a result of unintended proteolytic processing of the protein. “Cleaved S protein”, “truncated S protein”, “clipped S protein”, “cleaved modified S protein”, “truncated modified S protein” or “clipped modified S protein” does not include an S protein (or modified S protein), wherein the signal peptide has been cleaved to produce the mature S protein or mature modified S protein.

The present disclosure also relates to methods of improving N-terminal homogeneity of a coronavirus S protein (modified S protein), wherein 80%, 85%, 90%, 91%, 92%, 93%, 94%, 95%, 96%, 97%, 98% or 99% of the coronavirus S protein with modifications correct N-terminal amino acid sequence.

One or more than one modified genetic constructs comprising the modified S protein of the present description may be expressed in any suitable host or host cell that is transformed by the nucleic acids, or nucleotide sequence, or constructs, or vectors of the present disclosure. The host or host cell may be from any source including plants, fungi, bacteria, insect and animals for example mammals. The host may further be a non-human host. For example, the host or host cell may be selected from a plant or plant cell, a fungi or a fungi cell, a bacteria or bacteria cell, an insect or an insect cell, and animal or an animal cell. The mammal or animal may not be a human. In a preferred embodiment the host or host cell is a plant, portion of a plant or plant cell.

The term “plant”, “portion of a plant”, “plant portion’, “plant matter”, “plant biomass”, “plant material”, plant extract”, or “plant leaves”, as used herein, may comprise an entire plant, plant cell, tissue, cells, or any fraction thereof, intracellular plant components, extracellular plant components, liquid or solid extracts of plants, or a combination thereof, that are capable of providing the transcriptional, translational, and post-translational machinery for expression of one or more than one nucleic acids described herein, and/or from which an expressed protein or VLP may be extracted and purified. Plants may include, but are not limited to, herbaceous plants. The herbaceous plants may be annuals, biennials or perennials plants. Plants may further include, but are not limited to agricultural crops including for example canola, Brassica spp., maize, Nicotiana spp., (tobacco) for example, Nicotiana benthamiana, Nicotiana rustica, Nicotiana, tabacum, Nicotiana alata, Arabidopsis thaliana, alfalfa, potato, sweet potato (Ipomoea batatus), ginseng, pea, oat, rice, soybean, wheat, barley, sunflower, cotton, corn, rye (Secale cereale), sorghum (Sorghum bicolor, Sorghum vulgare), safflower (Carthamus tinctorius).

The term “plant portion”, as used herein, refers to any part of the plant including but not limited to leaves, stem, root, flowers, fruits, a plant cell obtained from leaves, stem, root, flowers, fruits, a plant extract obtained from leaves, stem, root, flowers, fruits, or a combination thereof. In one embodiment the plant portion refers to the areal portion of a plant such as for example leaves, stem, flowers and fruits. The term “plant extract”, as used herein, refers to a plant-derived product that is obtained following treating a plant, a portion of a plant, a plant cell, or a combination thereof, physically (for example by freezing followed by extraction in a suitable buffer), mechanically (for example by grinding or homogenizing the plant or portion of the plant followed by extraction in a suitable buffer), enzymatically (for example using cell wall degrading enzymes), chemically (for example using one or more chelators or buffers), or a combination thereof. A plant extract may be further processed to remove undesired plant components for example cell wall debris. A plant extract may be obtained to assist in the recovery of one or more components from the plant, portion of the plant or plant cell, for example a protein (including protein complexes, protein surprastructures and/or VLP), a nucleic acid, a lipid, a carbohydrate, or a combination thereof from the plant, portion of the plant, or plant cell. If the plant extract comprises proteins, then it may be referred to as a protein extract. A protein extract may be a crude plant extract, a partially purified plant or protein extract, or a purified product, that comprises one or more proteins, protein complexes such for example protein trimers, protein suprastructures, and/or VLP, from the plant tissue. If desired a protein extract, or a plant extract, may be partially purified using techniques known to one of skill in the art, for example, the extract may be subjected to salt or pH precipitation, centrifugation, gradient density centrifugation, filtration, chromatography, for example, size exclusion chromatography, ion exchange chromatography, affinity chromatography, or a combination thereof. A protein extract may also be purified, using techniques that are known to one of skill in the art.

The constructs of the present disclosure can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. DT. Dennis, DH Turpin, DD Lefebvre, DB Layzell (eds), Addison Wesly, Langmans Ltd. London, pp. 561-579 (1997). Other methods include direct DNA uptake, the use of liposomes, electroporation, for example using protoplasts, micro-injection, microprojectiles or whiskers, and vacuum infiltration. See, for example, Bilang, et al. (Gene 100:247-250 (1991), Scheid et al. (Mol. Gen. Genet. 228:104-112, 1991), Guerche et al. (Plant Science 52:111-116, 1987), Neuhause et al. (Theor. Appl Genet. 75:30-36, 1987), Klein et al., Nature 327:70-73 (1987); Howell et al. (Science 208:1265, 1980), Horsch et al. (Science 227:1229-1231, 1985), DeBlock et al., Plant Physiology 91:694-701, 1989), Methods for Plant Molecular Biology (Weissbach and Weissbach, eds., Academic Press Inc., 1988), Methods in Plant Molecular Biology (Schuler and Zielinski, eds., Academic Press Inc., 1989), Liu and Lomonossoff (J Virol Meth, 105:343-348, 2002,), U.S. Pat. Nos. 4,945,050; 5,036,006; and 5,100,792, U.S. patent application Ser. No. 08/438,666, filed May 10, 1995, and Ser. No. 07/951,715, filed Sep. 25, 1992, (all of which are hereby incorporated by reference).

As described below, transient expression methods may be used to express the constructs of the present disclosure (see Liu and Lomonossoff, 2002, Journal of Virological Methods, 105:343-348; which is incorporated herein by reference). Alternatively, a vacuum-based transient expression method, as described by Kapila et al., 1997, which is incorporated herein by reference) may be used. These methods may include, for example, but are not limited to, a method of Agro-inoculation or Agroinfiltration, syringe infiltration, however, other transient methods may also be used as noted above. With Agro-inoculation, Agroinfiltration, or syringe infiltration, a mixture of Agrobacteria comprising the desired nucleic acid enter the intercellular spaces of a tissue, for example the leaves, aerial portion of the plant (including stem, leaves and flower), other portion of the plant (stem, root, flower), or the whole plant. After crossing the epidermis the Agrobacteria infect and transfer t-DNA copies into the cells. The t-DNA is episomally transcribed and the mRNA translated, leading to the production of the protein of interest in infected cells, however, the passage of t-DNA inside the nucleus is transient.

To aid in identification of transformed plant cells, the constructs of this disclosure may be further manipulated to include plant selectable markers. Useful selectable markers include enzymes that provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (beta-glucuronidase), or luminescence, such as luciferase or GFP, may be used.

Also considered part of this disclosure are transgenic plants, plant cells or seeds containing the gene construct of the present disclosure that may be used as a platform plant suitable for transient protein expression described herein. Methods of regenerating whole plants from plant cells are also known in the art (for example see Guerineau and Mullineaux (1993, Plant transformation and expression vectors. In: Plant Molecular Biology Labfax (Croy R R D ed) Oxford, BIOS Scientific Publishers, pp 121-148). In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques. Transgenic plants can also be generated without using tissue culture. Methods for stable transformation, and regeneration of these organisms are established in the art and known to one of skill in the art. Available techniques are reviewed in Vasil et al. (Cell Culture and Somatic Cell Genetics of Plants, Vol I, II and III, Laboratory Procedures and Their Applications, Academic Press, 1984), and Weissbach and Weissbach (Methods for Plant Molecular Biology, Academic Press, 1989). The method of obtaining transformed and regenerated plants is not critical to the present disclosure.

If plants, plant portions or plant cells are to be transformed or co-transformed by two or more nucleic acid constructs, the nucleic acid construct may be introduced into the Agrobacterium in a single transfection event so that the nucleic acids are pooled, and the bacterial cells transfected. Alternatively, the constructs may be introduced serially. In this case, a first construct is introduced into the Agrobacterium as described, the cells are grown under selective conditions (e.g. in the presence of an antibiotic) where only the singly transformed bacteria can grow. Following this first selection step, a second nucleic acid construct is introduced into the Agrobacterium as described, and the cells are grown under double-selective conditions, where only the double-transformed bacteria can grow. The double-transformed bacteria may then be used to transform a plant, portion of the plant or plant cell as described herein, or may be subjected to a further transformation step to accommodate a third nucleic acid construct.

Alternatively, if plants, plant portions, or plant cells are to be transformed or co-transformed by two or more nucleic acid constructs, the nucleic acid construct may be introduced into the plant by co-infiltrating a mixture of Agrobacterium cells with the plant, plant portion, or plant cell, each Agrobacterium cell may comprise one or more constructs to be introduced within the plant. In order to vary the relative expression levels within the plant, plant portion or plant cell, of a nucleotide sequence of interest within a construct, during the step of infiltration, the concentration of the various Agrobacteria populations comprising the desired constructs may be varied.

A nucleic acid sequence or nucleotide sequence referred to in the present disclosure, may be “substantially homologous”, “substantially similar” or “substantially identical” to a sequence, or a compliment of the sequence if the nucleic acid sequence or nucleotide sequence hybridise to one or more than one nucleotide sequence or a compliment of the nucleic acid sequence or nucleotide sequence as defined herein under stringent hybridisation conditions. Sequences are “substantially homologous” “substantially similar” “substantially identical” when at least about 60%, or between 60 to 100%, or any amount therebetween, for example 60, 62, 64, 66, 68, 70, 72, 74, 76, 78, 80, 82, 84, 86, 88, 90, 92, 94, 96, 98, 100%, or any amount therebetween, of the nucleotides match over a defined length of the nucleotide sequence providing that such homologous sequences exhibit one or more than one of the properties of the sequence, or the encoded product as described herein.

The terms “percent similarity”, “sequence similarity”, “percent identity”, or “sequence identity”, when referring to a particular sequence, are used for example as set forth in the University of Wisconsin GCG software program, or by manual alignment and visual inspection (see, e.g., Current Protocols in Molecular Biology, Ausubel et al., eds. 1995 supplement). Methods of alignment of sequences for comparison are well-known in the art. Optimal alignment of sequences for comparison can be conducted, using for example the algorithm of Smith & Waterman, (1981, Adv. Appl. Math. 2:482), by the alignment algorithm of Needleman & Wunsch, (1970, J. Mol. Biol. 48:443), by the search for similarity method of Pearson & Lipman, (1988, Proc. Natl. Acad. Sci. USA 85:2444), by computerized implementations of these algorithms (for example: GAP, BESTFIT, FASTA, and TFASTA in the Wisconsin Genetics Software Package, Genetics Computer Group (GCG), 575 Science Dr., Madison, Wis.) or by manual alignment and visual inspection (see, e.g., Current Protocols in Molecular Biology (Ausubel et al., eds. 1995 supplement)).

An example of an algorithm suitable for determining percent sequence identity and sequence similarity are the BLAST and BLAST 2.0 algorithms, which are described in Altschul et al., (1977, Nuc. Acids Res. 25:3389-3402) and Altschul et al., (1990, J. Mol. Biol. 215:403-410), respectively. BLAST and BLAST 2.0 are used, with the parameters described herein, to determine percent sequence identity for the nucleic acids and proteins of the disclosure. For example the BLASTN program (for nucleotide sequences) may use as defaults a wordlength (W) of 11, an expectation (E) of 10, M=5, N=−4 and a comparison of both strands. For amino acid sequences, the BLASTP program may use as defaults a word length of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands. Software for performing BLAST analyses is publicly available through the National Center for Biotechnology Information (see URL: ncbi.nlm.nih.gov/).

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. The process of optimizing the nucleotide sequence coding for a heterologously expressed protein may be an important step for improving expression yields. The optimization requirements may include steps to improve the ability of the host to produce the foreign protein.

There are different codon-optimization techniques known in the art for improving, the translational kinetics of translationally inefficient protein coding regions. These techniques mainly rely on identifying the codon usage for a certain host organism. If a certain gene or sequence should be expressed in this organism, the coding sequence of such genes and sequences will then be modified such that one will replace codons of the sequence of interest by more frequently used codons of the host organism.

“Codon optimization” is defined as modifying a nucleic acid sequence for enhanced expression in a host or host cell of interest by replacing at least one, more than one, or a significant number, of codons of the native sequence with codons that may be more frequently or most frequently used in the genes of another organism or species. Various species exhibit particular bias for certain codons of a particular amino acid.

The present disclosure includes synthetic polynucleotide sequences that have been codon optimized for example the sequences have been optimized for human codon usage or plant codon usage. The codon optimized polynucleotide sequences may then be expressed in the host for example plants. More specifically the sequences optimized for human codon usage or plant codon usage may be expressed in plants. Without wishing to be bound by theory, it is believed that the sequences optimized for human codon increases the guanine-cytosine content (GC content) of the sequence and improves expression yields when plants are used as host.

The term “construct”, “vector” or “expression vector”, as used herein, refers to a recombinant nucleic acid for transferring exogenous nucleotide sequences (for example a nucleotide sequences encoding the modified S protein as described herewith) into host cells (e.g. plant cells) and directing expression of the exogenous nucleic acid sequences in the host cells. “Expression cassette” refers to a nucleic acid comprising a nucleotide sequence of interest under the control of, and operably (or operatively) linked to, an appropriate promoter or other regulatory elements for transcription of the nucleic acid of interest in a host cell. As one of skill in the art would appreciate, the expression cassette may comprise a termination (terminator) sequence that is any sequence that is active the host cell (e.g. plant host). For example in plants, the termination sequence may be derived from the RNA-2 genome segment of a bipartite RNA virus, e.g. a comovirus, the termination sequence may be a NOS terminator, or terminator sequence may be obtained from the 3′UTR of the alfalfa plastocyanin gene.

The nucleic acid comprising a nucleotide sequence encoding a modified S protein, as described herein may further comprise sequences that enhance expression of the S protein in the host, portion of the host or host cell. Sequences that enhance expression may include, a 5′ UTR enhancer element, or a plant-derived expression enhancer, in operative association with the nucleic acid encoding the modified viral structural protein. The sequence encoding the modified S protein may also be optimized to increase expression by for example optimizing for human codon usage, increased GC content, or a combination thereof.

By “regulatory region” “regulatory element” or “promoter” it is meant a portion of nucleic acid typically, but not always, upstream of the protein coding region of a gene, which may be comprised of either DNA or RNA, or both DNA and RNA. When a regulatory region is active, and in operative association, or operatively linked, with a nucleotide sequence of interest, this may result in expression of the nucleotide sequence of interest. A regulatory element may be capable of mediating organ specificity, or controlling developmental or temporal gene activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element may comprise a basal promoter element, responsible for the initiation of transcription, as well as other regulatory elements that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well. Examples of tissue-specific regulatory regions, for example see-specific a regulatory region, include the napin promoter, and the cruciferin promoter (Rask et al., 1998, J. Plant Physiol. 152:595-599; Bilodeau et al., 1994, Plant Cell 14:125-130). An example of a leaf-specific promoter includes the plastocyanin promoter (see U.S. Pat. No. 7,125,978, which is incorporated herein by reference).

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form, which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P., 1998, Trends Plant Sci. 3, 352-358). Examples, of potential inducible promoters include, but not limited to, tetracycline-inducible promoter (Gatz, C., 1997, Ann. Rev. Plant Physiol. Plant Mol. Biol. 48, 89-108), steroid inducible promoter (Aoyama, T. and Chua, N. H., 1997, Plant J. 2, 397-404) and ethanol-inducible promoter (Salter, M. G., et al, 1998, Plant Journal 16, 127-132; Caddick, M. X., et al, 1998, Nature Biotech. 16, 177-180) cytokinin inducible IB6 and CKI1 genes (Brandstatter, I. and Kieber, J. J., 1998, Plant Cell 10, 1009-1019; Kakimoto, T., 1996, Science 274, 982-985) and the auxin inducible element, DR5 (Ulmasov, T., et al., 1997, Plant Cell 9, 1963-1971).

A constitutive regulatory region directs the expression of a gene throughout the various parts of a plant and continuously throughout plant development. Examples of known constitutive regulatory elements include promoters associated with the CaMV 35S transcript. (p35S; Odell et al., 1985, Nature, 313:810-812; which is incorporated herein by reference), the rice actin 1 (Zhang et al, 1991, Plant Cell, 3:1155-1165), actin 2 (An et al., 1996, Plant J., 10:107-121), or tms 2 (U.S. Pat. No. 5,428,147), and triosephosphate isomerase 1 (Xu et. al., 1994, Plant Physiol. 106:459-467) genes, the maize ubiquitin 1 gene (Cornejo et al, 1993, Plant Mol. Biol. 29:637-646), the Arabidopsis ubiquitin 1 and 6 genes (Holtorf et al, 1995, Plant Mol. Biol. 29:637-646), the tobacco translational initiation factor 4A gene (Mandel et al, 1995 Plant Mol. Biol. 29:995-1004), the Cassava Vein Mosaic Virus promoter, pCAS, (Verdaguer et al., 1996); the promoter of the small subunit of ribulose biphosphate carboxylase, pRbcS: (Outchkourov et al., 2003), the pUbi (for monocots and dicots).

The term “constitutive” as used herein does not necessarily indicate that a nucleotide sequence under control of the constitutive regulatory region is expressed at the same level in all cell types, but that the sequence is expressed in a wide range of cell types even though variation in abundance is often observed.

One or more of the genetic constructs of the present disclosure may also include further enhancers, either translation or transcription enhancers, as may be required. Enhancers may be located 5′ or 3′ to the sequence being transcribed. Enhancer regions are well known to persons skilled in the art, and may include an ATG initiation codon, adjacent sequences or the like. The initiation codon, if present, may be in phase with the reading frame (“in frame”) of the coding sequence to provide for correct translation of the transcribed sequence.

The term “5′UTR” or “5′ untranslated region”, “5′ leader sequence” or “5′ UTR enhancer element” refers to regions of an mRNA that are not translated. The 5′UTR typically begins at the transcription start site and ends just before the translation initiation site or start codon of the coding region. The 5′ UTR may modulate the stability and/or translation of an mRNA transcript.

The term “plant-derived expression enhancer”, as used herein, refers to a nucleotide sequence obtained from a plant, the nucleotide sequence encoding a 5′UTR. Examples of a plant derived expression enhancer are described in U.S. Provisional Patent Application No. 62/643,053 (Filed Mar. 14, 2018) and International Application No. PCT/CA2019/050319 (Filed Mar. 14, 2019); which are incorporated herein by reference) or in Diamos A. G. et al. (2016, Front Plt Sci. 7:1-15; which is incorporated herein by reference). The plant-derived expression enhancer may be selected from nbEPI42, nbSNS46, nbCSY65, nbHEL40, nbSEP44, nbMT78, nbATL75, nbDJ46, nbCHP79, nbEN42, atHSP69, atGRP62, atPK65, atRP46, nb30S72, nbGT61, nbPV55, nbPPI43, nbPM64 and nbH2A86 as described in U.S. 62/643,053 and PCT/CA2019/050319. The plant derived expression enhancer may be used within a plant expression system comprising a regulatory region that is operatively linked with the plant-derived expression enhancer sequence and a nucleotide sequence of interest, for example a nucleotide sequence encoding a modified S protein.

Stability and/or translation efficiency of an RNA may further be improved by the inclusion of a 3′ untranslated region (3′UTR). The one or more genetic constructs of the present description may therefore further comprise a 3′ UTR.

A 3′ untranslated region may contain a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′ AATAAA-3′ although variations are not uncommon. Non-limiting examples of suitable 3′ regions are the 3′ transcribed non-translated regions containing a polyadenylation signal of Agrobacterium tumor inducing (Ti) plasmid genes, such as the nopaline synthase (Nos gene) and plant genes such as the soybean storage protein genes, the small subunit of the ribulose-1, 5-bisphosphate carboxylase gene (ssRUBISCO; U.S. Pat. No. 4,962,028; which is incorporated herein by reference), the promoter used in regulating plastocyanin expression, described in U.S. Pat. No. 7,125,978 (which is incorporated herein by reference), 3′ UTR derived from a Arracacha virus B isolate gene (AvB), 3′UTR derived from Beet necrotic yellow vein virus (trBNYVV), 3′UTR derived from Southern bean mosaic virus (SBMV), 3′UTR derived from Turnip ringspot virus (TuRSV), 3′ UTR derived from Cowpea Mosaic Virus (CPMV), 3′UTR derived from Broad bean true mosaic virus (BBTMV) or 3′UTR derived from Ourmia melon virus (trOUMV). The 3′UTR might be used in conjunction with 5′UTR derived from heterologous sequences to modulate expression levels.

It is therefore provided a “construct”, “vector”, “expression vector” or “expression cassette” that comprises a nucleic acid comprising a nucleotide sequence of interest (such as a modified viral structural protein) under the control of, and operably (or operatively) linked to a 3′UTR. Furthermore, the nucleic acid may comprise a 3′UTR operably (or operatively) linked to a nucleotide sequence of interest (such as a modified viral structural protein).

The modified S protein or VLP comprising a modified S protein as described herewith, may be used to elicit an immune response in a subject.

An “immune response” generally refers to a response of the adaptive immune system of a subject. The adaptive immune system generally comprises a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. The terms “modulate” or “modulation” or the like refer to an increase or decrease in a particular response or parameter, as determined by any of several assays generally known or used, some of which are exemplified herein.

A cell-mediated response is an immune response that does not involve antibodies but rather involves the activation of macrophages, natural killer cells (NK), antigen-specific cytotoxic T-lymphocytes, and the release of various cytokines in response to an antigen. Cell-mediated immunity is used generally to refer to some Th cell activation, Tc cell activation and T-cell mediated responses. Cell mediated immunity may be of particular importance in responding to viral infections.

For example, the induction of antigen specific CD8 positive T lymphocytes may be measured using an ELISPOT assay; stimulation of CD4 positive T-lymphocytes may be measured using a proliferation assay. Anti-Coronavirus antibody titers may be quantified using an ELISA assay; isotypes of antigen-specific or cross-reactive antibodies may also be measured using anti-isotype antibodies (e.g. anti-IgG, IgA, IgE or IgM). Methods and techniques for performing such assays are well-known in the art.

Cytokine presence or levels may also be quantified. For example a T-helper cell response (Th1/Th2) will be characterized by the measurement of IFN-γ and IL-4 secreting cells using by ELISA (e.g. BD Biosciences OptEIA kits). Peripheral blood mononuclear cells (PBMC) or splenocytes obtained from a subject may be cultured, and the supernatant analyzed. T lymphocytes may also be quantified by fluorescence-activated cell sorting (FACS), using marker specific fluorescent labels and methods as are known in the art.

A microneutralization assay may also be conducted to characterize an immune response in a subject, see for example the methods of Rowe et al., 1973. Virus neutralization titers may be quantified in a number of ways, including: enumeration of lysis plaques (plaque assay) following crystal violent fixation/coloration of cells; microscopic observation of cell lysis in in vitro culture; and ELISA and spectrophotometric detection of Coronavirus.

The term “epitope” or “epitopes”, as used herein, refers to a structural part of an antigen to which an antibody specifically binds.

A method of producing an antibody or antibody fragment is provided, the method comprises administering the modified S protein, a trimer or trimeric modified S protein, VLP, composition or vaccine comprising the modified S protein as described herewith to a subject, or a host animal, thereby producing the antibody or the antibody fragment. Antibodies or the antibody fragments produced by the method are also provided.

The present disclosure therefore also provides the use of a modified coronavirus S protein or VLP comprising the modified coronavirus S protein, as described herein, for inducing immunity to a Coronavirus infection in a subject. Also disclosed herein is an antibody or antibody fragment, prepared by administering the modified coronavirus S protein or VLP comprising the modified coronavirus S protein, to a subject or a host animal.

Further provided is a composition comprising an effective dose of modified coronavirus S protein or VLP comprising the modified coronavirus S protein, as described herein, and a pharmaceutically acceptable carrier, adjuvant, vehicle, or excipient, for inducing an immune response in a subject. Also provided is a vaccine for inducing an immune response again Coronavirus in a subject, wherein the vaccine comprises an effective dose of the modified coronavirus S protein or VLP comprising the modified coronavirus S protein.

The composition or vaccine may comprise VLP comprising the modified S protein from one type of Coronavirus family, sub-group, type, subtype, lineage, subgenera or strain, or the composition or vaccine may comprise multiple VLP types, wherein each VLP type comprises modified S protein, wherein the modified S proteins in the same VLP are derived from one type of Coronavirus family, sub-group, type, subtype, lineage, subgenera or strain i.e. the composition or vaccine may comprise a mixture of different Coronavirus VLP, wherein each VLP may comprise a modified S protein from the same Coronavirus family, sub-group, type, subtype, lineage, subgenera or strain. For example the composition or vaccine may comprise a first VLP comprising a first modified S protein from a first Coronavirus family, sub-group, type, subtype, lineage or strain and a second VLP comprising a second modified S protein from a second Coronavirus family, sub-group, type, subtype, lineage or strain. Furthermore the composition may also comprise a third VLP comprising a third modified S protein from a third Coronavirus family, sub-group, type, subtype, lineage or strain and/or the composition or vaccine may comprise a fourth VLP comprising a fourth modified S protein from a fourth Coronavirus family, sub-group, type, subtype, lineage, subgenera or strain.

The composition or vaccine may further comprise VLP comprising modified S protein from more than one type of Coronavirus family, sub-group, type, subtype, lineage, subgenera or strain. For example the VLP may comprise a first modified S protein from a first Coronavirus family, sub-group, type, subtype, lineage or strain and a second modified S protein from a second Coronavirus family, sub-group, type, subtype, lineage or strain. Furthermore the VLP may comprise a third modified S protein from a third Coronavirus family, sub-group, type, subtype, lineage or strain and/or the VLP may comprise a fourth modified S protein from a fourth Coronavirus family, sub-group, type, subtype, lineage, subgenera or strain.

Accordingly, the description also provides compositions or vaccines that are monovalent (univalent), or multivalent (polyvalent). The monovalent composition or vaccine may immunize a subject against a single type of Coronavirus strain, whereas the multivalent composition or vaccine may immunize a subject against more than one Coronavirus strain. For example, the composition or vaccine may be a bivalent composition or vaccine, which upon administration, may immunize a subject against two different types of Coronavirus families, sub-groups, types, subtypes, lineages or strains. Furthermore, the composition or vaccine may be a trivalent composition, or the vaccine or composition may be a tetravalent or quadrivalent composition or vaccine. Furthermore, the vaccine may also be multivalent with respect to different types of viruses. For example the vaccine may immunize a subject against one or more than one Coronavirus strain (first type of virus) and against a second type of virus for example influenza virus.

The monovalent or multivalent composition or vaccine with may further comprise a pharmaceutically acceptable carrier, adjuvant, vehicle, or excipient, for inducing an immune response in a subject.

Adjuvant systems to enhance a subject's immune response to a vaccine antigen are well known and may be used in conjunction with the vaccine or pharmaceutical composition as described herewith. There are many types of adjuvants that may be used. Common adjuvants for human use are aluminum hydroxide, aluminum phosphate and calcium phosphate. There are also a number of adjuvants based on oil emulsions (oil in water or water in oil emulsions such as Freund's incomplete adjuvant (FIA), Montanide™, Adjuvant 65, and Lipovant™), products from bacterial (or their synthetic derivatives), endotoxins, fatty acids, paraffinic, or vegetable oils, cholesterols, and aliphatic amines or natural organic compounds such for example squalene. Non-limiting adjuvants that might be used include for example oil-in water emulsions of squalene oil (for example MF-59 or AS03), adjuvant composed of the synthetic TLR4 agonist glucopyranosyl lipid A (GLA) integrated into stable emulsion (SE) (GLA-SE) or CpG 1018 a toll-like receptor (TLR9) agonist adjuvant.

Therefore the vaccine or pharmaceutical composition may comprise one or more than one adjuvant. For example the vaccine or pharmaceutical composition may comprise aluminum hydroxide, aluminum phosphate, calcium phosphate, an oil in water or water in oil emulsions, an emulsion comprising squalene (for example MF-59 or AS03), an emulsion comprising GLA-SE, or CpG 1018 adjuvant. In one embodiment the adjuvant is AS03.

The pharmaceutical compositions, vaccines or formulations of the present description may be manufactured in a manner that is itself known, e.g., by means of conventional mixing, dissolving, granulating, dragee-making, levigating, emulsifying, encapsulating, entrapping or tableting processes.

The pharmaceutical compositions, vaccines or formulations may be produced by mixing or premixing of any constituent components before administration, for example by manual or mechanically aided mixing of two or more vaccine suspensions, pharmaceutically acceptable carriers, adjuvants, vehicles, or excipients as a step performed before the final formulation, vaccine, or pharmaceutical composition is administered.

The pharmaceutical compositions, vaccines or formulations may be administered to a subject orally, intradermally, intranasally, intramuscularly, intraperitoneally, intravenously, or subcutaneously.

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

The composition or vaccine may be administered to a subject once (single dose). Furthermore, the vaccine or composition may be administered to a subject multiple times (multi-dose). Therefore the composition, formulation, or vaccine may be administered to a subject in a single dose to illicit an immune response or the composition, formulation, or vaccine may be administered multiple time (multi dosages). For example a dose of the composition or vaccine may be administered 2, 3, 4 or 5 times. Accordingly, the composition or vaccine may be administered to a subject in an initial dose and one or more than one doses may subsequently be administered to the subject. Administration of the doses may be separated in time from each other. For example after the administration of an initial dose, one or more than one subsequent dose may be administered 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 month or any time in between from the administration of the initial dose. Furthermore, the composition or vaccine may be administered annually. For example the composition or vaccine may be administered as a seasonal vaccine.

The disclosure further provides the following sequences.

TABLE 1
SEQ ID NOs and Description of Sequences

| SEQ ID | |
|---|---|
| NO: | Description of Sequence |
| 1 | Native B lineage SARS-CoV-2 S protein wtTM/CT AA (P0DTC2) |
| 2 | Native B lineage SARS-CoV-2 S protein wtTM/CT AA (P0DTC2) without signal peptide (SP) |
| 3 | B CoV S (GSAS-2P) DNA |
| 4 | B CoV S (GSAS-2P) AA |
| 5 | C.37 CoV S (GSAS-2P) DNA |
| 6 | C.37 CoV S (GSAS-2P) AA |
| 7 | B CoV S (GSAS-2P + de1246-252) DNA |
| 8 | B CoV S (GSAS-2P + del246-252) AA |
| 9 | B CoV S (GSAS-2P + D253N) DNA |
| 10 | B CoV S (GSAS-2P + D253N) AA |
| 11 | B CoV S (GSAS-2P + del246-252 + D253N) DNA |
| 12 | B CoV S (GSAS-2P + del246-252 + D253N) AA |
| 13 | B.1.617.2 CoV S (GSAS-2P) DNA |
| 14 | B.1.617.2 CoV S (GSAS-2P) AA |
| 15 | B.1.617.2 CoV S (GSAS-2P + D253N) DNA |
| 16 | B.1.617.2 CoV S (GSAS-2P + D253N) AA |
| 17 | B.1.1.529 CoV S (GSAS-2P) DNA |
| 18 | B.1.1.529 CoV S (GSAS-2P) AA |
| 19 | B.1.1.529 CoV S (GSAS-2P + D253N) DNA |
| 20 | B.1.1.529 CoV S (GSAS-2P + D253N) AA |
| 21 | Cloning vector 8716 from left to right T-DNA |
| 22 | Construct 9125 from 2X35S prom to NOS term |
| 23 | IF(PDI)S-3-NY-CoV.c |
| 24 | IF(Avb)-H5I.r |
| 25 | Mature B CoV S (GSAS-2P + S13 + del246-252) AA without SP |
| 26 | Mature B CoV S (GSAS-2P + S13 + D253N) AA without SP |
| 27 | Mature B CoV S (GSAS-2P ++ S13del246-252 + D253N) AA without SP |
| 28 | Mature B CoV S (GSAS-2P + S13 + G252N) AA without SP |
| 29 | Mature B CoV S (GSAS-2P + S13 + P251N + D253T) AA without SP |
| 30 | Mature B CoV S (GSAS-2P + S13 + Δ247-250) AA without SP |
| 31 | Mature B CoV S (GSAS-2P + S13 + Δ248-251) AA without SP |
| 32 | Mature B CoV S (GSAS-2P + S13 + Δ249-252) AA without SP |
| 33 | Mature B CoV S (GSAS-2P + S13 + Δ246-250) AA without SP |
| 34 | Mature B CoV S (GSAS-2P + S13 + Δ247-251) AA without SP |
| 35 | Mature B CoV S (GSAS-2P + S13 + Δ248-252) AA without SP |
| 36 | Mature B CoV S (GSAS-2P + S13 + Δ246-251) AA without SP |
| 37 | Mature B CoV S (GSAS-2P + S13 + Δ247-252) AA without SP |
| 38 | B CoV S (GSAS-2P + S13 + G252N) DNA |
| 39 | B CoV S (GSAS-2P + S13 + G252N) AA |
| 40 | B CoV S (GSAS-2P + S13 + P251N + D253T) DNA |
| 41 | B CoV S (GSAS-2P + S13 + P251N + D253T) AA |
| 42 | B CoV S (GSAS-2P + S13 + Δ247-250) DNA |
| 43 | B CoV S (GSAS-2P + S13 + Δ247-250) AA |
| 44 | B CoV S (GSAS-2P + S13 + Δ248-251) DNA |
| 45 | B CoV S (GSAS-2P + S13 + Δ248-251) AA |
| 46 | B CoV S (GSAS-2P + S13 + Δ249-252) DNA |
| 47 | B CoV S (GSAS-2P + S13 + Δ249-252) AA |
| 48 | B CoV S (GSAS-2P + S13 + Δ246-250) DNA |
| 49 | B CoV S (GSAS-2P + S13 + Δ246-250) AA |
| 50 | B CoV S (GSAS-2P + S13 + Δ247-251) DNA |
| 51 | B CoV S (GSAS-2P + S13 + Δ247-251) AA |
| 52 | B CoV S (GSAS-2P + S13 + Δ248-252) DNA |
| 53 | B CoV S (GSAS-2P + S13 + Δ248-252) AA |
| 54 | B CoV S (GSAS-2P + S13 + Δ246-251) DNA |
| 55 | B CoV S (GSAS-2P + S13 + Δ246-251) AA |
| 56 | B CoV S (GSAS-2P + S13 + Δ247-252) DNA |
| 57 | B CoV S (GSAS-2P + S13 + 4Δ247-252) AA |

The present invention will be further illustrated in the following examples.

### EXAMPLES

### Example 1: Constructs

### Modified Coronavirus S Protein (Constructs Number 9125, 9588, 9801, 9802, 9808, 9513, 10011, 10090, 10092, 10346, 10351, 10502, 10503, 10504, 10505, 10506, 10507, 10508, and 10509) in 2X35S-nbHEL40/AvB-NOS Term Expression System.

A sequence encoding Spike(S) protein from B lineage SARS-CoV-2 hCoV-19/USA/CA2/2020 in which the native signal peptide has been replaced by that of alfalfa protein disulfide isomerase (PDISP/CoV S) was cloned into 2X35S/nbHEL40-AvB/NOS expression system using the following PCR-based method. A fragment containing the PDISP/CoV S coding sequence was amplified using primers IF (PDI)S-3-NY-CoV.c (SEQ ID NO: 23) and IF (Avb)-H5I.r (SEQ ID NO: 24), using PDISP/CoV S sequence (SEQ ID NO: 3) as template. The PCR product was cloned into 2X35S/nbHEL40-AvB/NOS expression system using In-Fusion cloning system (Clontech, Mountain View, CA). Construct number 8716 (FIG. 1A) was digested with AatII and StuI restriction enzymes and the linearized plasmid was used for the In-Fusion assembly reaction. Construct number 8716 is an acceptor plasmid intended for “In Fusion” cloning of genes of interest in a 2X35S/nbHEL40-AvB/NOS-based expression cassette. This acceptor plasmid also incorporates a gene construct for the co-expression of the TBSV P19 suppressor of silencing under the alfalfa Plastocyanin gene promoter and terminator. The backbone is a pCAMBIA binary plasmid and the sequence from left to right t-DNA borders is shown in SEQ ID NO: 21. The resulting construct was given number 9125 (SEQ ID NO: 22). The amino acid sequence of complete B lineage SARS-CoV-2 Spike fused with PDISP is presented in SEQ ID NO: 4. A representation of plasmid 9125 is presented in FIG. 1B.

TABLE 2
Summary of deletion constructs. The numbering of amino acidresidues
corresponds to the position in reference sequence of SEQ ID NO: 1.

| | | | | | | | |
|---|---|---|---|---|---|---|---|
| 246 | 247 | 248 | 249 | 250 | 251 | 252 | Construct |

| ------------------------------------------------------------------------------- | | #9801 |
|---|---|---|
| | ------------------------------------------ | #10502 |

| | |
|---|---|
| ------------------------------------------- | #10503 |

| | |
|---|---|
| -------------------------------------------- | #10504 |

| | |
|---|---|
| ------------------------------------------------------ | #10505 |

| | |
|---|---|
| ------------------------------------------------------ | #10506 |

| | |
|---|---|
| -------------------------------------------------------- | #10507 |

| | |
|---|---|
| ------------------------------------------------------------------ | #10508 |

| | |
|---|---|
| ------------------------------------------------------------------- | #10509 |

Constructs 9588, 9801, 9802, 9808, 9513, 10011, 10090, 10092, 10346, 10351, 10502, 10503, 10504, 10505, 10506, 10507, 10508, and 10509 were cloned using the same methodology as for plasmid 9125 and a summary of primers, templates, accepting vectors and products is provided in Table 3 below.

TABLE 3

| | | | Primer 1 |
|---|---|---|---|
| | | | (forward |
| Construct | | TMCT | primer of |
| # | Gene of interest (GOI) | Region | fragment 1) |
| 9125 | B CoV S (GSAS-2P) | wtTM/H5iCT | SEQ ID NO: 23 |
| 9588 | C.37 CoV S (GSAS-2P) | wtTM/H5iCT | SEQ ID NO: 23 |
| 9801 | B CoV S (GSAS-2P + del246-252) | wtTM/H5iCT | SEQ ID NO: 23 |
| 9802 | B CoV S (GSAS-2P + D253N) | wtTM/H5iCT | SEQ ID NO: 23 |
| 9808 | B CoV S (GSAS-2P + del246-252 + D253N) | wtTM/H5iCT | SEQ ID NO: 23 |
| 9513 | B.1.617.2 CoV S (GSAS-2P) | wtTM/H5iCT | SEQ ID NO: 23 |
| 10011 | B.1.617.2 CoV S (GSAS-2P + D253N) | wtTM/H5iCT | SEQ ID NO: 23 |
| 10090 | B.1.1.529 CoV S (GSAS-2P) | wtTM/H5iCT | SEQ ID NO: 23 |
| 10092 | B.1.1.529 CoV S (GSAS-2P + D253N) | wtTM/H5iCT | SEQ ID NO: 23 |
| 10346 | B CoV S (GSAS-2P + S13 + G252N) | wtTM/H5iCT | SEQ ID NO: 23 |
| 10351 | B CoV S (GSAS-2P + S13 + P251N + D253T) | wtTM/H5iCT | SEQ ID NO: 23 |
| 10502 | B CoV S (GSAS-2P + S13 + Δ247-250) | wtTM/H5iCT | SEQ ID NO: 23 |
| 10503 | B CoV S (GSAS-2P + S13 + Δ248-251) | wtTM/H5iCT | SEQ ID NO: 23 |
| 10504 | B CoV S (GSAS-2P + S13 + Δ249-252) | wtTM/H5iCT | SEQ ID NO: 23 |
| 10505 | B CoV S (GSAS-2P + S13 + Δ246-250) | wtTM/H5iCT | SEQ ID NO: 23 |
| 10506 | B CoV S (GSAS-2P + S13 + Δ247-251) | wtTM/H5iCT | SEQ ID NO: 23 |
| 10507 | B CoV S (GSAS-2P + S13 + Δ248-252) | wtTM/H5iCT | SEQ ID NO: 23 |
| 10508 | B CoV S (GSAS-2P + S13 + Δ246-251) | wtTM/H5iCT | SEQ ID NO: 23 |
| 10509 | B CoV S (GSAS-2P + S13 + Δ247-252) | wtTM/H5iCT | SEQ ID NO: 23 |

| | Primer 2 | Template | | |
|---|---|---|---|---|
| | (reverse | for first | | |
| Construct | primer of | PCR/Resulting | Resulting | Acceptor |
| # | fragment 1) | gene | protein | plasmid |
| 9125 | SEQ ID NO: 24 | SEQ ID NO: 3 | SEQ ID NO: 4 | 8716 (AatII-StuI) |
| 9588 | SEQ ID NO: 24 | SEQ ID NO: 5 | SEQ ID NO: 6 | 8716 (AatII-StuI) |
| 9801 | SEQ ID NO: 24 | SEQ ID NO: 7 | SEQ ID NO: 8 | 8716 (AatII-StuI) |
| 9802 | SEQ ID NO: 24 | SEQ ID NO: 9 | SEQ ID NO: 10 | 8716 (AatII-StuI) |
| 9808 | SEQ ID NO: 24 | SEQ ID NO: 11 | SEQ ID NO: 12 | 8716 (AatII-StuI) |
| 9513 | SEQ ID NO: 24 | SEQ ID NO: 13 | SEQ ID NO: 14 | 8716 (AatII-StuI) |
| 10011 | SEQ ID NO: 24 | SEQ ID NO: 15 | SEQ ID NO: 16 | 8716 (AatII-StuI) |
| 10090 | SEQ ID NO: 24 | SEQ ID NO: 17 | SEQ ID NO: 18 | 8716 (AatII-StuI) |
| 10092 | SEQ ID NO: 24 | SEQ ID NO: 19 | SEQ ID NO: 20 | 8716 (AatII-StuI) |
| 10346 | SEQ ID NO: 24 | SEQ ID NO: 38 | SEQ ID NO: 39 | 8716 (AatII-StuI) |
| 10351 | SEQ ID NO: 24 | SEQ ID NO: 40 | SEQ ID NO: 41 | 8716 (AatII-StuI) |
| 10502 | SEQ ID NO: 24 | SEQ ID NO: 42 | SEQ ID NO: 43 | 8716 (AatII-StuI) |
| 10503 | SEQ ID NO: 24 | SEQ ID NO: 44 | SEQ ID NO: 45 | 8716 (AatII-StuI) |
| 10504 | SEQ ID NO: 24 | SEQ ID NO: 46 | SEQ ID NO: 47 | 8716 (AatII-StuI) |
| 10505 | SEQ ID NO: 24 | SEQ ID NO: 48 | SEQ ID NO: 49 | 8716 (AatII-StuI) |
| 10506 | SEQ ID NO: 24 | SEQ ID NO: 50 | SEQ ID NO: 51 | 8716 (AatII-StuI) |
| 10507 | SEQ ID NO: 24 | SEQ ID NO: 52 | SEQ ID NO: 53 | 8716 (AatII-StuI) |
| 10508 | SEQ ID NO: 24 | SEQ ID NO: 54 | SEQ ID NO: 55 | 8716 (AatII-StuI) |
| 10509 | SEQ ID NO: 24 | SEQ ID NO: 56 | SEQ ID NO: 57 | 8716 (AatII-StuI) |

### Example 2: Methods

Agrobacterium tumefaciens Transfection

Agrobacterium tumefaciens strain AGL1 was transfected by electroporation with the SARS-CoV-2 modified S protein expression vectors using the methods described by D'Aoust et al., 2008 (Plant Biotech. J. 6:930-40).

### Preparation of Plant Biomass, Inoculum and Agroinfiltration

N. benthamiana plants were grown from seeds in flats filled with a peat moss substrate. The plants were allowed to grow in the greenhouse under a controlled photoperiod and temperature regime. Three weeks after seeding, individual plantlets were picked out, transplanted in pots and left to grow in the greenhouse for three additional weeks under the same environmental conditions.

Agrobacteria transfected with each expression vector were grown until they reached an OD₆₀₀ between 0.6 and 1.6. Agrobacterium suspensions were centrifuged before use and resuspended in infiltration medium (10 mM MgCl₂ and 10 mM MES pH 5.6) and stored overnight at 4° C. On the day of infiltration, culture batches were diluted in 2.5 culture volumes and allowed to warm before use. Whole plants of N. benthamiana were placed upside down in the bacterial suspension in an air-tight stainless steel tank under a vacuum for 2-min. Plants were returned to the greenhouse for a 6 to 9-day incubation period until harvest.

### Leaf Harvest and Total Protein and VLP Extraction

Following incubation, the aerial part of plants was harvested, frozen at −80° C. and crushed into pieces. Total soluble proteins were extracted by mechanically homogenizing (Polytron) each sample of frozen-crushed plant material in two volumes of cold 50 mM Tris buffer at pH 8.0+500 mM NaCl, 0.4 μg/ml metabisulfite and 1 mM phenylmethanesulfonyl fluoride. After homogenization, the slurries were centrifuged at 10,000 g for 10 min at 4° C. and these clarified crude extracts (supernatant) kept for analysis.

In planta complete S protein percentage were assessed on clarified crude extracts and analyzed using a capillary-based electrophoresis method (Protein Simple, BioTechne) technology and a WES analysis system. In brief, soluble proteins from crude extracts were separated by molecular weight in a capillary and fixed to the matrix. Anti-S2 antibody (Novus biological, cat #NB100-56578) is used for detection according to the manufacturer instructions. For this method, two bands (the full S protein and a smaller fragment) are recognized by the anti-S2 antibody. Complete S proportion is measured to evaluate the % of complete protein. In planta complete S protein proportion of coronavirus S protein and modified coronavirus S protein are depicted in FIGS. 3A, 3B, 6B and 7.

### Electron Microscopy

To determine whether expressed S protein assembled into VLPs, transmission electron microscopy (TEM) of immuno-trapped particles was performed on purified VLPs. Glow discharged carbon/copper grids (10 s, 0.3 mbar) were placed on 20 μL of purified VLPs (100 μg/mL) for 5 min and then washed 4 times with sterile distilled water. The grids were floated on 20 μL of 2% uranyl acetate for 1 min, excess solution is then removed by touching a moist filter paper and allowed to dry for 24 h on a filter paper before viewing under a TEM (Tecnai Microscope). TEM images of VLP formed by coronavirus S protein and modified coronavirus S protein are shown in FIGS. 2A-2E and FIG. 6C, as further described in the present application.

### Example 3: Complete S Protein Assessment

Complete S protein percentage (%) in planta was assessed by capillary Western Blot with the crude extract as described above with detection of modified coronavirus S protein using an antibody and quantification using a standard curve. In planta proportion of full-length coronavirus S protein versus cleaved coronavirus S protein (“purity” or “Complete S protein (%)”) are shown for coronavirus S protein and modified coronavirus S protein in FIGS. 3A, 3B, 6B and 7 as further described in the present application.

Drug substance (DS) purity (%) was assessed after small-scale clarification and purification to remove the impurities by densitometry analysis of coomassie-stained protein on SDS gel and immunologically relevant products are included in quantification and purity measurement. Drug substance (DS) purity (%) are shown for coronavirus S protein and modified coronavirus S protein in FIGS. 4A and 4B, as further described in the present application.

### Example 4: Stability Assessment of S Protein

The modified S protein and unmodified S protein were either extracted using enzyme extraction methods at pH5.5 (Enz. pH5.5) or pH 6.1 (Enz. pH6.1) or by mechanical extraction method. (Mech) (FIG. 5). For enzymatic digestion, diced fresh leaves were incubated in plastic containers with a pectinase mix in 200 mM mannitol+125 mM citrate+500 mM NaCl+25 mM EDTA+0.04% (W/V) bisulfite (pH 6.2) and pH was adjusted to 5.5 or kept at 6.1 for 5.25 hours at 20° C. For mechanical extraction, diced fresh biomass was extracted using 2 volumes of the same buffer in a blender for two minutes and pH was adjusted at 5.5. The extracts were clarified using 400 μm mesh and centrifuged 15 minutes at 4500 g at 4° C. Clarified extracts were then incubated 20 hours at 20° C. and complete S protein percentage were assessed before and after the overnight incubation using the method previously described.

### Example 5: Sequences

Native SARS-CoV-2 S protein wtTM/CT AA (PODTC2)
SEQ ID NO: 1
MFVFLVLLPLVSSQCVNLTTRTQLPPAYTNSFTRGVYYPDKVFRSSVLHSTQDLFLPFF
SNVTWFHAIHVSGTNGTKRFDNPVLPENDGVYFASTEKSNIIRGWIFGTTLDSKTQSLL
IVNNATNVVIKVCEFQFCNDPFLGVYYHKNNKSWMESEFRVYSSANNCTFEYVSQPFLM
DLEGKQGNFKNLREFVFKNIDGYFKIYSKHTPINLVRDLPQGFSALEPLVDLPIGINIT
RFQTLLALHRSYLTPGDSSSGWTAGAAAYYVGYLQPRTELLKYNENGTITDAVDCALDP
LSETKCTLKSFTVEKGIYQTSNERVQPTESIVRFPNITNLCPFGEVENATRFASVYAWN
RKRISNCVADYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKPFERDISTEI
YQAGSTPCNGVEGENCYFPLQSYGFQPTNGVGYQPYRVVVLSFELLHAPATVCGPKKST
NLVKNKCVNFNFNGLTGTGVLTESNKKELPFQQFGRDIADTTDAVRDPQTLEILDITPC
SFGGVSVITPGTNTSNQVAVLYQDVNCTEVPVAIHADQLTPTWRVYSTGSNVFQTRAGC
LIGAEHVNNSYECDIPIGAGICASYQTQTNSPRRARSVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLENKV
TLADAGFIKQYGDCLGDIAARDLICAQKENGLTVLPPLLTDEMIAQYTSALLAGTITSG
WTFGAGAALQIPFAMQMAYRENGIGVTQNVLYENQKLIANQFNSAIGKIQDSLSSTASA
LGKLQDVVNQNAQALNTLVKQLSSNFGAISSVLNDILSRLDKVEAEVQIDRLITGRLQS
LQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTF
VSGNCDVVIGIVNNTVYDPLQPELDSFKEELDKYFKNHTSPDVDLGDISGINASVVNIQ
KEIDRLNEVAKNLNESLIDLQELGKYEQYIKWPWYIWLGFIAGLIAIVMVTIMLCCMTS
CCSCLKGCCSCGSCCKFDEDDSEPVLKGVKLHYT
Native SARS-CoV-2 S protein wtTM/CT AA (PODTC2) without
signal peptide (SP)
SEQ ID NO: 2
QCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNEKNLR
EFVFKNIDGYFKIYSKHTPINLVRDLPQGFSALEPLVDLPIGINITRFQTLLALHRSYL
TPGDSSSGWTAGAAAYYVGYLQPRTELLKYNENGTITDAVDCALDPLSETKCTLKSFTV
EKGIYQTSNERVQPTESIVRFPNITNLCPFGEVENATRFASVYAWNRKRISNCVADYSV
LYNSASFSTFKCYGVSPTKLNDLCFTNVYADSFVIRGDEVRQIAPGQTGKIADYNYKLP
DDFTGCVIAWNSNNLDSKVGGNYNYLYRLFRKSNLKPFERDISTEIYQAGSTPCNGVEG
FNCYFPLQSYGFQPTNGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTN
TSNQVAVLYQDVNCTEVPVAIHADQLTPTWRVYSTGSNVFQTRAGCLIGAEHVNNSYEC
DIPIGAGICASYQTQTNSPRRARSVASQSIIAYTMSLGAENSVAYSNNSIAIPTNFTIS
VTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGD
CLGDIAARDLICAQKENGLTVLPPLLTDEMIAQYTSALLAGTITSGWTFGAGAALQIPF
AMQMAYRFNGIGVTQNVLYENQKLIANQFNSAIGKIQDSLSSTASALGKLQDVVNQNAQ
ALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQEKNE
TTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNL
NESLIDLQELGKYEQYIKWPWYIWLGFIAGLIAIVMVTIMLCCMTSCCSCLKGCCSCGS
CCKFDEDDSEPVLKGVKLHYT
B CoV S (GSAS-2P) DNA
SEQ ID NO: 3
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatcctatctgacacccggagactcctctagcggctggactgccggcgctgccg
cttattacgttggttatcttcagccacgcacgttcctgctgaagtataacgagaatggt
actattaccgatgccgtggattgtgcccttgaccccctgtccgaaactaagtgcacact
caagtcattcactgtggaaaaaggaatctaccagacaagcaattttcgggtccagccta
ctgagagcattgtgcgctttcctaacatcacaaatctttgccccttcggagaggttttc
aatgctacacggtttgcctccgtgtatgcctggaaccgcaagagaatttccaattgcgt
ggccgattactccgtgctctacaatagtgcaagctttagcacctttaagtgctatggcg
tatcccctactaagcttaacgacttgtgtttcacaaacgtgtatgccgactcctttgtg
atacggggcgacgaagttagacagatagcaccaggacagacgggaaagatagctgacta
caactataagcttcctgatgacttcactggctgcgttatcgcgtggaattctaacaacc
tggactcaaaagtcggcggcaactataactatctctatcggctgttccgcaagagtaac
cttaagccctttgagagagatataagcactgaaatctaccaggctggcagtacgccctg
taatggcgtggaaggctttaattgttattttccactgcaatcctatggttttcagccaa
ccaatggcgtgggctaccaaccataccgcgtcgtggtgctctcctttgaactgctccac
gctcccgcgactgtctgcggccccaagaagtccacgaaccttgtgaagaataagtgcgt
taattttaatttcaacggcctcactggaacaggagtgctcactgagagtaacaagaagt
tcctgccatttcaacaatttggcagagacatagccgatactactgacgccgttagggac
ccccagaccctcgagattctcgatataacgccctgctccttcggtggagtttccgtgat
cacgccaggcaccaataccagtaaccaggtcgccgtgctgtatcaggatgtcaactgta
ctgaggtgcccgtagccatccatgcggatcagctcacaccaacttggagggtgtacagc
accggctccaatgtattccagactcgggccggatgccttattggcgccgaacacgtgaa
caatagttacgaatgcgatattccaattggcgccggaatctgtgctagctaccagactc
agacgaactccccaggcagcgccagcagcgttgccagccagtcaatcatcgcttataca
atgtcacttggagccgaaaactccgtggcttactcaaacaacagcatcgccatccccac
aaacttcaccatatccgtgacaactgagattctgccagtgtccatgactaagacgtccg
tagattgcactatgtacatatgcggcgacagcacagaatgttctaatctgctgctgcaa
tatggaagcttctgcactcaactgaacagagcgctcacaggcatcgccgtggagcagga
taagaatacccaggaggtgttcgcccaagttaagcagatctacaagaccccacccataa
aggatttcggtggattcaattttagtcagatactcccagacccatctaagccatccaag
aggagctttatcgaggatcttttgtttaacaaagttactctggccgacgccggtttcat
caagcagtacggagattgcctcggcgacatcgctgctcgtgacctcatctgtgcgcaaa
agtttaacggtctgacggtgctgcctcccctccttactgatgaaatgatcgcccagtat
accagcgcactcctcgctggcaccataacatccggttggacattcggcgctggtgcagc
actgcagataccattcgccatgcaaatggcatatcgtttcaacggtatcggtgtcacac
agaatgtcctatatgagaaccagaagctgatcgcaaatcagttcaatagtgccatcgga
aaaatccaggatagccttagcagcacagcctcagcccttggcaaactccaggatgtcgt
gaaccagaatgcccaggctctcaataccctcgtgaagcagctctcatctaatttcggcg
caatttccagtgtcctcaacgacatcctcagccgcctcgacccccccgaggccgaagtg
cagattgacagactgattacaggtcgactccagagcctccagacttacgtgactcagca
gctgataagagccgccgagataagggccagcgctaacctggctgccacaaagatgtctg
agtgcgtgctgggccagtccaagagagtagacttctgtggcaaaggctaccatctgatg
agcttcccacaatccgcacctcacggcgtagtgttcctccacgtgacatatgtaccggc
tcaggagaagaatttcactaccgctcctgctatatgccatgatggaaaggctcacttcc
cccgggagggggtgttcgtgtccaacggcacccattggtttgtgactcagcggaatttc
tacgaaccccagatcataaccactgacaacacatttgtgtccggaaattgtgacgtggt
cattggaatagtgaacaacactgtttatgatccactgcagccagaacttgacagcttta
aggaggagctcgacaagtacttcaagaatcatacgtcaccagatgtggacctcggagat
attagcggtatcaatgccagtgttgtcaatattcagaaggaaatagaccgccttaatga
ggtcgccaaaaatctgaacgagagcctcatcgatcttcaggagctgggcaaatatgagc
agtacatcaagtggccttggtatatttggcttggcttcatcgccggcctgatcgccata
gtaatggtcacaattatgctctctttatggatgtgctccaatggatcgttacaatgcag
aatttgcatttaa
B CoV S (GSAS-2P) AA
SEQ ID NO: 4
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNEKNLREFVFKNIDGYFKIYSKHTPINLVRDLPQGESALE
PLVDLPIGINITRFQTLLALHRSYLTPGDSSSGWTAGAAAYYVGYLQPRTELLKYNENG
TITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELLH
APATVCGPKKSTNLVKNKCVNFNFNGLTGTGVLTESNKKELPFQQFGRDIADTTDAVRD
PQTLEILDITPCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPSK
RSFIEDLLENKVTLADAGFIKQYGDCLGDIAARDLICAQKENGLTVLPPLLTDEMIAQY
TSALLAGTITSGWTFGAGAALQIPFAMQMAYRENGIGVTQNVLYENQKLIANQFNSAIG
KIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQRNE
YEPQIITTDNTFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIWLGFIAGLIAI
VMVTIMLSLWMCSNGSLQCRICI
C.37 CoV S (GSAS-2P) DNA
SEQ ID NO: 5
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacGTGATCaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccacAACtcctctagcggctggactgccggcgctgccgcttattacgttggttatcttc
agccacgcacgttcctgctgaagtataacgagaatggtactattaccgatgccgtggat
tgtgcccttgaccccctgtccgaaactaagtgcacactcaagtcattcactgtggaaaa
aggaatctaccagacaagcaattttcgggtccagcctactgagagcattgtgcgctttc
ctaacatcacaaatctttgccccttcggagaggttttcaatgctacacggtttgcctcc
gtgtatgcctggaaccgcaagagaatttccaattgcgtggccgattactccgtgctcta
caatagtgcaagctttagcacctttaagtgctatggcgtatcccctactaagcttaacg
acttgtgtttcacaaacgtgtatgccgactcctttgtgatacggggcgacgaagttaga
cagatagcaccaggacagacgggaaagatagctgactacaactataagcttcctgatga
cttcactggctgcgttatcgcgtggaattctaacaacctggactcaaaagtcggcggca
actataactatCAGtatcggctgttccgcaagagtaaccttaagccctttgagagagat
ataagcactgaaatctaccaggctggcagtacgccctgtaatggcgtggaaggctttaa
ttgttatAGCccactgcaatcctatggttttcagccaaccaatggcgtgggctaccaac
cataccgcgtcgtggtgctctcctttgaactgctccacgctcccgcgactgtctgcggc
cccaagaagtccacgaaccttgtgaagaataagtgcgttaattttaatttcaacggcct
cactggaacaggagtgctcactgagagtaacaagaagttcctgccatttcaacaatttg
gcagagacatagccgatactactgacgccgttagggacccccagaccctcgagattctc
gatataacgccctgctccttcggtggagtttccgtgatcacgccaggcaccaataccag
taaccaggtcgccgtgctgtatcagGGCgtcaactgtactgaggtgcccgtagccatcc
atgcggatcagctcacaccaacttggagggtgtacagcaccggctccaatgtattccag
actcgggccggatgccttattggcgccgaacacgtgaacaatagttacgaatgcgatat
tccaattggcgccggaatctgtgctagctaccagactcagacgaactccccaggcagcg
ccagcagcgttgccagccagtcaatcatcgcttatacaatgtcacttggagccgaaaac
tccgtggcttactcaaacaacagcatcgccatccccacaaacttcaccatatccgtgac
aactgagattctgccagtgtccatgactaagacgtccgtagattgcactatgtacatat
gcggcgacagcacagaatgttctaatctgctgctgcaatatggaagcttctgcactcaa
ctgaacagagcgctcacaggcatcgccgtggagcaggataagaatacccaggaggtgtt
cgcccaagttaagcagatctacaagaccccacccataaaggatttcggtggattcaatt
ttagtcagatactcccagacccatctaagccatccaagaggagctttatcgaggatctt
ttgtttaacaaagttactctggccgacgccggtttcatcaagcagtacggagattgcct
cggcgacatcgctgctcgtgacctcatctgtgcgcaaaagtttaacggtctgAACgtgc
tgcctcccctccttactgatgaaatgatcgcccagtataccagcgcactcctcgctggc
accataacatccggttggacattcggcgctggtgcagcactgcagataccattcgccat
gcaaatggcatatcgtttcaacggtatcggtgtcacacagaatgtcctatatgagaacc
agaagctgatcgcaaatcagttcaatagtgccatcggaaaaatccaggatagccttagc
agcacagcctcagcccttggcaaactccaggatgtcgtgaaccagaatgcccaggctct
caataccctcgtgaagcagctctcatctaatttcggcgcaatttccagtgtcctcaacg
acatcctcagccgcctcgacccccccgaggccgaagtgcagattgacagactgattaca
ggtcgactccagagcctccagacttacgtgactcagcagctgataagagccgccgagat
aagggccagcgctaacctggctgccacaaagatgtctgagtgcgtgctgggccagtcca
agagagtagacttctgtggcaaaggctaccatctgatgagcttcccacaatccgcacct
cacggcgtagtgttcctccacgtgacatatgtaccggctcaggagaagaatttcactac
cgctcctgctatatgccatgatggaaaggctcacttcccccgggagggggtgttcgtgt
ccaacggcacccattggtttgtgactcagcggaatttctacgaaccccagatcataacc
actgacaacacatttgtgtccggaaattgtgacgtggtcattggaatagtgaacaacac
tgtttatgatccactgcagccagaacttgacagctttaaggaggagctcgacaagtact
tcaagaatcatacgtcaccagatgtggacctcggagatattagcggtatcaatgccagt
gttgtcaatattcagaaggaaatagaccgccttaatgaggtcgccaaaaatctgaacga
gagcctcatcgatcttcaggagctgggcaaatatgagcagtacatcaagtggccttggt
atatttggcttggcttcatcgccggcctgatcgccatagtaatggtcacaattatgctc
tctttatggatgtgctccaatggatcgttacaatgcagaatttgcatttaa
C.37 CoV S (GSAS-2P) AA
SEQ ID NO: 6
MAKNVAIFGLLFSLLVLVPSQIFASQCVNLTTRTQLPPAYTNSFTRGVYYPDKVERSSV
LHSTQDLFLPFFSNVTWFHAIHVSGTNVIKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHNSSSGWTAGAAAYYVGYLQPRTFLLKYNENGTITDAVD
CALDPLSETKCTLKSFTVEKGIYQTSNERVQPTESIVRFPNITNLCPFGEVENATRFAS
VYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYQYRLERKSNLKPFERD
ISTEIYQAGSTPCNGVEGENCYSPLQSYGFQPTNGVGYQPYRVVVLSFELLHAPATVCG
PKKSTNLVKNKCVNFNFNGLTGTGVLTESNKKELPFQQFGRDIADTTDAVRDPQTLEIL
DITPCSFGGVSVITPGTNTSNQVAVLYQGVNCTEVPVAIHADQLTPTWRVYSTGSNVFQ
TRAGCLIGAEHVNNSYECDIPIGAGICASYQTQTNSPGSASSVASQSIIAYTMSLGAEN
SVAYSNNSIAIPTNFTISVTTEILPVSMTKTSVDCTMYICGDSTECSNLLLQYGSFCTQ
LNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLNVLPPLLTDEMIAQYTSALLAG
TITSGWTFGAGAALQIPFAMQMAYRENGIGVTQNVLYENQKLIANQFNSAIGKIQDSLS
STASALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGLIAIVMVTIML
SLWMCSNGSLQCRICI
B CoV S (GSAS-2P+del246-252) DNA
SEQ ID NO: 7
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccacgactcctctagcggctggactgccggcgctgccgcttattacgttggttatcttc
agccacgcacgttcctgctgaagtataacgagaatggtactattaccgatgccgtggat
tgtgcccttgaccccctgtccgaaactaagtgcacactcaagtcattcactgtggaaaa
aggaatctaccagacaagcaattttcgggtccagcctactgagagcattgtgcgctttc
ctaacatcacaaatctttgccccttcggagaggttttcaatgctacacggtttgcctcc
gtgtatgcctggaaccgcaagagaatttccaattgcgtggccgattactccgtgctcta
caatagtgcaagctttagcacctttaagtgctatggcgtatcccctactaagcttaacg
acttgtgtttcacaaacgtgtatgccgactcctttgtgatacggggcgacgaagttaga
cagatagcaccaggacagacgggaaagatagctgactacaactataagcttcctgatga
cttcactggctgcgttatcgcgtggaattctaacaacctggactcaaaagtcggcggca
actataactatctctatcggctgttccgcaagagtaaccttaagccctttgagagagat
ataagcactgaaatctaccaggctggcagtacgccctgtaatggcgtggaaggctttaa
ttgttattttccactgcaatcctatggttttcagccaaccaatggcgtgggctaccaac
cataccgcgtcgtggtgctctcctttgaactgctccacgctcccgcgactgtctgcggc
cccaagaagtccacgaaccttgtgaagaataagtgcgttaattttaatttcaacggcct
cactggaacaggagtgctcactgagagtaacaagaagttcctgccatttcaacaatttg
gcagagacatagccgatactactgacgccgttagggacccccagaccctcgagattctc
gatataacgccctgctccttcggtggagtttccgtgatcacgccaggcaccaataccag
taaccaggtcgccgtgctgtatcaggatgtcaactgtactgaggtgcccgtagccatcc
atgcggatcagctcacaccaacttggagggtgtacagcaccggctccaatgtattccag
actcgggccggatgccttattggcgccgaacacgtgaacaatagttacgaatgcgatat
tccaattggcgccggaatctgtgctagctaccagactcagacgaactccccaggcagcg
ccagcagcgttgccagccagtcaatcatcgcttatacaatgtcacttggagccgaaaac
tccgtggcttactcaaacaacagcatcgccatccccacaaacttcaccatatccgtgac
aactgagattctgccagtgtccatgactaagacgtccgtagattgcactatgtacatat
gcggcgacagcacagaatgttctaatctgctgctgcaatatggaagcttctgcactcaa
ctgaacagagcgctcacaggcatcgccgtggagcaggataagaatacccaggaggtgtt
cgcccaagttaagcagatctacaagaccccacccataaaggatttcggtggattcaatt
ttagtcagatactcccagacccatctaagccatccaagaggagctttatcgaggatctt
ttgtttaacaaagttactctggccgacgccggtttcatcaagcagtacggagattgcct
cggcgacatcgctgctcgtgacctcatctgtgcgcaaaagtttaacggtctgacggtgc
tgcctcccctccttactgatgaaatgatcgcccagtataccagcgcactcctcgctggc
accataacatccggttggacattcggcgctggtgcagcactgcagataccattcgccat
gcaaatggcatatcgtttcaacggtatcggtgtcacacagaatgtcctatatgagaacc
agaagctgatcgcaaatcagttcaatagtgccatcggaaaaatccaggatagccttagc
agcacagcctcagcccttggcaaactccaggatgtcgtgaaccagaatgcccaggctct
caataccctcgtgaagcagctctcatctaatttcggcgcaatttccagtgtcctcaacg
acatcctcagccgcctcgacccccccgaggccgaagtgcagattgacagactgattaca
ggtcgactccagagcctccagacttacgtgactcagcagctgataagagccgccgagat
aagggccagcgctaacctggctgccacaaagatgtctgagtgcgtgctgggccagtcca
agagagtagacttctgtggcaaaggctaccatctgatgagcttcccacaatccgcacct
cacggcgtagtgttcctccacgtgacatatgtaccggctcaggagaagaatttcactac
cgctcctgctatatgccatgatggaaaggctcacttcccccggggggggtgttcgtgt
ccaacggcacccattggtttgtgactcagcggaatttctacgaaccccagatcataacc
actgacaacacatttgtgtccggaaattgtgacgtggtcattggaatagtgaacaacac
tgtttatgatccactgcagccagaacttgacagctttaaggaggagctcgacaagtact
tcaagaatcatacgtcaccagatgtggacctcggagatattagcggtatcaatgccagt
gttgtcaatattcagaaggaaatagaccgccttaatgaggtcgccaaaaatctgaacga
gagcctcatcgatcttcaggagctgggcaaatatgagcagtacatcaagtggccttggt
atatttggcttggcttcatcgccggcctgatcgccatagtaatggtcacaattatgctc
tctttatggatgtgctccaatggatcgttacaatgcagaatttgcatttaa
B CoV S (GSAS-2P+del246-252) AA
SEQ ID NO: 8
MAKNVAIFGLLFSLLVLVPSQIFASQCVNLTTRTQLPPAYTNSFTRGVYYPDKVFRSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHDSSSGWTAGAAAYYVGYLQPRTFLLKYNENGTITDAVD
CALDPLSETKCTLKSFTVEKGIYQTSNERVQPTESIVRFPNITNLCPFGEVENATRFAS
VYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLERKSNLKPFERD
ISTEIYQAGSTPCNGVEGENCYFPLQSYGFQPTNGVGYQPYRVVVLSFELLHAPATVCG
PKKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPGSASSVASQSIIAYTMSLGAEN
SVAYSNNSIAIPTNFTISVTTEILPVSMTKTSVDCTMYICGDSTECSNLLLQYGSFCTQ
LNRALTGIAVEQDKNTQEVFAQVKQIYKTPPIKDFGGFNFSQILPDPSKPSKRSFIEDL
LENKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLS
STASALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGLIAIVMVTIML
SLWMCSNGSLQCRICI*
B CoV S (GSAS-2P+D252N) DNA
SEQ ID NO: 9
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatcctatctgacacccggaAACtcctctagcggctggactgccggcgctgccg
cttattacgttggttatcttcagccacgcacgttcctgctgaagtataacgagaatggt
actattaccgatgccgtggattgtgcccttgaccccctgtccgaaactaagtgcacact
caagtcattcactgtggaaaaaggaatctaccagacaagcaattttcgggtccagccta
ctgagagcattgtgcgctttcctaacatcacaaatctttgccccttcggagaggttttc
aatgctacacggtttgcctccgtgtatgcctggaaccgcaagagaatttccaattgcgt
ggccgattactccgtgctctacaatagtgcaagctttagcacctttaagtgctatggcg
tatcccctactaagcttaacgacttgtgtttcacaaacgtgtatgccgactcctttgtg
atacggggcgacgaagttagacagatagcaccaggacagacgggaaagatagctgacta
caactataagcttcctgatgacttcactggctgcgttatcgcgtggaattctaacaacc
tggactcaaaagtcggcggcaactataactatctctatcggctgttccgcaagagtaac
cttaagccctttgagagagatataagcactgaaatctaccaggctggcagtacgccctg
taatggcgtggaaggctttaattgttattttccactgcaatcctatggttttcagccaa
ccaatggcgtgggctaccaaccataccgcgtcgtggtgctctcctttgaactgctccac
gctcccgcgactgtctgcggccccaagaagtccacgaaccttgtgaagaataagtgcgt
taattttaatttcaacggcctcactggaacaggagtgctcactgagagtaacaagaagt
tcctgccatttcaacaatttggcagagacatagccgatactactgacgccgttagggac
ccccagaccctcgagattctcgatataacgccctgctccttcggtggagtttccgtgat
cacgccaggcaccaataccagtaaccaggtcgccgtgctgtatcaggatgtcaactgta
ctgaggtgcccgtagccatccatgcggatcagctcacaccaacttggagggtgtacagc
accggctccaatgtattccagactcgggccggatgccttattggcgccgaacacgtgaa
caatagttacgaatgcgatattccaattggcgccggaatctgtgctagctaccagactc
agacgaactccccaggcagcgccagcagcgttgccagccagtcaatcatcgcttataca
atgtcacttggagccgaaaactccgtggcttactcaaacaacagcatcgccatccccac
aaacttcaccatatccgtgacaactgagattctgccagtgtccatgactaagacgtccg
tagattgcactatgtacatatgcggcgacagcacagaatgttctaatctgctgctgcaa
tatggaagcttctgcactcaactgaacagagcgctcacaggcatcgccgtggagcagga
taagaatacccaggaggtgttcgcccaagttaagcagatctacaagaccccacccataa
aggatttcggtggattcaattttagtcagatactcccagacccatctaagccatccaag
aggagctttatcgaggatcttttgtttaacaaagttactctggccgacgccggtttcat
caagcagtacggagattgcctcggcgacatcgctgctcgtgacctcatctgtgcgcaaa
agtttaacggtctgacggtgctgcctcccctccttactgatgaaatgatcgcccagtat
accagcgcactcctcgctggcaccataacatccggttggacattcggcgctggtgcagc
actgcagataccattcgccatgcaaatggcatatcgtttcaacggtatcggtgtcacac
agaatgtcctatatgagaaccagaagctgatcgcaaatcagttcaatagtgccatcgga
aaaatccaggatagccttagcagcacagcctcagcccttggcaaactccaggatgtcgt
gaaccagaatgcccaggctctcaataccctcgtgaagcagctctcatctaatttcggcg
caatttccagtgtcctcaacgacatcctcagccgcctcgacccccccgaggccgaagtg
cagattgacagactgattacaggtcgactccagagcctccagacttacgtgactcagca
gctgataagagccgccgagataagggccagcgctaacctggctgccacaaagatgtctg
agtgcgtgctgggccagtccaagagagtagacttctgtggcaaaggctaccatctgatg
agcttcccacaatccgcacctcacggcgtagtgttcctccacgtgacatatgtaccggc
tcaggagaagaatttcactaccgctcctgctatatgccatgatggaaaggctcacttcc
cccgggagggggtgttcgtgtccaacggcacccattggtttgtgactcagcggaatttc
tacgaaccccagatcataaccactgacaacacatttgtgtccggaaattgtgacgtggt
cattggaatagtgaacaacactgtttatgatccactgcagccagaacttgacagcttta
aggaggagctcgacaagtacttcaagaatcatacgtcaccagatgtggacctcggagat
attagcggtatcaatgccagtgttgtcaatattcagaaggaaatagaccgccttaatga
ggtcgccaaaaatctgaacgagagcctcatcgatcttcaggagctgggcaaatatgagc
agtacatcaagtggccttggtatatttggcttggcttcatcgccggcctgatcgccata
gtaatggtcacaattatgctctctttatggatgtgctccaatggatcgttacaatgcag
aatttgcatttaa
B CoV S (GSAS-2P+D252N) AA
SEQ ID NO: 10
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRSYLTPGNSSSGWTAGAAAYYVGYLQPRTELLKYNENG
TITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELLH
APATVCGPKKSTNLVKNKCVNFNFNGLTGTGVLTESNKKELPFQQFGRDIADTTDAVRD
PQTLEILDITPCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPSK
RSFIEDLLENKVTLADAGFIKQYGDCLGDIAARDLICAQKENGLTVLPPLLTDEMIAQY
TSALLAGTITSGWTFGAGAALQIPFAMQMAYRENGIGVTQNVLYENQKLIANQFNSAIG
KIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQRNE
YEPQIITTDNTFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIWLGFIAGLIAI
VMVTIMLSLWMCSNGSLQCRICI
B CoV S (GSAS-2P+del246-252+D253N) DNA
SEQ ID NO: 11
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccacAACtcctctagcggctggactgccggcgctgccgcttattacgttggttatcttc
agccacgcacgttcctgctgaagtataacgagaatggtactattaccgatgccgtggat
tgtgcccttgaccccctgtccgaaactaagtgcacactcaagtcattcactgtggaaaa
aggaatctaccagacaagcaattttcgggtccagcctactgagagcattgtgcgctttc
ctaacatcacaaatctttgccccttcggagaggttttcaatgctacacggtttgcctcc
gtgtatgcctggaaccgcaagagaatttccaattgcgtggccgattactccgtgctcta
caatagtgcaagctttagcacctttaagtgctatggcgtatcccctactaagcttaacg
acttgtgtttcacaaacgtgtatgccgactcctttgtgatacggggcgacgaagttaga
cagatagcaccaggacagacgggaaagatagctgactacaactataagcttcctgatga
cttcactggctgcgttatcgcgtggaattctaacaacctggactcaaaagtcggcggca
actataactatctctatcggctgttccgcaagagtaaccttaagccctttgagagagat
ataagcactgaaatctaccaggctggcagtacgccctgtaatggcgtggaaggctttaa
ttgttattttccactgcaatcctatggttttcagccaaccaatggcgtgggctaccaac
cataccgcgtcgtggtgctctcctttgaactgctccacgctcccgcgactgtctgcggc
cccaagaagtccacgaaccttgtgaagaataagtgcgttaattttaatttcaacggcct
cactggaacaggagtgctcactgagagtaacaagaagttcctgccatttcaacaatttg
gcagagacatagccgatactactgacgccgttagggacccccagaccctcgagattctc
gatataacgccctgctccttcggtggagtttccgtgatcacgccaggcaccaataccag
taaccaggtcgccgtgctgtatcaggatgtcaactgtactgaggtgcccgtagccatcc
atgcggatcagctcacaccaacttggagggtgtacagcaccggctccaatgtattccag
actcgggccggatgccttattggcgccgaacacgtgaacaatagttacgaatgcgatat
tccaattggcgccggaatctgtgctagctaccagactcagacgaactccccaggcagcg
ccagcagcgttgccagccagtcaatcatcgcttatacaatgtcacttggagccgaaaac
tccgtggcttactcaaacaacagcatcgccatccccacaaacttcaccatatccgtgac
aactgagattctgccagtgtccatgactaagacgtccgtagattgcactatgtacatat
gcggcgacagcacagaatgttctaatctgctgctgcaatatggaagcttctgcactcaa
ctgaacagagcgctcacaggcatcgccgtggagcaggataagaatacccaggaggtgtt
cgcccaagttaagcagatctacaagaccccacccataaaggatttcggtggattcaatt
ttagtcagatactcccagacccatctaagccatccaagaggagctttatcgaggatctt
ttgtttaacaaagttactctggccgacgccggtttcatcaagcagtacggagattgcct
cggcgacatcgctgctcgtgacctcatctgtgcgcaaaagtttaacggtctgacggtgc
tgcctcccctccttactgatgaaatgatcgcccagtataccagcgcactcctcgctggc
accataacatccggttggacattcggcgctggtgcagcactgcagataccattcgccat
gcaaatggcatatcgtttcaacggtatcggtgtcacacagaatgtcctatatgagaacc
agaagctgatcgcaaatcagttcaatagtgccatcggaaaaatccaggatagccttagc
agcacagcctcagcccttggcaaactccaggatgtcgtgaaccagaatgcccaggctct
caataccctcgtgaagcagctctcatctaatttcggcgcaatttccagtgtcctcaacg
acatcctcagccgcctcgacccccccgaggccgaagtgcagattgacagactgattaca
ggtcgactccagagcctccagacttacgtgactcagcagctgataagagccgccgagat
aagggccagcgctaacctggctgccacaaagatgtctgagtgcgtgctgggccagtcca
agagagtagacttctgtggcaaaggctaccatctgatgagcttcccacaatccgcacct
cacggcgtagtgttcctccacgtgacatatgtaccggctcaggagaagaatttcactac
cgctcctgctatatgccatgatggaaaggctcacttcccccgggagggggtgttcgtgt
ccaacggcacccattggtttgtgactcagcggaatttctacgaaccccagatcataacc
actgacaacacatttgtgtccggaaattgtgacgtggtcattggaatagtgaacaacac
tgtttatgatccactgcagccagaacttgacagctttaaggaggagctcgacaagtact
tcaagaatcatacgtcaccagatgtggacctcggagatattagcggtatcaatgccagt
gttgtcaatattcagaaggaaatagaccgccttaatgaggtcgccaaaaatctgaacga
gagcctcatcgatcttcaggagctgggcaaatatgagcagtacatcaagtggccttggt
atatttggcttggcttcatcgccggcctgatcgccatagtaatggtcacaattatgctc
tctttatggatgtgctccaatggatcgttacaatgcagaatttgcatttaa
B CoV S (GSAS-2P+del246-252+D253N) AA
SEQ ID NO: 12
MAKNVAIFGLLFSLLVLVPSQIFASQCVNLTTRTQLPPAYTNSFTRGVYYPDKVERSSV
LHSTQDLFLPFFSNVTWFHAIHVSGINGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHNSSSGWTAGAAAYYVGYLQPRTELLKYNENGTITDAVD
CALDPLSETKCTLKSFTVEKGIYQTSNERVQPTESIVRFPNITNLCPFGEVENATRFAS
VYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPFERD
ISTEIYQAGSTPCNGVEGENCYFPLQSYGFQPTNGVGYQPYRVVVLSFELLHAPATVCG
PKKSTNLVKNKCVNFNFNGLTGTGVLTESNKKELPFQQFGRDIADTTDAVRDPQTLEIL
DITPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPGSASSVASQSIIAYTMSLGAEN
SVAYSNNSIAIPTNFTISVTTEILPVSMTKTSVDCTMYICGDSTECSNLLLQYGSFCTQ
LNRALTGIAVEQDKNTQEVFAQVKQIYKTPPIKDFGGFNFSQILPDPSKPSKRSFIEDL
LENKVTLADAGFIKQYGDCLGDIAARDLICAQKENGLTVLPPLLTDEMIAQYTSALLAG
TITSGWTFGAGAALQIPFAMQMAYRENGIGVTQNVLYENQKLIANQFNSAIGKIQDSLS
STASALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGLIAIVMVTIML
SLWMCSNGSLQCRICI
B.1.617.2 COV S (GSAS-2P) DNA
SEQ ID NO: 13
atggcgaaaaacgttgcgattttcggcttattgttttctcttcttgtgttggttccttc
tcagatcttcgcgTCCCAATGCgtgaatcttAGAacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgGACgtgtactatc
ataagaacaacaagtcttggatggaatctGGCgtctatagcagcgccaacaactgcacc
tttgaatacgtgtcccagcccttccttatggacctggagggaaagcagggaaactttaa
gaatctgagagagttcgtgtttaaaaatatcgacggctattttaagatctattctaagc
acacgcctattaatctcgtgcgcgatcttccacaaggcttcagcgccctggaaccactc
gtggacctcccaattggtatcaacatcactagatttcagactctgcttgccctccaccg
atcctatctgacacccggagactcctctagcggctggactgccggcgctgccgcttatt
acgttggttatcttcagccacgcacgttcctgctgaagtataacgagaatggtactatt
accgatgccgtggattgtgcccttgaccccctgtccgaaactaagtgcacactcaagtc
attcactgtggaaaaaggaatctaccagacaagcaattttcgggtccagcctactgaga
gcattgtgcgctttcctaacatcacaaatctttgccccttcggagaggttttcaatgct
acacggtttgcctccgtgtatgcctggaaccgcaagagaatttccaattgcgtggccga
ttactccgtgctctacaatagtgcaagctttagcacctttaagtgctatggcgtatccc
ctactaagcttaacgacttgtgtttcacaaacgtgtatgccgactcctttgtgatacgg
ggcgacgaagttagacagatagcaccaggacagacgggaaagatagctgactacaacta
taagcttcctgatgacttcactggctgcgttatcgcgtggaattctaacaacctggact
caaaagtcggcggcaactataactatAGAtatcggctgttccgcaagagtaaccttaag
ccctttgagagagatataagcactgaaatctaccaggctggcagtAAGccctgtaatgg
cgtgGAGggctttaattgttattttccactgcaatcctatggttttcagccaaccaatg
gcgtgggctaccaaccataccgcgtcgtggtgctctcctttgaactgctccacgctccc
gcgactgtctgcggccccaagaagtccacgaaccttgtgaagaataagtgcgttaattt
taatttcaacggcctcactggaacaggagtgctcactgagagtaacaagaagttcctgc
catttcaacaatttggcagagacatagccgatactactgacgccgttagggacccccag
accctcgagattctcgatataacgccctgctccttcggtggagtttccgtgatcacgcc
aggcaccaataccagtaaccaggtcgccgtgctgtatcagGGCgtcaactgtactgagg
tgcccgtagccatccatgcggatcagctcacaccaacttggagggtgtacagcaccggc
tccaatgtattccagactcgggccggatgccttattggcgccgaacacgtgaacaatag
ttacgaatgcgatattccaattggcgccggaatctgtgctagctaccagactcagacga
actccAGAggcagcgccagcagcgttgccagccagtcaatcatcgcttatacaatgtca
cttggagccgaaaactccgtggcttactcaaacaacagcatcgccatccccacaaactt
caccatatccgtgacaactgagattctgccagtgtccatgactaagacgtccgtagatt
gcactatgtacatatgcggcgacagcacagaatgttctaatctgctgctgcaatatgga
agcttctgcactcaactgaacagagcgctcacaggcatcgccgtggagcaggataagaa
tacccaggaggtgttcgcccaagttaagcagatctacaagaccccacccataaaggatt
tcggtggattcaattttagtcagatactcccagacccatctaagccatccaagaggagc
tttatcgaggatcttttgtttaacaaagttactctggccgacgccggtttcatcaagca
gtacggagattgcctcggcgacatcgctgctcgtgacctcatctgtgcgcaaaagttta
acggtctgacggtgctgcctcccctccttactgatgaaatgatcgcccagtataccagc
gcactcctcgctggcaccataacatccggttggacattcggcgctggtgcagcactgca
gataccattcgccatgcaaatggcatatcgtttcaacggtatcggtgtcacacagaatg
tcctatatgagaaccagaagctgatcgcaaatcagttcaatagtgccatcggaaaaatc
caggatagccttagcagcacagcctcagcccttggcaaactccagAACgtcgtgaacca
gaatgcccaggctctcaataccctcgtgaagcagctctcatctaatttcggcgcaattt
ccagtgtcctcaacgacatcctcagccgcctcgacccccccgaggccgaagtgcagatt
gacagactgattacaggtcgactccagagcctccagacttacgtgactcagcagctgat
aagagccgccgagataagggccagcgctaacctggctgccacaaagatgtctgagtgcg
tgctgggccagtccaagagagtagacttctgtggcaaaggctaccatctgatgagcttc
ccacaatccgcacctcacggcgtagtgttcctccacgtgacatatgtaccggctcagga
gaagaatttcactaccgctcctgctatatgccatgatggaaaggctcacttcccccggg
agggggtgttcgtgtccaacggcacccattggtttgtgactcagcggaatttctacgaa
ccccagatcataaccactgacaacacatttgtgtccggaaattgtgacgtggtcattgg
aatagtgaacaacactgtttatgatccactgcagccagaacttgacagctttaaggagg
agctcgacaagtacttcaagaatcatacgtcaccagatgtggacctcggagatattagc
ggtatcaatgccagtgttgtcaatattcagaaggaaatagaccgccttaatgaggtcgc
caaaaatctgaacgagagcctcatcgatcttcaggagctgggcaaatatgagcagtaca
tcaagtggccttggtatatttggcttggcttcatcgccggcctgatcgccatagtaatg
gtcacaattatgctctctttatggatgtgctccaatggatcgttacaatgcagaatttg
catttaa
B.1.617.2 CoV S (GSAS-2P) AA
SEQ ID NO: 14
MAKNVAIFGLLFSLLVLVPSQIFASQCVNLRTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLDVYYHKNNKSWMESGVYSSANNCT
FEYVSQPFLMDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTELLKYNENGTI
TDAVDCALDPLSETKCTLKSFTVEKGIYQTSNERVQPTESIVRFPNITNLCPFGEVENA
TRFASVYAWNRKRISNCVADYSVLYNSASFSTFKCYGVSPTKLNDLCFTNVYADSEVIR
GDEVRQIAPGQTGKIADYNYKLPDDFTGCVIAWNSNNLDSKVGGNYNYRYRLFRKSNLK
PFERDISTEIYQAGSKPCNGVEGENCYFPLQSYGFQPTNGVGYQPYRVVVLSFELLHAP
ATVCGPKKSTNLVKNKCVNFNFNGLTGTGVLTESNKKELPFQQFGRDIADTTDAVRDPQ
TLEILDITPCSFGGVSVITPGTNTSNQVAVLYQGVNCTEVPVAIHADQLTPTWRVYSTG
SNVFQTRAGCLIGAEHVNNSYECDIPIGAGICASYQTQTNSRGSASSVASQSIIAYTMS
LGAENSVAYSNNSIAIPTNFTISVTTEILPVSMTKTSVDCTMYICGDSTECSNLLLQYG
SFCTQLNRALTGIAVEQDKNTQEVFAQVKQIYKTPPIKDFGGFNFSQILPDPSKPSKRS
FIEDLLENKVTLADAGFIKQYGDCLGDIAARDLICAQKENGLTVLPPLLTDEMIAQYTS
ALLAGTITSGWTFGAGAALQIPFAMQMAYRENGIGVTQNVLYENQKLIANQFNSAIGKI
QDSLSSTASALGKLQNVVNQNAQALNTLVKQLSSNFGAISSVLNDILSRLDPPEAEVQI
DRLITGRLQSLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIVM
VTIMLSLWMCSNGSLQCRICI
B.1.617.2 CoV S (GSAS-2P+D253N) DNA
SEQ ID NO: 15
atggcgaaaaacgttgcgattttcggcttattgttttctcttcttgtgttggttccttc
tcagatcttcgcgTCCCAATGCgtgaatcttAGAacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgGACgtgtactatc
ataagaacaacaagtcttggatggaatctGGCgtctatagcagcgccaacaactgcacc
tttgaatacgtgtcccagcccttccttatggacctggagggaaagcagggaaactttaa
gaatctgagagagttcgtgtttaaaaatatcgacggctattttaagatctattctaagc
acacgcctattaatctcgtgcgcgatcttccacaaggcttcagcgccctggaaccactc
gtggacctcccaattggtatcaacatcactagatttcagactctgcttgccctccaccg
atcctatctgacacccggaAACtcctctagcggctggactgccggcgctgccgcttatt
acgttggttatcttcagccacgcacgttcctgctgaagtataacgagaatggtactatt
accgatgccgtggattgtgcccttgaccccctgtccgaaactaagtgcacactcaagtc
attcactgtggaaaaaggaatctaccagacaagcaattttcgggtccagcctactgaga
gcattgtgcgctttcctaacatcacaaatctttgccccttcggagaggttttcaatgct
acacggtttgcctccgtgtatgcctggaaccgcaagagaatttccaattgcgtggccga
ttactccgtgctctacaatagtgcaagctttagcacctttaagtgctatggcgtatccc
ctactaagcttaacgacttgtgtttcacaaacgtgtatgccgactcctttgtgatacgg
ggcgacgaagttagacagatagcaccaggacagacgggaaagatagctgactacaacta
taagcttcctgatgacttcactggctgcgttatcgcgtggaattctaacaacctggact
caaaagtcggcggcaactataactatAGAtatcggctgttccgcaagagtaaccttaag
ccctttgagagagatataagcactgaaatctaccaggctggcagtAAGccctgtaatgg
cgtgGAGggctttaattgttattttccactgcaatcctatggttttcagccaaccaatg
gcgtgggctaccaaccataccgcgtcgtggtgctctcctttgaactgctccacgctccc
gcgactgtctgcggccccaagaagtccacgaaccttgtgaagaataagtgcgttaattt
taatttcaacggcctcactggaacaggagtgctcactgagagtaacaagaagttcctgc
catttcaacaatttggcagagacatagccgatactactgacgccgttagggacccccag
accctcgagattctcgatataacgccctgctccttcggtggagtttccgtgatcacgcc
aggcaccaataccagtaaccaggtcgccgtgctgtatcagGGCgtcaactgtactgagg
tgcccgtagccatccatgcggatcagctcacaccaacttggagggtgtacagcaccggc
tccaatgtattccagactcgggccggatgccttattggcgccgaacacgtgaacaatag
ttacgaatgcgatattccaattggcgccggaatctgtgctagctaccagactcagacga
actccAGAggcagcgccagcagcgttgccagccagtcaatcatcgcttatacaatgtca
cttggagccgaaaactccgtggcttactcaaacaacagcatcgccatccccacaaactt
caccatatccgtgacaactgagattctgccagtgtccatgactaagacgtccgtagatt
gcactatgtacatatgcggcgacagcacagaatgttctaatctgctgctgcaatatgga
agcttctgcactcaactgaacagagcgctcacaggcatcgccgtggagcaggataagaa
tacccaggaggtgttcgcccaagttaagcagatctacaagaccccacccataaaggatt
tcggtggattcaattttagtcagatactcccagacccatctaagccatccaagaggagc
tttatcgaggatcttttgtttaacaaagttactctggccgacgccggtttcatcaagca
gtacggagattgcctcggcgacatcgctgctcgtgacctcatctgtgcgcaaaagttta
acggtctgacggtgctgcctcccctccttactgatgaaatgatcgcccagtataccagc
gcactcctcgctggcaccataacatccggttggacattcggcgctggtgcagcactgca
gataccattcgccatgcaaatggcatatcgtttcaacggtatcggtgtcacacagaatg
tcctatatgagaaccagaagctgatcgcaaatcagttcaatagtgccatcggaaaaatc
caggatagccttagcagcacagcctcagcccttggcaaactccagAACgtcgtgaacca
gaatgcccaggctctcaataccctcgtgaagcagctctcatctaatttcggcgcaattt
ccagtgtcctcaacgacatcctcagccgcctcgacccccccgaggccgaagtgcagatt
gacagactgattacaggtcgactccagagcctccagacttacgtgactcagcagctgat
aagagccgccgagataagggccagcgctaacctggctgccacaaagatgtctgagtgcg
tgctgggccagtccaagagagtagacttctgtggcaaaggctaccatctgatgagcttc
ccacaatccgcacctcacggcgtagtgttcctccacgtgacatatgtaccggctcagga
gaagaatttcactaccgctcctgctatatgccatgatggaaaggctcacttcccccggg
agggggtgttcgtgtccaacggcacccattggtttgtgactcagcggaatttctacgaa
ccccagatcataaccactgacaacacatttgtgtccggaaattgtgacgtggtcattgg
aatagtgaacaacactgtttatgatccactgcagccagaacttgacagctttaaggagg
agctcgacaagtacttcaagaatcatacgtcaccagatgtggacctcggagatattagc
ggtatcaatgccagtgttgtcaatattcagaaggaaatagaccgccttaatgaggtcgc
caaaaatctgaacgagagcctcatcgatcttcaggagctgggcaaatatgagcagtaca
tcaagtggccttggtatatttggcttggcttcatcgccggcctgatcgccatagtaatg
gtcacaattatgctctctttatggatgtgctccaatggatcgttacaatgcagaatttg
catttaa
B.1.617.2 CoV S (GSAS-2P+D253N) AA
SEQ ID NO: 16
MAKNVAIFGLLFSLLVLVPSQIFASQCVNLRTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPELDVYYHKNNKSWMESGVYSSANNCT
FEYVSQPFLMDLEGKQGNFKNLREFVFKNIDGYFKIYSKHTPINLVRDLPQGFSALEPL
VDLPIGINITRFQTLLALHRSYLTPGNSSSGWTAGAAAYYVGYLQPRTELLKYNENGTI
TDAVDCALDPLSETKCTLKSFTVEKGIYQTSNERVQPTESIVRFPNITNLCPFGEVENA
TRFASVYAWNRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYRYRLFRKSNLK
PFERDISTEIYQAGSKPCNGVEGENCYFPLQSYGFQPTNGVGYQPYRVVVLSFELLHAP
ATVCGPKKSTNLVKNKCVNFNFNGLTGTGVLTESNKKELPFQQFGRDIADTTDAVRDPQ
TLEILDITPCSFGGVSVITPGTNTSNQVAVLYQGVNCTEVPVAIHADQLTPTWRVYSTG
SNVFQTRAGCLIGAEHVNNSYECDIPIGAGICASYQTQTNSRGSASSVASQSIIAYTMS
LGAENSVAYSNNSIAIPTNFTISVTTEILPVSMTKTSVDCTMYICGDSTECSNLLLQYG
SFCTQLNRALTGIAVEQDKNTQEVFAQVKQIYKTPPIKDFGGFNFSQILPDPSKPSKRS
FIEDLLENKVTLADAGFIKQYGDCLGDIAARDLICAQKENGLTVLPPLLTDEMIAQYTS
ALLAGTITSGWTFGAGAALQIPFAMQMAYRENGIGVTQNVLYENQKLIANQFNSAIGKI
QDSLSSTASALGKLQNVVNQNAQALNTLVKQLSSNFGAISSVLNDILSRLDPPEAEVQI
DRLITGRLQSLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIVM
VTIMLSLWMCSNGSLQCRICI
B.1.1.529 COV S (GSAS-2P) DNA
SEQ ID NO: 17
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatGT
Gatttccggcactaacggtactaagaggttcgataatcctgtgctccctttcaatgacg
gcgtttactttgcaagcATCgagaagagtaacatcatccgaggttggatctttggcact
accctcgattcaaagacgcagagcctcctcattgtgaacaatgccactaacgtggtgat
caaagtttgcgagtttcagttctgcaatgaccctttcTTGGACcataagaacaacaagt
cttggatggaatctgaattccgcgtctatagcagcgccaacaactgcacctttgaatac
gtgtcccagcccttccttatggacctggagggaaagcagggaaactttaagaatctgag
agagttcgtgtttaaaaatatcgacggctattttaagatctattctaagcacacgccta
ttATCgtgcgcGAGCCCGAGgatcttccacaaggcttcagcgccctggaaccactcgtg
gacctcccaattggtatcaacatcactagatttcagactctgcttgccctccaccgatc
ctatctgacacccggagactcctctagcggctggactgccggcgctgccgcttattacg
ttggttatcttcagccacgcacgttcctgctgaagtataacgagaatggtactattacc
gatgccgtggattgtgcccttgaccccctgtccgaaactaagtgcacactcaagtcatt
cactgtggaaaaaggaatctaccagacaagcaattttcgggtccagcctactgagagca
ttgtgcgctttcctaacatcacaaatctttgccccttcGACgaggttttcaatgctaca
cggtttgcctccgtgtatgcctggaaccgcaagagaatttccaattgcgtggccgatta
ctccgtgctctacaatCTGgcaCCCtttTTCacctttaagtgctatggcgtatccccta
ctaagcttaacgacttgtgtttcacaaacgtgtatgccgactcctttgtgatacggggc
gacgaagttagacagatagcaccaggacagacgggaAACatagctgactacaactataa
gcttcctgatgacttcactggctgcgttatcgcgtggaattctaacAAGctggactcaa
aagtcAGCggcaactataactatctctatcggctgttccgcaagagtaaccttaagccc
tttgagagagatataagcactgaaatctaccaggctggcAACAAGccctgtaatggcgt
gGCCggctttaattgttattttccactgAGAtcctatAGCtttAGAccaaccTACggcg
tgggcCACcaaccataccgcgtcgtggtgctctcctttgaactgctccacgctcccgcg
actgtctgcggccccaagaagtccacgaaccttgtgaagaataagtgcgttaattttaa
tttcaacggcctcAAGggaacaggagtgctcactgagagtaacaagaagttcctgccat
ttcaacaatttggcagagacatagccgatactactgacgccgttagggacccccagacc
ctcgagattctcgatataacgccctgctccttcggtggagtttccgtgatcacgccagg
caccaataccagtaaccaggtcgccgtgctgtatcagGGCgtcaactgtactgaggtgc
ccgtagccatccatgcggatcagctcacaccaacttggagggtgtacagcaccggctcc
aatgtattccagactcgggccggatgccttattggcgccgaaTACgtgaacaatagtta
cgaatgcgatattccaattggcgccggaatctgtgctagctaccagactcagacgAAGt
ccCACggcagcgccagcagcgttgccagccagtcaatcatcgcttatacaatgtcactt
ggagccgaaaactccgtggcttactcaaacaacagcatcgccatccccacaaacttcac
catatccgtgacaactgagattctgccagtgtccatgactaagacgtccgtagattgca
ctatgtacatatgcggcgacagcacagaatgttctaatctgctgctgcaatatggaagc
ttctgcactcaactgAAGagagcgctcacaggcatcgccgtggagcaggataagaatac
ccaggaggtgttcgcccaagttaagcagatctacaagaccccacccataaagTACttcg
gtggattcaattttagtcagatactcccagacccatctaagccatccaagaggagcttt
atcgaggatcttttgtttaacaaagttactctggccgacgccggtttcatcaagcagta
cggagattgcctcggcgacatcgctgctcgtgacctcatctgtgcgcaaaagtttAAGg
gtctgacggtgctgcctcccctccttactgatgaaatgatcgcccagtataccagcgca
ctcctcgctggcaccataacatccggttggacattcggcgctggtgcagcactgcagat
accattcgccatgcaaatggcatatcgtttcaacggtatcggtgtcacacagaatgtcc
tatatgagaaccagaagctgatcgcaaatcagttcaatagtgccatcggaaaaatccag
gatagccttagcagcacagcctcagcccttggcaaactccaggatgtcgtgaacCACaa
tgcccaggctctcaataccctcgtgaagcagctctcatctAAGttcggcgcaatttcca
gtgtcctcaacgacatcTTCagccgcctcgacccccccgaggccgaagtgcagattgac
agactgattacaggtcgactccagagcctccagacttacgtgactcagcagctgataag
agccgccgagataagggccagcgctaacctggctgccacaaagatgtctgagtgcgtgc
tgggccagtccaagagagtagacttctgtggcaaaggctaccatctgatgagcttccca
caatccgcacctcacggcgtagtgttcctccacgtgacatatgtaccggctcaggagaa
gaatttcactaccgctcctgctatatgccatgatggaaaggctcacttcccccgggagg
gggtgttcgtgtccaacggcacccattggtttgtgactcagcggaatttctacgaaccc
cagatcataaccactgacaacacatttgtgtccggaaattgtgacgtggtcattggaat
agtgaacaacactgtttatgatccactgcagccagaacttgacagctttaaggaggagc
tcgacaagtacttcaagaatcatacgtcaccagatgtggacctcggagatattagcggt
atcaatgccagtgttgtcaatattcagaaggaaatagaccgccttaatgaggtcgccaa
aaatctgaacgagagcctcatcgatcttcaggagctgggcaaatatgagcagtacatca
agtggccttggtatatttggcttggcttcatcgccggcctgatcgccatagtaatggtc
acaattatgctctctttatggatgtgctccaatggatcgttacaatgcagaatttgcat
ttaa
B.1.1.529 CoV S (GSAS-2P) AA
SEQ ID NO: 18
MAKNVAIFGLLFSLLVLVPSQIFASQCVNLTTRTQLPPAYTNSFTRGVYYPDKVERSSV
LHSTQDLFLPFFSNVTWFHVISGINGTKRFDNPVLPENDGVYFASIEKSNIIRGWIFGT
TLDSKTQSLLIVNNATNVVIKVCEFQFCNDPFLDHKNNKSWMESEFRVYSSANNCTFEY
VSQPFLMDLEGKQGNFKNLREFVFKNIDGYFKIYSKHTPIIVREPEDLPQGESALEPLV
DLPIGINITRFQTLLALHRSYLTPGDSSSGWTAGAAAYYVGYLQPRTELLKYNENGTIT
DAVDCALDPLSETKCTLKSFTVEKGIYQTSNFRVQPTESIVRFPNITNLCPFDEVENAT
RFASVYAWNRKRISNCVADYSVLYNLAPFFTFKCYGVSPTKLNDLCFTNVYADSFVIRG
DEVRQIAPGQTGNIADYNYKLPDDFTGCVIAWNSNKLDSKVSGNYNYLYRLFRKSNLKP
FERDISTEIYQAGNKPCNGVAGENCYFPLRSYSFRPTYGVGHQPYRVVVLSFELLHAPA
TVCGPKKSTNLVKNKCVNFNFNGLKGTGVLTESNKKELPFQQFGRDIADTTDAVRDPQT
LEILDITPCSFGGVSVITPGTNTSNQVAVLYQGVNCTEVPVAIHADQLTPTWRVYSTGS
NVFQTRAGCLIGAEYVNNSYECDIPIGAGICASYQTQTKSHGSASSVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSF
IEDLLENKVTLADAGFIKQYGDCLGDIAARDLICAQKFKGLTVLPPLLTDEMIAQYTSA
LLAGTITSGWTFGAGAALQIPFAMQMAYRENGIGVTQNVLYENQKLIANQFNSAIGKIQ
DSLSSTASALGKLQDVVNHNAQALNTLVKQLSSKFGAISSVLNDIFSRLDPPEAEVQID
RLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGFIAGLIAIVMV
TIMLSLWMCSNGSLQCRICI
B.1.1.529 CoV S (GSAS-2P+D253N) DNA
SEQ ID NO: 19
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatGT
Gatttccggcactaacggtactaagaggttcgataatcctgtgctccctttcaatgacg
gcgtttactttgcaagcATCgagaagagtaacatcatccgaggttggatctttggcact
accctcgattcaaagacgcagagcctcctcattgtgaacaatgccactaacgtggtgat
caaagtttgcgagtttcagttctgcaatgaccctttcTTGGACcataagaacaacaagt
cttggatggaatctgaattccgcgtctatagcagcgccaacaactgcacctttgaatac
gtgtcccagcccttccttatggacctggagggaaagcagggaaactttaagaatctgag
agagttcgtgtttaaaaatatcgacggctattttaagatctattctaagcacacgccta
ttATCgtgcgcGAGCCCGAGgatcttccacaaggcttcagcgccctggaaccactcgtg
gacctcccaattggtatcaacatcactagatttcagactctgcttgccctccaccgatc
ctatctgacacccggaAACtcctctagcggctggactgccggcgctgccgcttattacg
ttggttatcttcagccacgcacgttcctgctgaagtataacgagaatggtactattacc
gatgccgtggattgtgcccttgaccccctgtccgaaactaagtgcacactcaagtcatt
cactgtggaaaaaggaatctaccagacaagcaattttcgggtccagcctactgagagca
ttgtgcgctttcctaacatcacaaatctttgccccttcGACgaggttttcaatgctaca
cggtttgcctccgtgtatgcctggaaccgcaagagaatttccaattgcgtggccgatta
ctccgtgctctacaatCTGgcaCCCtttTTCacctttaagtgctatggcgtatccccta
ctaagcttaacgacttgtgtttcacaaacgtgtatgccgactcctttgtgatacggggc
gacgaagttagacagatagcaccaggacagacgggaAACatagctgactacaactataa
gcttcctgatgacttcactggctgcgttatcgcgtggaattctaacAAGctggactcaa
aagtcAGCggcaactataactatctctatcggctgttccgcaagagtaaccttaagccc
tttgagagagatataagcactgaaatctaccaggctggcAACAAGccctgtaatggcgt
gGCCggctttaattgttattttccactgAGAtcctatAGCtttAGAccaaccTACggcg
tgggcCACcaaccataccgcgtcgtggtgctctcctttgaactgctccacgctcccgcg
actgtctgcggccccaagaagtccacgaaccttgtgaagaataagtgcgttaattttaa
tttcaacggcctcAAGggaacaggagtgctcactgagagtaacaagaagttcctgccat
ttcaacaatttggcagagacatagccgatactactgacgccgttagggacccccagacc
ctcgagattctcgatataacgccctgctccttcggtggagtttccgtgatcacgccagg
caccaataccagtaaccaggtcgccgtgctgtatcagGGCgtcaactgtactgaggtgc
ccgtagccatccatgcggatcagctcacaccaacttggagggtgtacagcaccggctcc
aatgtattccagactcgggccggatgccttattggcgccgaaTACgtgaacaatagtta
cgaatgcgatattccaattggcgccggaatctgtgctagctaccagactcagacgAAGt
ccCACggcagcgccagcagcgttgccagccagtcaatcatcgcttatacaatgtcactt
ggagccgaaaactccgtggcttactcaaacaacagcatcgccatccccacaaacttcac
catatccgtgacaactgagattctgccagtgtccatgactaagacgtccgtagattgca
ctatgtacatatgcggcgacagcacagaatgttctaatctgctgctgcaatatggaagc
ttctgcactcaactgAAGagagcgctcacaggcatcgccgtggagcaggataagaatac
ccaggaggtgttcgcccaagttaagcagatctacaagaccccacccataaagTACttcg
gtggattcaattttagtcagatactcccagacccatctaagccatccaagaggagcttt
atcgaggatcttttgtttaacaaagttactctggccgacgccggtttcatcaagcagta
cggagattgcctcggcgacatcgctgctcgtgacctcatctgtgcgcaaaagtttAAGg
gtctgacggtgctgcctcccctccttactgatgaaatgatcgcccagtataccagcgca
ctcctcgctggcaccataacatccggttggacattcggcgctggtgcagcactgcagat
accattcgccatgcaaatggcatatcgtttcaacggtatcggtgtcacacagaatgtcc
tatatgagaaccagaagctgatcgcaaatcagttcaatagtgccatcggaaaaatccag
gatagccttagcagcacagcctcagcccttggcaaactccaggatgtcgtgaacCACaa
tgcccaggctctcaataccctcgtgaagcagctctcatctAAGttcggcgcaatttcca
gtgtcctcaacgacatcTTCagccgcctcgacccccccgaggccgaagtgcagattgac
agactgattacaggtcgactccagagcctccagacttacgtgactcagcagctgataag
agccgccgagataagggccagcgctaacctggctgccacaaagatgtctgagtgcgtgc
tgggccagtccaagagagtagacttctgtggcaaaggctaccatctgatgagcttccca
caatccgcacctcacggcgtagtgttcctccacgtgacatatgtaccggctcaggagaa
gaatttcactaccgctcctgctatatgccatgatggaaaggctcacttcccccgggagg
gggtgttcgtgtccaacggcacccattggtttgtgactcagcggaatttctacgaaccc
cagatcataaccactgacaacacatttgtgtccggaaattgtgacgtggtcattggaat
agtgaacaacactgtttatgatccactgcagccagaacttgacagctttaaggaggagc
tcgacaagtacttcaagaatcatacgtcaccagatgtggacctcggagatattagcggt
atcaatgccagtgttgtcaatattcagaaggaaatagaccgccttaatgaggtcgccaa
aaatctgaacgagagcctcatcgatcttcaggagctgggcaaatatgagcagtacatca
agtggccttggtatatttggcttggcttcatcgccggcctgatcgccatagtaatggtc
acaattatgctctctttatggatgtgctccaatggatcgttacaatgcagaatttgcat
ttaa
B.1.1.529 COV S (GSAS-2P+D253N) AA
SEQ ID NO: 20
MAKNVAIFGLLFSLLVLVPSQIFASQCVNLTTRTQLPPAYTNSFTRGVYYPDKVERSSV
LHSTQDLFLPFFSNVTWFHVISGTNGTKRFDNPVLPENDGVYFASIEKSNIIRGWIFGT
TLDSKTQSLLIVNNATNVVIKVCEFQFCNDPFLDHKNNKSWMESEFRVYSSANNCTFEY
VSQPFLMDLEGKQGNFKNLREFVFKNIDGYFKIYSKHTPIIVREPEDLPQGFSALEPLV
DLPIGINITRFQTLLALHRSYLTPGNSSSGWTAGAAAYYVGYLQPRTELLKYNENGTIT
DAVDCALDPLSETKCTLKSFTVEKGIYQTSNERVQPTESIVRFPNITNLCPFDEVENAT
RFASVYAWNRKRISNCVADYSVLYNLAPFFTFKCYGVSPTKLNDLCFTNVYADSFVIRG
DEVRQIAPGQTGNIADYNYKLPDDFTGCVIAWNSNKLDSKVSGNYNYLYRLFRKSNLKP
FERDISTEIYQAGNKPCNGVAGENCYFPLRSYSFRPTYGVGHQPYRVVVLSFELLHAPA
TVCGPKKSTNLVKNKCVNFNFNGLKGTGVLTESNKKFLPFQQFGRDIADTTDAVRDPQT
LEILDITPCSFGGVSVITPGTNTSNQVAVLYQGVNCTEVPVAIHADQLTPTWRVYSTGS
NVFQTRAGCLIGAEYVNNSYECDIPIGAGICASYQTQTKSHGSASSVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSF
IEDLLENKVTLADAGFIKQYGDCLGDIAARDLICAQKFKGLTVLPPLLTDEMIAQYTSA
LLAGTITSGWTFGAGAALQIPFAMQMAYRENGIGVTQNVLYENQKLIANQFNSAIGKIQ
DSLSSTASALGKLQDVVNHNAQALNTLVKQLSSKFGAISSVLNDIFSRLDPPEAEVQID
RLITGRLQSLQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGFIAGLIAIVMV
TIMLSLWMCSNGSLQCRICI
Cloning vector 8716 from left to right T-DNA
tggcaggatatattgtggtgtaaacaaattgacgcttagacaacttaataacacattgcg
SEQ ID NO: 21
gacgtttttaatgtactgaattaacgccgaatcccgggctggtatatttatatgttgtc
aaataactcaaaaaccataaaagtttaagttagcaagtgtgtacatttttacttgaaca
aaaatattcacctactactgttataaatcattattaaacattagagtaaagaaatatgg
atgataagaacaagagtagtgatattttgacaacaattttgttgcaacatttgagaaaa
ttttgttgttctctcttttcattggtcaaaaacaatagagagagaaaaaggaagaggga
gaataaaaacataatgtgagtatgagagagaaagttgtacaaaagttgtaccaaaatag
ttgtacaaatatcattgaggaatttgacaaaagctacacaaataagggttaattgctgt
aaataaataaggatgacgcattagagagatgtaccattagagaatttttggcaagtcat
taaaaagaaagaataaattatttttaaaattaaaagttgagtcatttgattaaacatgt
gattatttaatgaattgatgaaagagttggattaaagttgtattagtaattagaatttg
gtgtcaaatttaatttgacatttgatcttttcctatatattgccccatagagtcagtta
actcatttttatatttcatagatcaaataagagaaataacggtatattaatccctccaa
aaaaaaaaaacggtatatttactaaaaaatctaagccacgtaggaggataacaggatcc
ccgtaggaggataacatccaatccaaccaatcacaacaatcctgatgagataacccact
ttaagcccacgcatctgtggcacatctacattatctaaatcacacattcttccacacat
ctgagccacacaaaaaccaatccacatctttatcacccattctataaaaaatcacactt
tgtgagtctacactttgattcccttcaaacacatacaaagagaagagactaattaatta
attaatcatcttgagagaaaatggaacgagctatacaaggaaacgacgctagggaacaa
gctaacagtgaacgttgggatggaggatcaggaggtaccacttctcccttcaaacttcc
tgacgaaagtccgagttggactgagtggcggctacataacgatgagacgaattcgaatc
aagataatccccttggtttcaaggaaagctggggtttcgggaaagttgtatttaagaga
tatctcagatacgacaggacggaagcttcactgcacagagtccttggatcttggacggg
agattcggttaactatgcagcatctcgatttttcggtttcgaccagatcggatgtacct
atagtattcggtttcgaggagttagtatcaccgtttctggagggtcgcgaactcttcag
catctctgtgagatggcaattcggtctaagcaagaactgctacagcttgccccaatcga
agtggaaagtaatgtatcaagaggatgccctgaaggtactcaaaccttcgaaaaagaaa
gcgagtaagttaaaatgcttcttcgtctcctatttataatatggtttgttattgttaat
tttgttcttgtagaagagcttaattaatcgttgttgttatgaaatactatttgtatgag
atgaactggtgtaatgtaattcatttacataagtggagtcagaatcagaatgtttcctc
cataactaactagacatgaagacctgccgcgtacaattgtcttatatttgaacaactaa
aattgaacatcttttgccacaactttataagtggttaatatagctcaaatatatggtca
agttcaatagattaataatggaaatatcagttatcgaaattcattaacaatcaacttaa
cgttattaactactaattttatatcatcccctttgataaatgatagtacaccaattagg
aaggagcatgctcgcctaggagattgtcgtttcccgccttcagtttgcaagctgctcta
gccgtgtagccaatacgcaaaccgcctctccccgcgcgttgggaattactagcgcgtgt
cgacaagcttgcatgccggtcaacatggtggagcacgacacacttgtctactccaaaaa
tatcaaagatacagtctcagaagaccaaagggcaattgagacttttcaacaaagggtaa
tatccggaaacctcctcggattccattgcccagctatctgtcactttattgtgaagata
gtggaaaaggaaggtggctcctacaaatgccatcattgcgataaaggaaaggccatcgt
tgaagatgcctctgccgacagtggtcccaaagatggacccccacccacgaggagcatcg
tggaaaaagaagacgttccaaccacgtcttcaaagcaagtggattgatgtgataacatg
gtggagcacgacacacttgtctactccaaaaatatcaaagatacagtctcagaagacca
aagggcaattgagacttttcaacaaagggtaatatccggaaacctcctcggattccatt
gcccagctatctgtcactttattgtgaagatagtggaaaaggaaggtggctcctacaaa
tgccatcattgcgataaaggaaaggccatcgttgaagatgcctctgccgacagtggtcc
caaagatggacccccacccacgaggagcatcgtggaaaaagaagacgttccaaccacgt
cttcaaagcaagtggattgatgtgatatctccactgacgtaagggatgacgcacaatcc
cactatccttcgcaagacccttcctctatataaggaagttcatttcatttggagaggca
ctccatttgaatctatcaaaccaaaacacattgagcaaaatggcgaaaaacgttgcgat
tttcggcttattgttttctcttcttgtgttggttccttctcagatcttcgcgacgtcac
tcctcagccaaaacgacacccccatctgtctatccactggcccctggatctgctgccca
aactaactccatggtgaccctgggatgcctggtcaagggctatttccctgagccagtga
cagtgacctggaactctggatccctgtccagcggtgtgcacaccttcccagctgtcctg
cagtctgacctctacactctgagcagctcagtgactgtcccctccagcacctggcccag
cgagaccgtcacctgcaacgttgcccacccggccagcagcaccaaggtggacaagaaaa
ttgtgcccagggattgtggttgtaagccttgcatatgtacagtcccagaagtatcatct
gtcttcatcttccccccaaagcccaaggatgtgctcaccattactctgactcctaaggt
cacgtgtgttgtggtagacatcagcaaggatgatcccgaggtccagttcagctggtttg
tagatgatgtggaggtgcacacagctcagacgcaaccccgggaggagcagttcaacagc
actttccgctcagtcagtgaacttcccatcatgcaccaggactggctcaatggcaagga
gacgtccagattttggcgatctattcaactgtcgccagttcattggtactggtagtctc
cctgggggcaatcagtttctggatgtgctctaatgggtctctacagtgtagaatatgta
tttaaaggccttagtcgtgtcgtttttcaaataatataatccttttagggttttagtta
gtttaaattttctgttgctcctgtttagcaggtcgtgccttcagcaagcacacaaaaac
agagtgtttattttaagttgtttgtttagtgattcaaaaaaaaaatcgttcaaacattt
ggcaataaagtttcttaagattgaatcctgttgccggtcttgcgatgattatcatataa
tttctgttgaattacgttaagcatgtaataattaacatgtaatgcatgacgttatttat
gagatgggtttttatgattagagtcccgcaattatacatttaatacgcgatagaaaaca
aaatatagcgcgcaaactaggataaattatcgcgcgcggtgtcatctatgttactagat
ctctagagtctcaagcttggcgcgcccacgtgactagtggcactggccgtcgttttaca
acgtcgtgactgggaaaaccctggcgttacccaacttaatcgccttgcagcacatcccc
ctttcgccagctggcgtaatagcgaagaggcccgcaccgatcgcccttcccaacagttg
cgcagcctgaatggcgaatgctagagcagcttgagcttggatcagattgtcgtttcccg
ccttcagtttaaactatcagtgtttgacaggatatattggcgggtaaacctaagagaaa
agagcgttta
Construct 9125 from 2X35S prom to NOS term
SEQ ID NO: 22
gtcaacatggtggagcacgacacacttgtctactccaaaaatatcaaagatacagtctc
agaagaccaaagggcaattgagacttttcaacaaagggtaatatccggaaacctcctcg
gattccattgcccagctatctgtcactttattgtgaagatagtggaaaaggaaggtggc
tcctacaaatgccatcattgcgataaaggaaaggccatcgttgaagatgcctctgccga
cagtggtcccaaagatggacccccacccacgaggagcatcgtggaaaaagaagacgttc
caaccacgtcttcaaagcaagtggattgatgtgataacatggtggagcacgacacactt
gtctactccaaaaatatcaaagatacagtctcagaagaccaaagggcaattgagacttt
tcaacaaagggtaatatccggaaacctcctcggattccattgcccagctatctgtcact
ttattgtgaagatagtggaaaaggaaggtggctcctacaaatgccatcattgcgataaa
ggaaaggccatcgttgaagatgcctctgccgacagtggtcccaaagatggacccccacc
cacgaggagcatcgtggaaaaagaagacgttccaaccacgtcttcaaagcaagtggatt
gatgtgatatctccactgacgtaagggatgacgcacaatcccactatccttcgcaagac
ccttcctctatataaggaagttcatttcatttggagaggcactccatttgaatctatca
aaccaaaacacattgagcaaaatggcgaaaaacgttgcgattttcggcttattgttttc
tcttcttgtgttggttccttctcagatcttcgcgTCCCAATGCgtgaatcttacgacgc
gaacacagttaccacccgcatatacaaatagcttcactcggggtgtttattaccccgac
aaagtgttcaggtcctccgtgctccactcaacacaggacctctttcttcctttcttttc
taacgtgacatggtttcatgccattcatgtatccggcactaacggtactaagaggttcg
ataatcctgtgctccctttcaatgacggcgtttactttgcaagcacagagaagagtaac
atcatccgaggttggatctttggcactaccctcgattcaaagacgcagagcctcctcat
tgtgaacaatgccactaacgtggtgatcaaagtttgcgagtttcagttctgcaatgacc
ctttcttgggggtgtactatcataagaacaacaagtcttggatggaatctgaattccgc
gtctatagcagcgccaacaactgcacctttgaatacgtgtcccagcccttccttatgga
cctggagggaaagcagggaaactttaagaatctgagagagttcgtgtttaaaaatatcg
acggctattttaagatctattctaagcacacgcctattaatctcgtgcgcgatcttcca
caaggcttcagcgccctggaaccactcgtggacctcccaattggtatcaacatcactag
atttcagactctgcttgccctccaccgatcctatctgacacccggagactcctctagcg
gctggactgccggcgctgccgcttattacgttggttatcttcagccacgcacgttcctg
ctgaagtataacgagaatggtactattaccgatgccgtggattgtgcccttgaccccct
gtccgaaactaagtgcacactcaagtcattcactgtggaaaaaggaatctaccagacaa
gcaattttcgggtccagcctactgagagcattgtgcgctttcctaacatcacaaatctt
tgccccttcggagaggttttcaatgctacacggtttgcctccgtgtatgcctggaaccg
caagagaatttccaattgcgtggccgattactccgtgctctacaatagtgcaagcttta
gcacctttaagtgctatggcgtatcccctactaagcttaacgacttgtgtttcacaaac
gtgtatgccgactcctttgtgatacggggcgacgaagttagacagatagcaccaggaca
gacgggaaagatagctgactacaactataagcttcctgatgacttcactggctgcgtta
tcgcgtggaattctaacaacctggactcaaaagtcggcggcaactataactatctctat
cggctgttccgcaagagtaaccttaagccctttgagagagatataagcactgaaatcta
ccaggctggcagtacgccctgtaatggcgtggaaggctttaattgttattttccactgc
aatcctatggttttcagccaaccaatggcgtgggctaccaaccataccgcgtcgtggtg
ctctcctttgaactgctccacgctcccgcgactgtctgcggccccaagaagtccacgaa
ccttgtgaagaataagtgcgttaattttaatttcaacggcctcactggaacaggagtgc
tcactgagagtaacaagaagttcctgccatttcaacaatttggcagagacatagccgat
actactgacgccgttagggacccccagaccctcgagattctcgatataacgccctgctc
cttcggtggagtttccgtgatcacgccaggcaccaataccagtaaccaggtcgccgtgc
tgtatcaggatgtcaactgtactgaggtgcccgtagccatccatgcggatcagctcaca
ccaacttggagggtgtacagcaccggctccaatgtattccagactcgggccggatgcct
tattggcgccgaacacgtgaacaatagttacgaatgcgatattccaattggcgccggaa
tctgtgctagctaccagactcagacgaactccccaggcagcgccagcagcgttgccagc
cagtcaatcatcgcttatacaatgtcacttggagccgaaaactccgtggcttactcaaa
caacagcatcgccatccccacaaacttcaccatatccgtgacaactgagattctgccag
tgtccatgactaagacgtccgtagattgcactatgtacatatgcggcgacagcacagaa
tgttctaatctgctgctgcaatatggaagcttctgcactcaactgaacagagcgctcac
aggcatcgccgtggagcaggataagaatacccaggaggtgttcgcccaagttaagcaga
tctacaagaccccacccataaaggatttcggtggattcaattttagtcagatactccca
gacccatctaagccatccaagaggagctttatcgaggatcttttgtttaacaaagttac
tctggccgacgccggtttcatcaagcagtacggagattgcctcggcgacatcgctgctc
gtgacctcatctgtgcgcaaaagtttaacggtctgacggtgctgcctcccctccttact
gatgaaatgatcgcccagtataccagcgcactcctcgctggcaccataacatccggttg
gacattcggcgctggtgcagcactgcagataccattcgccatgcaaatggcatatcgtt
tcaacggtatcggtgtcacacagaatgtcctatatgagaaccagaagctgatcgcaaat
cagttcaatagtgccatcggaaaaatccaggatagccttagcagcacagcctcagccct
tggcaaactccaggatgtcgtgaaccagaatgcccaggctctcaataccctcgtgaagc
agctctcatctaatttcggcgcaatttccagtgtcctcaacgacatcctcagccgcctc
gacccccccgaggccgaagtgcagattgacagactgattacaggtcgactccagagcct
ccagacttacgtgactcagcagctgataagagccgccgagataagggccagcgctaacc
tggctgccacaaagatgtctgagtgcgtgctgggccagtccaagagagtagacttctgt
ggcaaaggctaccatctgatgagcttcccacaatccgcacctcacggcgtagtgttcct
ccacgtgacatatgtaccggctcaggagaagaatttcactaccgctcctgctatatgcc
atgatggaaaggctcacttcccccgggagggggtgttcgtgtccaacggcacccattgg
tttgtgactcagcggaatttctacgaaccccagatcataaccactgacaacacatttgt
gtccggaaattgtgacgtggtcattggaatagtgaacaacactgtttatgatccactgc
agccagaacttgacagctttaaggaggagctcgacaagtacttcaagaatcatacgtca
ccagatgtggacctcggagatattagcggtatcaatgccagtgttgtcaatattcagaa
ggaaatagaccgccttaatgaggtcgccaaaaatctgaacgagagcctcatcgatcttc
aggagctgggcaaatatgagcagtacatcaagtggccttggtatatttggcttggcttc
atcgccggcctgatcgccatagtaatggtcacaattatgctctctttatggatgtgctc
caatggatcgttacaatgcagaatttgcatttaaaggccttagtcgtgtcgtttttcaa
ataatataatccttttagggttttagttagtttaaattttctgttgctcctgtttagca
ggtcgtgccttcagcaagcacacaaaaacagagtgtttattttaagttgtttgtttagt
gattcaaaaaaaaaatcgttcaaacatttggcaataaagtttcttaagattgaatcctg
ttgccggtcttgcgatgattatcatataatttctgttgaattacgttaagcatgtaata
attaacatgtaatgcatgacgttatttatgagatgggtttttatgattagagtcccgca
attatacatttaatacgcgatagaaaacaaaatatagcgcgcaaactaggataaattat
cgcgcgcggtgtcatctatgttactagat
IF(PDI)S-3-NY-CoV.c
SEQ ID NO: 23
TCTCAGATCTTCGCGTCCCAATGCGTGAATCTTACGACGCGAACACAGT
IF(Avb)-H5I.r
SEQ ID NO: 24
acgacacgactaaggcctttaaatgcaaattctgcattgtaacgatcc
Mature B CoV S (GSAS-2P+del246-252) AA without SP
SEQ ID NO: 25
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHDSS
SGWTAGAAAYYVGYLQPRTFLLKYNENGTITDAVDCALDPLSETKCTLKSFTVEKGIYQ
TSNFRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGC
VIAWNSNNLDSKVGGNYNYLYRLERKSNLKPFERDISTEIYQAGSTPCNGVEGENCYFP
LQSYGFQPTNGVGYQPYRVVVLSFELLHAPATVCGPKKSTNLVKNKCVNFNENGLTGTG
VLTESNKKELPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIPIGA
GICASYQTQTNSPGSASSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSFIEDLLENKVTLADAGFIKQYGDCLGDIA
ARDLICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALNTLV
KQLSSNFGAISSVLNDILSRLDPPEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLID
LQELGKYEQYIKWPWYIWLGFIAGLIAIVMVTIMLSLWMCSNGSLQCRICI*
Mature B CoV S (GSAS-2P+D253N) AA without SP
SEQ ID NO: 26
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRSY
LTPGNSSSGWTAGAAAYYVGYLQPRTELLKYNENGTITDAVDCALDPLSETKCTLKSFT
VEKGIYQTSNERVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIADYNYKL
PDDFTGCVIAWNSNNLDSKVGGNYNYLYRLERKSNLKPFERDISTEIYQAGSTPCNGVE
GENCYFPLQSYGFQPTNGVGYQPYRVVVLSFELLHAPATVCGPKKSTNLVKNKCVNFNF
NGLTGTGVLTESNKKELPFQQFGRDIADTTDAVRDPQTLEILDITPCSFGGVSVITPGT
NTSNQVAVLYQDVNCTEVPVAIHADQLTPTWRVYSTGSNVFQTRAGCLIGAEHVNNSYE
CDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQ
EVFAQVKQIYKTPPIKDFGGFNFSQILPDPSKPSKRSFIEDLLENKVTLADAGFIKQYG
DCLGDIAARDLICAQKENGLTVLPPLLTDEMIAQYTSALLAGTITSGWTFGAGAALQIP
FAMQMAYRENGIGVTQNVLYENQKLIANQFNSAIGKIQDSLSSTASALGKLQDVVNQNA
QALNTLVKQLSSNFGAISSVLNDILSRLDPPEAEVQIDRLITGRLQSLQTYVTQQLIRA
AEIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKN
LNESLIDLQELGKYEQYIKWPWYIWLGFIAGLIAIVMVTIMLSLWMCSNGSLQCRICI
Mature B CoV S (GSAS-2P+del246-252+D253N) AA without SP
SEQ ID NO: 27
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHNSS
SGWTAGAAAYYVGYLQPRTELLKYNENGTITDAVDCALDPLSETKCTLKSFTVEKGIYQ
TSNFRVQPTESIVRFPNITNLCPFGEVENATRFASVYAWNRKRISNCVADYSVLYNSAS
FSTFKCYGVSPTKLNDLCFTNVYADSFVIRGDEVRQIAPGQTGKIADYNYKLPDDFTGC
VIAWNSNNLDSKVGGNYNYLYRLERKSNLKPFERDISTEIYQAGSTPCNGVEGENCYFP
LQSYGFQPTNGVGYQPYRVVVLSFELLHAPATVCGPKKSTNLVKNKCVNFNFNGLTGTG
VLTESNKKELPFQQFGRDIADTTDAVRDPQTLEILDITPCSFGGVSVITPGTNTSNQVA
VLYQDVNCTEVPVAIHADQLTPTWRVYSTGSNVFQTRAGCLIGAEHVNNSYECDIPIGA
GICASYQTQTNSPGSASSVASQSIIAYTMSLGAENSVAYSNNSIAIPTNFTISVTTEIL
PVSMTKTSVDCTMYICGDSTECSNLLLQYGSFCTQLNRALTGIAVEQDKNTQEVFAQVK
QIYKTPPIKDFGGFNFSQILPDPSKPSKRSFIEDLLENKVTLADAGFIKQYGDCLGDIA
ARDLICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALNTLV
KQLSSNFGAISSVLNDILSRLDPPEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLID
LQELGKYEQYIKWPWYIWLGFIAGLIAIVMVTIMLSLWMCSNGSLQCRICI
Mature B CoV S (GSAS-2P+S13+G252N) AA without SP
SEQ ID NO: 28
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRSY
LTPNDSSSGWTAGAAAYYVGYLQPRTFLLKYNENGTITDAVDCALDPLSETKCTLKSFT
VEKGIYQTSNERVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIADYNYKL
PDDFTGCVIAWNSNNLDSKVGGNYNYLYRLERKSNLKPFERDISTEIYQAGSTPCNGVE
GENCYFPLQSYGFQPTNGVGYQPYRVVVLSFELLHAPATVCGPKKSTNLVKNKCVNFNF
NGLTGTGVLTESNKKELPFQQFGRDIADTTDAVRDPQTLEILDITPCSFGGVSVITPGT
NTSNQVAVLYQDVNCTEVPVAIHADQLTPTWRVYSTGSNVFQTRAGCLIGAEHVNNSYE
CDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQ
EVFAQVKQIYKTPPIKDFGGFNFSQILPDPSKPSKRSFIEDLLENKVTLADAGFIKQYG
DCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQNA
QALNTLVKQLSSNFGAISSVLNDILSRLDPPEAEVQIDRLITGRLQSLQTYVTQQLIRA
AEIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKN
LNESLIDLQELGKYEQYIKWPWYIWLGFIAGLIAIVMVTIMLSLWMCSNGSLQCRICI
Mature B CoV S (GSAS-2P+S13+P251N+D253T) AA without SP
SEQ ID NO: 29
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRSY
LTNGTSSSGWTAGAAAYYVGYLQPRTFLLKYNENGTITDAVDCALDPLSETKCTLKSFT
VEKGIYQTSNERVQPTESIVRFPNITNLCPFGEVENATRFASVYAWNRKRISNCVADYS
VLYNSASFSTFKCYGVSPTKLNDLCFTNVYADSFVIRGDEVRQIAPGQTGKIADYNYKL
PDDFTGCVIAWNSNNLDSKVGGNYNYLYRLFRKSNLKPFERDISTEIYQAGSTPCNGVE
GENCYFPLQSYGFQPTNGVGYQPYRVVVLSFELLHAPATVCGPKKSTNLVKNKCVNFNF
NGLTGTGVLTESNKKELPFQQFGRDIADTTDAVRDPQTLEILDITPCSFGGVSVITPGT
NTSNQVAVLYQDVNCTEVPVAIHADQLTPTWRVYSTGSNVFQTRAGCLIGAEHVNNSYE
CDIPIGAGICASYQTQTNSPGSASSVASQSIIAYTMSLGAENSVAYSNNSIAIPTNFTI
SVTTEILPVSMTKTSVDCTMYICGDSTECSNLLLQYGSFCTQLNRALTGIAVEQDKNTQ
EVFAQVKQIYKTPPIKDFGGFNFSQILPDPSKPSKRSFIEDLLENKVTLADAGFIKQYG
DCLGDIAARDLICAQKENGLTVLPPLLTDEMIAQYTSALLAGTITSGWTFGAGAALQIP
FAMQMAYRENGIGVTQNVLYENQKLIANQFNSAIGKIQDSLSSTASALGKLQDVVNQNA
QALNTLVKQLSSNFGAISSVLNDILSRLDPPEAEVQIDRLITGRLQSLQTYVTQQLIRA
AEIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKEIDRLNEVAKN
LNESLIDLQELGKYEQYIKWPWYIWLGFIAGLIAIVMVTIMLSLWMCSNGSLQCRICI
Mature B CoV S (GSAS-2P+S13+Δ247-250) AA without SP
SEQ ID NO: 30
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRPG
DSSSGWTAGAAAYYVGYLQPRIFLLKYNENGTITDAVDCALDPLSETKCTLKSFTVEKG
IYQTSNFRVQPTESIVRFPNITNLCPFGEVENATRFASVYAWNRKRISNCVADYSVLYN
SASFSTFKCYGVSPTKLNDLCFTNVYADSFVIRGDEVRQIAPGQTGKIADYNYKLPDDE
TGCVIAWNSNNLDSKVGGNYNYLYRLFRKSNLKPFERDISTEIYQAGSTPCNGVEGENC
YFPLQSYGFQPTNGVGYQPYRVVVLSFELLHAPATVCGPKKSTNLVKNKCVNFNENGLT
GTGVLTESNKKELPFQQFGRDIADTTDAVRDPQTLEILDITPCSFGGVSVITPGTNTSN
QVAVLYQDVNCTEVPVAIHADQLTPTWRVYSTGSNVFQTRAGCLIGAEHVNNSYECDIP
IGAGICASYQTQTNSPGSASSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLENKVTLADAGFIKQYGDCLG
DIAARDLICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVNQNAQALN
TLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNES
LIDLQELGKYEQYIKWPWYIWLGFIAGLIAIVMVTIMLSLWMCSNGSLQCRICI
Mature B CoV S (GSAS-2P+S13+Δ248-251) AA without SP x
SEQ ID NO: 31
SQCVNLTTRTQLPPAYTNSFTRGVYYPDKVERSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRSG
DSSSGWTAGAAAYYVGYLQPRTFLLKYNENGTITDAVDCALDPLSETKCTLKSFTVEKG
IYQTSNFRVQPTESIVRFPNITNLCPFGEVENATRFASVYAWNRKRISNCVADYSVLYN
SASFSTFKCYGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTPCNGVEGENC
YFPLQSYGFQPTNGVGYQPYRVVVLSFELLHAPATVCGPKKSTNLVKNKCVNFNENGLT
GTGVLTESNKKELPFQQFGRDIADTTDAVRDPQTLEILDITPCSFGGVSVITPGTNTSN
QVAVLYQDVNCTEVPVAIHADQLTPTWRVYSTGSNVFQTRAGCLIGAEHVNNSYECDIP
IGAGICASYQTQTNSPGSASSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLG
DIAARDLICAQKENGLTVLPPLLTDEMIAQYTSALLAGTITSGWTFGAGAALQIPFAMQ
MAYRENGIGVTQNVLYENQKLIANQFNSAIGKIQDSLSSTASALGKLQDVVNQNAQALN
TLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNES
LIDLQELGKYEQYIKWPWYIWLGFIAGLIAIVMVTIMLSLWMCSNGSLQCRICI
Mature B CoV S (GSAS-2P+S13+Δ249-252) AA without SP
SEQ ID NO: 32
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRSY
DSSSGWTAGAAAYYVGYLQPRTELLKYNENGTITDAVDCALDPLSETKCTLKSFTVEKG
IYQTSNFRVQPTESIVREPNITNLCPFGEVENATRFASVYAWNRKRISNCVADYSVLYN
SASFSTFKCYGVSPTKLNDLCFTNVYADSFVIRGDEVRQIAPGQTGKIADYNYKLPDDF
TGCVIAWNSNNLDSKVGGNYNYLYRLFRKSNLKPFERDISTEIYQAGSTPCNGVEGENC
YFPLQSYGFQPTNGVGYQPYRVVVLSFELLHAPATVCGPKKSTNLVKNKCVNFNENGLT
GTGVLTESNKKFLPFQQFGRDIADTTDAVRDPQTLEILDITPCSFGGVSVITPGTNTSN
QVAVLYQDVNCTEVPVAIHADQLTPTWRVYSTGSNVFQTRAGCLIGAEHVNNSYECDIP
IGAGICASYQTQTNSPGSASSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLENKVTLADAGFIKQYGDCLG
DIAARDLICAQKFNGLTVLPPLLTDEMIAQYTSALLAGTITSGWTFGAGAALQIPFAMQ
MAYRENGIGVTQNVLYENQKLIANQFNSAIGKIQDSLSSTASALGKLQDVVNQNAQALN
TLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNES
LIDLQELGKYEQYIKWPWYIWLGFIAGLIAIVMVTIMLSLWMCSNGSLQCRICI
Mature B CoV S (GSAS-2P+S13+Δ246-250) AA without SP
SEQ ID NO: 33
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNL
REFVFKNIDGYFKIYSKHTPINLVRDLPQGFSALEPLVDLPIGINITRFQTLLALHPGD
SSSGWTAGAAAYYVGYLQPRTFLLKYNENGTITDAVDCALDPLSETKCTLKSFTVEKGI
YQTSNFRVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYKLPDDFT
GCVIAWNSNNLDSKVGGNYNYLYRLERKSNLKPFERDISTEIYQAGSTPCNGVEGENCY
FPLQSYGFQPTNGVGYQPYRVVVLSFELLHAPATVCGPKKSTNLVKNKCVNENENGLTG
TGVLTESNKKELPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNSYECDIPI
GAGICASYQTQTNSPGSASSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGFIKQYGDCLGD
IAARDLICAQKFNGLTVLPPLLTDEMIAQYTSALLAGTITSGWTFGAGAALQIPFAMQM
AYRENGIGVTQNVLYENQKLIANQFNSAIGKIQDSLSSTASALGKLQDVVNQNAQALNT
LVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESL
IDLQELGKYEQYIKWPWYIWLGFIAGLIAIVMVTIMLSLWMCSNGSLQCRICI*
Mature B CoV S (GSAS-2P+S13+Δ247-251) AA without SP
SEQ ID NO: 34
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRGD
SSSGWTAGAAAYYVGYLQPRTELLKYNENGTITDAVDCALDPLSETKCTLKSFTVEKGI
YQTSNERVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYKLPDDET
GCVIAWNSNNLDSKVGGNYNYLYRLERKSNLKPFERDISTEIYQAGSTPCNGVEGENCY
FPLQSYGFQPTNGVGYQPYRVVVLSFELLHAPATVCGPKKSTNLVKNKCVNFNENGLTG
TGVLTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNSYECDIPI
GAGICASYQTQTNSPGSASSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGFIKQYGDCLGD
IAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNT
LVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESL
IDLQELGKYEQYIKWPWYIWLGFIAGLIAIVMVTIMLSLWMCSNGSLQCRICI
Mature B CoV S (GSAS-2P+S13+Δ248-252) AA without SP
SEQ ID NO: 35
SQCVNLTTRTQLPPAYTNSFTRGVYYPDKVFRSSVLHSTQDLFLPFFSNVTWFHAIHVS
GTNGTKRFDNPVLPENDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNFKNL
REFVFKNIDGYFKIYSKHTPINLVRDLPQGFSALEPLVDLPIGINITRFQTLLALHRSD
SSSGWTAGAAAYYVGYLQPRTELLKYNENGTITDAVDCALDPLSETKCTLKSFTVEKGI
YQTSNFRVQPTESIVRFPNITNLCPFGEVENATRFASVYAWNRKRISNCVADYSVLYNS
ASFSTFKCYGVSPTKLNDLCFTNVYADSFVIRGDEVRQIAPGQTGKIADYNYKLPDDET
GCVIAWNSNNLDSKVGGNYNYLYRLERKSNLKPFERDISTEIYQAGSTPCNGVEGENCY
FPLQSYGFQPTNGVGYQPYRVVVLSFELLHAPATVCGPKKSTNLVKNKCVNFNENGLTG
TGVLTESNKKFLPFQQFGRDIADTTDAVRDPQTLEILDITPCSFGGVSVITPGTNTSNQ
VAVLYQDVNCTEVPVAIHADQLTPTWRVYSTGSNVFQTRAGCLIGAEHVNNSYECDIPI
GAGICASYQTQTNSPGSASSVASQSIIAYTMSLGAENSVAYSNNSIAIPTNFTISVTTE
ILPVSMTKTSVDCTMYICGDSTECSNLLLQYGSFCTQLNRALTGIAVEQDKNTQEVFAQ
VKQIYKTPPIKDFGGFNFSQILPDPSKPSKRSFIEDLLENKVTLADAGFIKQYGDCLGD
IAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNT
LVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESL
IDLQELGKYEQYIKWPWYIWLGFIAGLIAIVMVTIMLSLWMCSNGSLQCRICI
Mature B CoV S (GSAS-2P+S13+Δ246-251) AA without SP
SEQ ID NO: 36
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHGDS
SSGWTAGAAAYYVGYLQPRTELLKYNENGTITDAVDCALDPLSETKCTLKSFTVEKGIY
QTSNFRVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDFTG
CVIAWNSNNLDSKVGGNYNYLYRLERKSNLKPFERDISTEIYQAGSTPCNGVEGENCYF
PLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNNSYECDIPIG
AGICASYQTQTNSPGSASSVASQSIIAYTMSLGAENSVAYSNNSIAIPTNFTISVTTEI
LPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLENKVTLADAGFIKQYGDCLGDI
AARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNQNAQALNTL
VKQLSSNFGAISSVLNDILSRLDPPEAEVQIDRLITGRLQSLQTYVTQQLIRAAEIRAS
ANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYD
PLQPELDSFKEELDKYFKNHTSPDVDLGDISGINASVVNIQKEIDRLNEVAKNLNESLI
DLQELGKYEQYIKWPWYIWLGFIAGLIAIVMVTIMLSLWMCSNGSLQCRICI*
Mature B CoV S (GSAS-2P+S13+Δ247-252) AA without SP
SEQ ID NO: 37
SQCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKV
CEFQFCNDPFLGVYYHKNNKSWMESEFRVYSSANNCTFEYVSQPFLMDLEGKQGNEKNL
REFVFKNIDGYFKIYSKHTPINLVRDLPQGFSALEPLVDLPIGINITRFQTLLALHRDS
SSGWTAGAAAYYVGYLQPRTELLKYNENGTITDAVDCALDPLSETKCTLKSFTVEKGIY
QTSNERVQPTESIVRFPNITNLCPFGEVENATRFASVYAWNRKRISNCVADYSVLYNSA
SFSTFKCYGVSPTKLNDLCFTNVYADSFVIRGDEVRQIAPGQTGKIADYNYKLPDDETG
CVIAWNSNNLDSKVGGNYNYLYRLERKSNLKPFERDISTEIYQAGSTPCNGVEGENCYF
PLQSYGFQPTNGVGYQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQV
AVLYQDVNCTEVPVAIHADQLTPTWRVYSTGSNVFQTRAGCLIGAEHVNNSYECDIPIG
AGICASYQTQTNSPGSASSVASQSIIAYTMSLGAENSVAYSNNSIAIPTNFTISVTTEI
LPVSMTKTSVDCTMYICGDSTECSNLLLQYGSFCTQLNRALTGIAVEQDKNTQEVFAQV
KQIYKTPPIKDFGGFNFSQILPDPSKPSKRSFIEDLLENKVTLADAGFIKQYGDCLGDI
AARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNQNAQALNTL
VKQLSSNFGAISSVLNDILSRLDPPEAEVQIDRLITGRLQSLQTYVTQQLIRAAEIRAS
ANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYD
PLQPELDSFKEELDKYFKNHTSPDVDLGDISGINASVVNIQKEIDRLNEVAKNLNESLI
DLQELGKYEQYIKWPWYIWLGFIAGLIAIVMVTIMLSLWMCSNGSLQCRICI*
B CoV S (GSAS-2P+S13+G252N) DNA
SEQ ID NO: 38
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatcctatctgacacccAATgactcctctagcggctggactgccggcgctgccg
cttattacgttggttatcttcagccacgcacgttcctgctgaagtataacgagaatggt
actattaccgatgccgtggattgtgcccttgaccccctgtccgaaactaagtgcacact
caagtcattcactgtggaaaaaggaatctaccagacaagcaattttcgggtccagccta
ctgagagcattgtgcgctttcctaacatcacaaatctttgccccttcggagaggttttc
aatgctacacggtttgcctccgtgtatgcctggaaccgcaagagaatttccaattgcgt
ggccgattactccgtgctctacaatagtgcaagctttagcacctttaagtgctatggcg
tatcccctactaagcttaacgacttgtgtttcacaaacgtgtatgccgactcctttgtg
atacggggcgacgaagttagacagatagcaccaggacagacgggaaagatagctgacta
caactataagcttcctgatgacttcactggctgcgttatcgcgtggaattctaacaacc
tggactcaaaagtcggcggcaactataactatctctatcggctgttccgcaagagtaac
cttaagccctttgagagagatataagcactgaaatctaccaggctggcagtacgccctg
taatggcgtggaaggctttaattgttattttccactgcaatcctatggttttcagccaa
ccaatggcgtgggctaccaaccataccgcgtcgtggtgctctcctttgaactgctccac
gctcccgcgactgtctgcggccccaagaagtccacgaaccttgtgaagaataagtgcgt
taattttaatttcaacggcctcactggaacaggagtgctcactgagagtaacaagaagt
tcctgccatttcaacaatttggcagagacatagccgatactactgacgccgttagggac
ccccagaccctcgagattctcgatataacgccctgctccttcggtggagtttccgtgat
cacgccaggcaccaataccagtaaccaggtcgccgtgctgtatcaggatgtcaactgta
ctgaggtgcccgtagccatccatgcggatcagctcacaccaacttggagggtgtacagc
accggctccaatgtattccagactcgggccggatgccttattggcgccgaacacgtgaa
caatagttacgaatgcgatattccaattggcgccggaatctgtgctagctaccagactc
agacgaactccccaggcagcgccagcagcgttgccagccagtcaatcatcgcttataca
atgtcacttggagccgaaaactccgtggcttactcaaacaacagcatcgccatccccac
aaacttcaccatatccgtgacaactgagattctgccagtgtccatgactaagacgtccg
tagattgcactatgtacatatgcggcgacagcacagaatgttctaatctgctgctgcaa
tatggaagcttctgcactcaactgaacagagcgctcacaggcatcgccgtggagcagga
taagaatacccaggaggtgttcgcccaagttaagcagatctacaagaccccacccataa
aggatttcggtggattcaattttagtcagatactcccagacccatctaagccatccaag
aggagctttatcgaggatcttttgtttaacaaagttactctggccgacgccggtttcat
caagcagtacggagattgcctcggcgacatcgctgctcgtgacctcatctgtgcgcaaa
agtttaacggtctgacggtgctgcctcccctccttactgatgaaatgatcgcccagtat
accagcgcactcctcgctggcaccataacatccggttggacattcggcgctggtgcagc
actgcagataccattcgccatgcaaatggcatatcgtttcaacggtatcggtgtcacac
agaatgtcctatatgagaaccagaagctgatcgcaaatcagttcaatagtgccatcgga
aaaatccaggatagccttagcagcacagcctcagcccttggcaaactccaggatgtcgt
gaaccagaatgcccaggctctcaataccctcgtgaagcagctctcatctaatttcggcg
caatttccagtgtcctcaacgacatcctcagccgcctcgacccccccgaggccgaagtg
cagattgacagactgattacaggtcgactccagagcctccagacttacgtgactcagca
gctgataagagccgccgagataagggccagcgctaacctggctgccacaaagatgtctg
agtgcgtgctgggccagtccaagagagtagacttctgtggcaaaggctaccatctgatg
agcttcccacaatccgcacctcacggcgtagtgttcctccacgtgacatatgtaccggc
tcaggagaagaatttcactaccgctcctgctatatgccatgatggaaaggctcacttcc
cccgggagggggtgttcgtgtccaacggcacccattggtttgtgactcagcggaatttc
tacgaaccccagatcataaccactgacaacacatttgtgtccggaaattgtgacgtggt
cattggaatagtgaacaacactgtttatgatccactgcagccagaacttgacagcttta
aggaggagctcgacaagtacttcaagaatcatacgtcaccagatgtggacctcggagat
attagcggtatcaatgccagtgttgtcaatattcagaaggaaatagaccgccttaatga
ggtcgccaaaaatctgaacgagagcctcatcgatcttcaggagctgggcaaatatgagc
agtacatcaagtggccttggtatatttggcttggcttcatcgccggcctgatcgccata
gtaatggtcacaattatgctctctttatggatgtgctccaatggatcgttacaatgcag
aatttgcatttaa
B CoV S (GSAS-2P+S13+G252N) AA
SEQ ID NO: 39
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRSYLTPNDSSSGWTAGAAAYYVGYLQPRTFLLKYNENG
TITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELLH
APATVCGPKKSTNLVKNKCVNFNFNGLTGTGVLTESNKKELPFQQFGRDIADTTDAVRD
PQTLEILDITPCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPSK
RSFIEDLLENKVTLADAGFIKQYGDCLGDIAARDLICAQKENGLTVLPPLLTDEMIAQY
TSALLAGTITSGWTFGAGAALQIPFAMQMAYRENGIGVTQNVLYENQKLIANQFNSAIG
KIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQRNE
YEPQIITTDNTFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIWLGFIAGLIAI
VMVTIMLSLWMCSNGSLQCRICI
B CoV S (GSAS-2P+S13+P251N+D253T) DNA
SEQ ID NO: 40
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatcctatctgacaAATggaACCtcctctagcggctggactgccggcgctgccg
cttattacgttggttatcttcagccacgcacgttcctgctgaagtataacgagaatggt
actattaccgatgccgtggattgtgcccttgaccccctgtccgaaactaagtgcacact
caagtcattcactgtggaaaaaggaatctaccagacaagcaattttcgggtccagccta
ctgagagcattgtgcgctttcctaacatcacaaatctttgccccttcggagaggttttc
aatgctacacggtttgcctccgtgtatgcctggaaccgcaagagaatttccaattgcgt
ggccgattactccgtgctctacaatagtgcaagctttagcacctttaagtgctatggcg
tatcccctactaagcttaacgacttgtgtttcacaaacgtgtatgccgactcctttgtg
atacggggcgacgaagttagacagatagcaccaggacagacgggaaagatagctgacta
caactataagcttcctgatgacttcactggctgcgttatcgcgtggaattctaacaacc
tggactcaaaagtcggcggcaactataactatctctatcggctgttccgcaagagtaac
cttaagccctttgagagagatataagcactgaaatctaccaggctggcagtacgccctg
taatggcgtggaaggctttaattgttattttccactgcaatcctatggttttcagccaa
ccaatggcgtgggctaccaaccataccgcgtcgtggtgctctcctttgaactgctccac
gctcccgcgactgtctgcggccccaagaagtccacgaaccttgtgaagaataagtgcgt
taattttaatttcaacggcctcactggaacaggagtgctcactgagagtaacaagaagt
tcctgccatttcaacaatttggcagagacatagccgatactactgacgccgttagggac
ccccagaccctcgagattctcgatataacgccctgctccttcggtggagtttccgtgat
cacgccaggcaccaataccagtaaccaggtcgccgtgctgtatcaggatgtcaactgta
ctgaggtgcccgtagccatccatgcggatcagctcacaccaacttggagggtgtacagc
accggctccaatgtattccagactcgggccggatgccttattggcgccgaacacgtgaa
caatagttacgaatgcgatattccaattggcgccggaatctgtgctagctaccagactc
agacgaactccccaggcagcgccagcagcgttgccagccagtcaatcatcgcttataca
atgtcacttggagccgaaaactccgtggcttactcaaacaacagcatcgccatccccac
aaacttcaccatatccgtgacaactgagattctgccagtgtccatgactaagacgtccg
tagattgcactatgtacatatgcggcgacagcacagaatgttctaatctgctgctgcaa
tatggaagcttctgcactcaactgaacagagcgctcacaggcatcgccgtggagcagga
taagaatacccaggaggtgttcgcccaagttaagcagatctacaagaccccacccataa
aggatttcggtggattcaattttagtcagatactcccagacccatctaagccatccaag
aggagctttatcgaggatcttttgtttaacaaagttactctggccgacgccggtttcat
caagcagtacggagattgcctcggcgacatcgctgctcgtgacctcatctgtgcgcaaa
agtttaacggtctgacggtgctgcctcccctccttactgatgaaatgatcgcccagtat
accagcgcactcctcgctggcaccataacatccggttggacattcggcgctggtgcagc
actgcagataccattcgccatgcaaatggcatatcgtttcaacggtatcggtgtcacac
agaatgtcctatatgagaaccagaagctgatcgcaaatcagttcaatagtgccatcgga
aaaatccaggatagccttagcagcacagcctcagcccttggcaaactccaggatgtcgt
gaaccagaatgcccaggctctcaataccctcgtgaagcagctctcatctaatttcggcg
caatttccagtgtcctcaacgacatcctcagccgcctcgacccccccgaggccgaagtg
cagattgacagactgattacaggtcgactccagagcctccagacttacgtgactcagca
gctgataagagccgccgagataagggccagcgctaacctggctgccacaaagatgtctg
agtgcgtgctgggccagtccaagagagtagacttctgtggcaaaggctaccatctgatg
agcttcccacaatccgcacctcacggcgtagtgttcctccacgtgacatatgtaccggc
tcaggagaagaatttcactaccgctcctgctatatgccatgatggaaaggctcacttcc
cccgggagggggtgttcgtgtccaacggcacccattggtttgtgactcagcggaatttc
tacgaaccccagatcataaccactgacaacacatttgtgtccggaaattgtgacgtggt
cattggaatagtgaacaacactgtttatgatccactgcagccagaacttgacagcttta
aggaggagctcgacaagtacttcaagaatcatacgtcaccagatgtggacctcggagat
attagcggtatcaatgccagtgttgtcaatattcagaaggaaatagaccgccttaatga
ggtcgccaaaaatctgaacgagagcctcatcgatcttcaggagctgggcaaatatgagc
agtacatcaagtggccttggtatatttggcttggcttcatcgccggcctgatcgccata
gtaatggtcacaattatgctctctttatggatgtgctccaatggatcgttacaatgcag
aatttgcatttaa
B CoV S (GSAS-2P+S13+P251N+D253T) AA
SEQ ID NO: 41
MAKNVAIFGLLFSLLVLVPSQIFASQCVNLTTRTQLPPAYTNSFTRGVYYPDKVFRSSV
LHSTQDLFLPFFSNVTWFHAIHVSGINGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRSYLTNGTSSSGWTAGAAAYYVGYLQPRTELLKYNENG
TITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSFV
IRGDEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELLH
APATVCGPKKSTNLVKNKCVNFNFNGLTGTGVLTESNKKELPFQQFGRDIADTTDAVRD
PQTLEILDITPCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSASSVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPSK
RSFIEDLLFNKVTLADAGFIKQYGDCLGDIAARDLICAQKENGLTVLPPLLTDEMIAQY
TSALLAGTITSGWTFGAGAALQIPFAMQMAYRENGIGVTQNVLYENQKLIANQENSAIG
KIQDSLSSTASALGKLQDVVNQNAQALNTLVKQLSSNFGAISSVLNDILSRLDPPEAEV
QIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQRNE
YEPQIITTDNTFVSGNCDVVIGIVNNTVYDPLQPELDSFKEELDKYFKNHTSPDVDLGD
ISGINASVVNIQKEIDRLNEVAKNLNESLIDLQELGKYEQYIKWPWYIWLGFIAGLIAI
VMVTIMLSLWMCSNGSLQCRICI
B CoV S (GSAS-2P+S13+Δ247-250) DNA
SEQ ID NO: 42
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgacccggagactcctctagcggctggactgccggcgctgccgcttattacgttg
gttatcttcagccacgcacgttcctgctgaagtataacgagaatggtactattaccgat
gccgtggattgtgcccttgaccccctgtccgaaactaagtgcacactcaagtcattcac
tgtggaaaaaggaatctaccagacaagcaattttcgggtccagcctactgagagcattg
tgcgctttcctaacatcacaaatctttgccccttcggagaggttttcaatgctacacgg
tttgcctccgtgtatgcctggaaccgcaagagaatttccaattgcgtggccgattactc
cgtgctctacaatagtgcaagctttagcacctttaagtgctatggcgtatcccctacta
agcttaacgacttgtgtttcacaaacgtgtatgccgactcctttgtgatacggggcgac
gaagttagacagatagcaccaggacagacgggaaagatagctgactacaactataagct
tcctgatgacttcactggctgcgttatcgcgtggaattctaacaacctggactcaaaag
tcggcggcaactataactatctctatcggctgttccgcaagagtaaccttaagcccttt
gagagagatataagcactgaaatctaccaggctggcagtacgccctgtaatggcgtgga
aggctttaattgttattttccactgcaatcctatggttttcagccaaccaatggcgtgg
gctaccaaccataccgcgtcgtggtgctctcctttgaactgctccacgctcccgcgact
gtctgcggccccaagaagtccacgaaccttgtgaagaataagtgcgttaattttaattt
caacggcctcactggaacaggagtgctcactgagagtaacaagaagttcctgccatttc
aacaatttggcagagacatagccgatactactgacgccgttagggacccccagaccctc
gagattctcgatataacgccctgctccttcggtggagtttccgtgatcacgccaggcac
caataccagtaaccaggtcgccgtgctgtatcaggatgtcaactgtactgaggtgcccg
tagccatccatgcggatcagctcacaccaacttggagggtgtacagcaccggctccaat
gtattccagactcgggccggatgccttattggcgccgaacacgtgaacaatagttacga
atgcgatattccaattggcgccggaatctgtgctagctaccagactcagacgaactccc
caggcagcgccagcagcgttgccagccagtcaatcatcgcttatacaatgtcacttgga
gccgaaaactccgtggcttactcaaacaacagcatcgccatccccacaaacttcaccat
atccgtgacaactgagattctgccagtgtccatgactaagacgtccgtagattgcacta
tgtacatatgcggcgacagcacagaatgttctaatctgctgctgcaatatggaagcttc
tgcactcaactgaacagagcgctcacaggcatcgccgtggagcaggataagaataccca
ggaggtgttcgcccaagttaagcagatctacaagaccccacccataaaggatttcggtg
gattcaattttagtcagatactcccagacccatctaagccatccaagaggagctttatc
gaggatcttttgtttaacaaagttactctggccgacgccggtttcatcaagcagtacgg
agattgcctcggcgacatcgctgctcgtgacctcatctgtgcgcaaaagtttaacggtc
tgacggtgctgcctcccctccttactgatgaaatgatcgcccagtataccagcgcactc
ctcgctggcaccataacatccggttggacattcggcgctggtgcagcactgcagatacc
attcgccatgcaaatggcatatcgtttcaacggtatcggtgtcacacagaatgtcctat
atgagaaccagaagctgatcgcaaatcagttcaatagtgccatcggaaaaatccaggat
agccttagcagcacagcctcagcccttggcaaactccaggatgtcgtgaaccagaatgc
ccaggctctcaataccctcgtgaagcagctctcatctaatttcggcgcaatttccagtg
tcctcaacgacatcctcagccgcctcgacccccccgaggccgaagtgcagattgacaga
ctgattacaggtcgactccagagcctccagacttacgtgactcagcagctgataagagc
cgccgagataagggccagcgctaacctggctgccacaaagatgtctgagtgcgtgctgg
gccagtccaagagagtagacttctgtggcaaaggctaccatctgatgagcttcccacaa
tccgcacctcacggcgtagtgttcctccacgtgacatatgtaccggctcaggagaagaa
tttcactaccgctcctgctatatgccatgatggaaaggctcacttcccccgggaggggg
tgttcgtgtccaacggcacccattggtttgtgactcagcggaatttctacgaaccccag
atcataaccactgacaacacatttgtgtccggaaattgtgacgtggtcattggaatagt
gaacaacactgtttatgatccactgcagccagaacttgacagctttaaggaggagctcg
acaagtacttcaagaatcatacgtcaccagatgtggacctcggagatattagcggtatc
aatgccagtgttgtcaatattcagaaggaaatagaccgccttaatgaggtcgccaaaaa
tctgaacgagagcctcatcgatcttcaggagctgggcaaatatgagcagtacatcaagt
ggccttggtatatttggcttggcttcatcgccggcctgatcgccatagtaatggtcaca
attatgctctctttatggatgtgctccaatggatcgttacaatgcagaatttgcattta
a
B CoV S (GSAS-2P+S13+Δ247-250) AA
SEQ ID NO: 43
MAKNVAIFGLLFSLLVLVPSQIFASQCVNLTTRTQLPPAYTNSFTRGVYYPDKVFRSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRPGDSSSGWTAGAAAYYVGYLQPRTELLKYNENGTITD
AVDCALDPLSETKCTLKSFTVEKGIYQTSNFRVQPTESIVRFPNITNLCPFGEVENATR
FASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELLHAPAT
VCGPKKSTNLVKNKCVNFNFNGLTGTGVLTESNKKELPFQQFGRDIADTTDAVRDPQTL
EILDITPCSFGGVSVITPGTNTSNQVAVLYQDVNCTEVPVAIHADQLTPTWRVYSTGSN
VFQTRAGCLIGAEHVNNSYECDIPIGAGICASYQTQTNSPGSASSVASQSIIAYTMSLG
AENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFI
EDLLFNKVTLADAGFIKQYGDCLGDIAARDLICAQKENGLTVLPPLLTDEMIAQYTSAL
LAGTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPWYIWLGFIAGLIAIVMVT
IMLSLWMCSNGSLQCRICI
B CoV S (GSAS-2P+S13+Δ248-251) DNA
SEQ ID NO: 44
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatccggagactcctctagcggctggactgccggcgctgccgcttattacgttg
gttatcttcagccacgcacgttcctgctgaagtataacgagaatggtactattaccgat
gccgtggattgtgcccttgaccccctgtccgaaactaagtgcacactcaagtcattcac
tgtggaaaaaggaatctaccagacaagcaattttcgggtccagcctactgagagcattg
tgcgctttcctaacatcacaaatctttgccccttcggagaggttttcaatgctacacgg
tttgcctccgtgtatgcctggaaccgcaagagaatttccaattgcgtggccgattactc
cgtgctctacaatagtgcaagctttagcacctttaagtgctatggcgtatcccctacta
agcttaacgacttgtgtttcacaaacgtgtatgccgactcctttgtgatacggggcgac
gaagttagacagatagcaccaggacagacgggaaagatagctgactacaactataagct
tcctgatgacttcactggctgcgttatcgcgtggaattctaacaacctggactcaaaag
tcggcggcaactataactatctctatcggctgttccgcaagagtaaccttaagcccttt
gagagagatataagcactgaaatctaccaggctggcagtacgccctgtaatggcgtgga
aggctttaattgttattttccactgcaatcctatggttttcagccaaccaatggcgtgg
gctaccaaccataccgcgtcgtggtgctctcctttgaactgctccacgctcccgcgact
gtctgcggccccaagaagtccacgaaccttgtgaagaataagtgcgttaattttaattt
caacggcctcactggaacaggagtgctcactgagagtaacaagaagttcctgccatttc
aacaatttggcagagacatagccgatactactgacgccgttagggacccccagaccctc
gagattctcgatataacgccctgctccttcggtggagtttccgtgatcacgccaggcac
caataccagtaaccaggtcgccgtgctgtatcaggatgtcaactgtactgaggtgcccg
tagccatccatgcggatcagctcacaccaacttggagggtgtacagcaccggctccaat
gtattccagactcgggccggatgccttattggcgccgaacacgtgaacaatagttacga
atgcgatattccaattggcgccggaatctgtgctagctaccagactcagacgaactccc
caggcagcgccagcagcgttgccagccagtcaatcatcgcttatacaatgtcacttgga
gccgaaaactccgtggcttactcaaacaacagcatcgccatccccacaaacttcaccat
atccgtgacaactgagattctgccagtgtccatgactaagacgtccgtagattgcacta
tgtacatatgcggcgacagcacagaatgttctaatctgctgctgcaatatggaagcttc
tgcactcaactgaacagagcgctcacaggcatcgccgtggagcaggataagaataccca
ggaggtgttcgcccaagttaagcagatctacaagaccccacccataaaggatttcggtg
gattcaattttagtcagatactcccagacccatctaagccatccaagaggagctttatc
gaggatcttttgtttaacaaagttactctggccgacgccggtttcatcaagcagtacgg
agattgcctcggcgacatcgctgctcgtgacctcatctgtgcgcaaaagtttaacggtc
tgacggtgctgcctcccctccttactgatgaaatgatcgcccagtataccagcgcactc
ctcgctggcaccataacatccggttggacattcggcgctggtgcagcactgcagatacc
attcgccatgcaaatggcatatcgtttcaacggtatcggtgtcacacagaatgtcctat
atgagaaccagaagctgatcgcaaatcagttcaatagtgccatcggaaaaatccaggat
agccttagcagcacagcctcagcccttggcaaactccaggatgtcgtgaaccagaatgc
ccaggctctcaataccctcgtgaagcagctctcatctaatttcggcgcaatttccagtg
tcctcaacgacatcctcagccgcctcgacccccccgaggccgaagtgcagattgacaga
ctgattacaggtcgactccagagcctccagacttacgtgactcagcagctgataagagc
cgccgagataagggccagcgctaacctggctgccacaaagatgtctgagtgcgtgctgg
gccagtccaagagagtagacttctgtggcaaaggctaccatctgatgagcttcccacaa
tccgcacctcacggcgtagtgttcctccacgtgacatatgtaccggctcaggagaagaa
tttcactaccgctcctgctatatgccatgatggaaaggctcacttcccccgggaggggg
tgttcgtgtccaacggcacccattggtttgtgactcagcggaatttctacgaaccccag
atcataaccactgacaacacatttgtgtccggaaattgtgacgtggtcattggaatagt
gaacaacactgtttatgatccactgcagccagaacttgacagctttaaggaggagctcg
acaagtacttcaagaatcatacgtcaccagatgtggacctcggagatattagcggtatc
aatgccagtgttgtcaatattcagaaggaaatagaccgccttaatgaggtcgccaaaaa
tctgaacgagagcctcatcgatcttcaggagctgggcaaatatgagcagtacatcaagt
ggccttggtatatttggcttggcttcatcgccggcctgatcgccatagtaatggtcaca
attatgctctctttatggatgtgctccaatggatcgttacaatgcagaatttgcattta
a
B CoV S (GSAS-2P+S13+Δ248-251) AA
SEQ ID NO: 45
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVEKNIDGYFKIYSKHTPINLVRDLPQGFSALE
PLVDLPIGINITRFQTLLALHRSGDSSSGWTAGAAAYYVGYLQPRTFLLKYNENGTITD
AVDCALDPLSETKCTLKSFTVEKGIYQTSNERVQPTESIVRFPNITNLCPFGEVENATR
FASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELLHAPAT
VCGPKKSTNLVKNKCVNFNFNGLTGTGVLTESNKKELPFQQFGRDIADTTDAVRDPQTL
EILDITPCSFGGVSVITPGTNTSNQVAVLYQDVNCTEVPVAIHADQLTPTWRVYSTGSN
VFQTRAGCLIGAEHVNNSYECDIPIGAGICASYQTQTNSPGSASSVASQSIIAYTMSLG
AENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFI
EDLLFNKVTLADAGFIKQYGDCLGDIAARDLICAQKFNGLTVLPPLLTDEMIAQYTSAL
LAGTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPWYIWLGFIAGLIAIVMVT
IMLSLWMCSNGSLQCRICI
B CoV S (GSAS-2P+S13+Δ249-252) DNA
SEQ ID NO: 46
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatcctatgactcctctagcggctggactgccggcgctgccgcttattacgttg
gttatcttcagccacgcacgttcctgctgaagtataacgagaatggtactattaccgat
gccgtggattgtgcccttgaccccctgtccgaaactaagtgcacactcaagtcattcac
tgtggaaaaaggaatctaccagacaagcaattttcgggtccagcctactgagagcattg
tgcgctttcctaacatcacaaatctttgccccttcggagaggttttcaatgctacacgg
tttgcctccgtgtatgcctggaaccgcaagagaatttccaattgcgtggccgattactc
cgtgctctacaatagtgcaagctttagcacctttaagtgctatggcgtatcccctacta
agcttaacgacttgtgtttcacaaacgtgtatgccgactcctttgtgatacggggcgac
gaagttagacagatagcaccaggacagacgggaaagatagctgactacaactataagct
tcctgatgacttcactggctgcgttatcgcgtggaattctaacaacctggactcaaaag
tcggcggcaactataactatctctatcggctgttccgcaagagtaaccttaagcccttt
gagagagatataagcactgaaatctaccaggctggcagtacgccctgtaatggcgtgga
aggctttaattgttattttccactgcaatcctatggttttcagccaaccaatggcgtgg
gctaccaaccataccgcgtcgtggtgctctcctttgaactgctccacgctcccgcgact
gtctgcggccccaagaagtccacgaaccttgtgaagaataagtgcgttaattttaattt
caacggcctcactggaacaggagtgctcactgagagtaacaagaagttcctgccatttc
aacaatttggcagagacatagccgatactactgacgccgttagggacccccagaccctc
gagattctcgatataacgccctgctccttcggtggagtttccgtgatcacgccaggcac
caataccagtaaccaggtcgccgtgctgtatcaggatgtcaactgtactgaggtgcccg
tagccatccatgcggatcagctcacaccaacttggagggtgtacagcaccggctccaat
gtattccagactcgggccggatgccttattggcgccgaacacgtgaacaatagttacga
atgcgatattccaattggcgccggaatctgtgctagctaccagactcagacgaactccc
caggcagcgccagcagcgttgccagccagtcaatcatcgcttatacaatgtcacttgga
gccgaaaactccgtggcttactcaaacaacagcatcgccatccccacaaacttcaccat
atccgtgacaactgagattctgccagtgtccatgactaagacgtccgtagattgcacta
tgtacatatgcggcgacagcacagaatgttctaatctgctgctgcaatatggaagcttc
tgcactcaactgaacagagcgctcacaggcatcgccgtggagcaggataagaataccca
ggaggtgttcgcccaagttaagcagatctacaagaccccacccataaaggatttcggtg
gattcaattttagtcagatactcccagacccatctaagccatccaagaggagctttatc
gaggatcttttgtttaacaaagttactctggccgacgccggtttcatcaagcagtacgg
agattgcctcggcgacatcgctgctcgtgacctcatctgtgcgcaaaagtttaacggtc
tgacggtgctgcctcccctccttactgatgaaatgatcgcccagtataccagcgcactc
ctcgctggcaccataacatccggttggacattcggcgctggtgcagcactgcagatacc
attcgccatgcaaatggcatatcgtttcaacggtatcggtgtcacacagaatgtcctat
atgagaaccagaagctgatcgcaaatcagttcaatagtgccatcggaaaaatccaggat
agccttagcagcacagcctcagcccttggcaaactccaggatgtcgtgaaccagaatgc
ccaggctctcaataccctcgtgaagcagctctcatctaatttcggcgcaatttccagtg
tcctcaacgacatcctcagccgcctcgacccccccgaggccgaagtgcagattgacaga
ctgattacaggtcgactccagagcctccagacttacgtgactcagcagctgataagagc
cgccgagataagggccagcgctaacctggctgccacaaagatgtctgagtgcgtgctgg
gccagtccaagagagtagacttctgtggcaaaggctaccatctgatgagcttcccacaa
tccgcacctcacggcgtagtgttcctccacgtgacatatgtaccggctcaggagaagaa
tttcactaccgctcctgctatatgccatgatggaaaggctcacttcccccgggaggggg
tgttcgtgtccaacggcacccattggtttgtgactcagcggaatttctacgaaccccag
atcataaccactgacaacacatttgtgtccggaaattgtgacgtggtcattggaatagt
gaacaacactgtttatgatccactgcagccagaacttgacagctttaaggaggagctcg
acaagtacttcaagaatcatacgtcaccagatgtggacctcggagatattagcggtatc
aatgccagtgttgtcaatattcagaaggaaatagaccgccttaatgaggtcgccaaaaa
tctgaacgagagcctcatcgatcttcaggagctgggcaaatatgagcagtacatcaagt
ggccttggtatatttggcttggcttcatcgccggcctgatcgccatagtaatggtcaca
attatgctctctttatggatgtgctccaatggatcgttacaatgcagaatttgcattta
a
B CoV S (GSAS-2P+S13+Δ249-252) AA
SEQ ID NO: 47
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIE
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRSYDSSSGWTAGAAAYYVGYLQPRTELLKYNENGTITD
AVDCALDPLSETKCTLKSFTVEKGIYQTSNERVQPTESIVRFPNITNLCPFGEVENATR
FASVYAWNRKRISNCVADYSVLYNSASFSTFKCYGVSPTKLNDLCFTNVYADSFVIRGD
EVRQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVLSFELLHAPAT
VCGPKKSTNLVKNKCVNFNFNGLTGTGVLTESNKKELPFQQFGRDIADTTDAVRDPQTL
EILDITPCSFGGVSVITPGTNTSNQVAVLYQDVNCTEVPVAIHADQLTPTWRVYSTGSN
VFQTRAGCLIGAEHVNNSYECDIPIGAGICASYQTQTNSPGSASSVASQSIIAYTMSLG
AENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFI
EDLLFNKVTLADAGFIKQYGDCLGDIAARDLICAQKENGLTVLPPLLTDEMIAQYTSAL
LAGTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDPPEAEVQIDR
LITGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPWYIWLGFIAGLIAIVMVT
IMLSLWMCSNGSLQCRICI
B CoV S (GSAS-2P+S13+Δ246-250) DNA
SEQ ID NO: 48
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccccggagactcctctagcggctggactgccggcgctgccgcttattacgttggtt
atcttcagccacgcacgttcctgctgaagtataacgagaatggtactattaccgatgcc
gtggattgtgcccttgaccccctgtccgaaactaagtgcacactcaagtcattcactgt
ggaaaaaggaatctaccagacaagcaattttcgggtccagcctactgagagcattgtgc
gctttcctaacatcacaaatctttgccccttcggagaggttttcaatgctacacggttt
gcctccgtgtatgcctggaaccgcaagagaatttccaattgcgtggccgattactccgt
gctctacaatagtgcaagctttagcacctttaagtgctatggcgtatcccctactaagc
ttaacgacttgtgtttcacaaacgtgtatgccgactcctttgtgatacggggcgacgaa
gttagacagatagcaccaggacagacgggaaagatagctgactacaactataagcttcc
tgatgacttcactggctgcgttatcgcgtggaattctaacaacctggactcaaaagtcg
gcggcaactataactatctctatcggctgttccgcaagagtaaccttaagccctttgag
agagatataagcactgaaatctaccaggctggcagtacgccctgtaatggcgtggaagg
ctttaattgttattttccactgcaatcctatggttttcagccaaccaatggcgtgggct
accaaccataccgcgtcgtggtgctctcctttgaactgctccacgctcccgcgactgtc
tgcggccccaagaagtccacgaaccttgtgaagaataagtgcgttaattttaatttcaa
cggcctcactggaacaggagtgctcactgagagtaacaagaagttcctgccatttcaac
aatttggcagagacatagccgatactactgacgccgttagggacccccagaccctcgag
attctcgatataacgccctgctccttcggtggagtttccgtgatcacgccaggcaccaa
taccagtaaccaggtcgccgtgctgtatcaggatgtcaactgtactgaggtgcccgtag
ccatccatgcggatcagctcacaccaacttggagggtgtacagcaccggctccaatgta
ttccagactcgggccggatgccttattggcgccgaacacgtgaacaatagttacgaatg
cgatattccaattggcgccggaatctgtgctagctaccagactcagacgaactccccag
gcagcgccagcagcgttgccagccagtcaatcatcgcttatacaatgtcacttggagcc
gaaaactccgtggcttactcaaacaacagcatcgccatccccacaaacttcaccatatc
cgtgacaactgagattctgccagtgtccatgactaagacgtccgtagattgcactatgt
acatatgcggcgacagcacagaatgttctaatctgctgctgcaatatggaagcttctgc
actcaactgaacagagcgctcacaggcatcgccgtggagcaggataagaatacccagga
ggtgttcgcccaagttaagcagatctacaagaccccacccataaaggatttcggtggat
tcaattttagtcagatactcccagacccatctaagccatccaagaggagctttatcgag
gatcttttgtttaacaaagttactctggccgacgccggtttcatcaagcagtacggaga
ttgcctcggcgacatcgctgctcgtgacctcatctgtgcgcaaaagtttaacggtctga
cggtgctgcctcccctccttactgatgaaatgatcgcccagtataccagcgcactcctc
gctggcaccataacatccggttggacattcggcgctggtgcagcactgcagataccatt
cgccatgcaaatggcatatcgtttcaacggtatcggtgtcacacagaatgtcctatatg
agaaccagaagctgatcgcaaatcagttcaatagtgccatcggaaaaatccaggatagc
cttagcagcacagcctcagcccttggcaaactccaggatgtcgtgaaccagaatgccca
ggctctcaataccctcgtgaagcagctctcatctaatttcggcgcaatttccagtgtcc
tcaacgacatcctcagccgcctcgacccccccgaggccgaagtgcagattgacagactg
attacaggtcgactccagagcctccagacttacgtgactcagcagctgataagagccgc
cgagataagggccagcgctaacctggctgccacaaagatgtctgagtgcgtgctgggcc
agtccaagagagtagacttctgtggcaaaggctaccatctgatgagcttcccacaatcc
gcacctcacggcgtagtgttcctccacgtgacatatgtaccggctcaggagaagaattt
cactaccgctcctgctatatgccatgatggaaaggctcacttcccccgggagggggtgt
tcgtgtccaacggcacccattggtttgtgactcagcggaatttctacgaaccccagatc
ataaccactgacaacacatttgtgtccggaaattgtgacgtggtcattggaatagtgaa
caacactgtttatgatccactgcagccagaacttgacagctttaaggaggagctcgaca
agtacttcaagaatcatacgtcaccagatgtggacctcggagatattagcggtatcaat
gccagtgttgtcaatattcagaaggaaatagaccgccttaatgaggtcgccaaaaatct
gaacgagagcctcatcgatcttcaggagctgggcaaatatgagcagtacatcaagtggc
cttggtatatttggcttggcttcatcgccggcctgatcgccatagtaatggtcacaatt
atgctctctttatggatgtgctccaatggatcgttacaatgcagaatttgcatttaa
B CoV S (GSAS-2P+S13+Δ246-250) AA
SEQ ID NO: 49
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVEKNIDGYFKIYSKHTPINLVRDLPQGFSALE
PLVDLPIGINITRFQTLLALHPGDSSSGWTAGAAAYYVGYLQPRTFLLKYNENGTITDA
VDCALDPLSETKCTLKSFTVEKGIYQTSNERVQPTESIVRFPNITNLCPFGEVENATRF
ASVYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLSFELLHAPATV
CGPKKSTNLVKNKCVNFNFNGLTGTGVLTESNKKELPFQQFGRDIADTTDAVRDPQTLE
ILDITPCSFGGVSVITPGTNTSNQVAVLYQDVNCTEVPVAIHADQLTPTWRVYSTGSNV
FQTRAGCLIGAEHVNNSYECDIPIGAGICASYQTQTNSPGSASSVASQSIIAYTMSLGA
ENSVAYSNNSIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPSKPSKRSFIE
DLLFNKVTLADAGFIKQYGDCLGDIAARDLICAQKENGLTVLPPLLTDEMIAQYTSALL
AGTITSGWTFGAGAALQIPFAMQMAYRENGIGVTQNVLYENQKLIANQFNSAIGKIQDS
LSSTASALGKLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGLIAIVMVTI
MLSLWMCSNGSLQCRICI*
B CoV S (GSAS-2P+S13+Δ247-251) DNA
SEQ ID NO: 50
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgaggagactcctctagcggctggactgccggcgctgccgcttattacgttggtt
atcttcagccacgcacgttcctgctgaagtataacgagaatggtactattaccgatgcc
gtggattgtgcccttgaccccctgtccgaaactaagtgcacactcaagtcattcactgt
ggaaaaaggaatctaccagacaagcaattttcgggtccagcctactgagagcattgtgc
gctttcctaacatcacaaatctttgccccttcggagaggttttcaatgctacacggttt
gcctccgtgtatgcctggaaccgcaagagaatttccaattgcgtggccgattactccgt
gctctacaatagtgcaagctttagcacctttaagtgctatggcgtatcccctactaagc
ttaacgacttgtgtttcacaaacgtgtatgccgactcctttgtgatacggggcgacgaa
gttagacagatagcaccaggacagacgggaaagatagctgactacaactataagcttcc
tgatgacttcactggctgcgttatcgcgtggaattctaacaacctggactcaaaagtcg
gcggcaactataactatctctatcggctgttccgcaagagtaaccttaagccctttgag
agagatataagcactgaaatctaccaggctggcagtacgccctgtaatggcgtggaagg
ctttaattgttattttccactgcaatcctatggttttcagccaaccaatggcgtgggct
accaaccataccgcgtcgtggtgctctcctttgaactgctccacgctcccgcgactgtc
tgcggccccaagaagtccacgaaccttgtgaagaataagtgcgttaattttaatttcaa
cggcctcactggaacaggagtgctcactgagagtaacaagaagttcctgccatttcaac
aatttggcagagacatagccgatactactgacgccgttagggacccccagaccctcgag
attctcgatataacgccctgctccttcggtggagtttccgtgatcacgccaggcaccaa
taccagtaaccaggtcgccgtgctgtatcaggatgtcaactgtactgaggtgcccgtag
ccatccatgcggatcagctcacaccaacttggagggtgtacagcaccggctccaatgta
ttccagactcgggccggatgccttattggcgccgaacacgtgaacaatagttacgaatg
cgatattccaattggcgccggaatctgtgctagctaccagactcagacgaactccccag
gcagcgccagcagcgttgccagccagtcaatcatcgcttatacaatgtcacttggagcc
gaaaactccgtggcttactcaaacaacagcatcgccatccccacaaacttcaccatatc
cgtgacaactgagattctgccagtgtccatgactaagacgtccgtagattgcactatgt
acatatgcggcgacagcacagaatgttctaatctgctgctgcaatatggaagcttctgc
actcaactgaacagagcgctcacaggcatcgccgtggagcaggataagaatacccagga
ggtgttcgcccaagttaagcagatctacaagaccccacccataaaggatttcggtggat
tcaattttagtcagatactcccagacccatctaagccatccaagaggagctttatcgag
gatcttttgtttaacaaagttactctggccgacgccggtttcatcaagcagtacggaga
ttgcctcggcgacatcgctgctcgtgacctcatctgtgcgcaaaagtttaacggtctga
cggtgctgcctcccctccttactgatgaaatgatcgcccagtataccagcgcactcctc
gctggcaccataacatccggttggacattcggcgctggtgcagcactgcagataccatt
cgccatgcaaatggcatatcgtttcaacggtatcggtgtcacacagaatgtcctatatg
agaaccagaagctgatcgcaaatcagttcaatagtgccatcggaaaaatccaggatagc
cttagcagcacagcctcagcccttggcaaactccaggatgtcgtgaaccagaatgccca
ggctctcaataccctcgtgaagcagctctcatctaatttcggcgcaatttccagtgtcc
tcaacgacatcctcagccgcctcgacccccccgaggccgaagtgcagattgacagactg
attacaggtcgactccagagcctccagacttacgtgactcagcagctgataagagccgc
cgagataagggccagcgctaacctggctgccacaaagatgtctgagtgcgtgctgggcc
agtccaagagagtagacttctgtggcaaaggctaccatctgatgagcttcccacaatcc
gcacctcacggcgtagtgttcctccacgtgacatatgtaccggctcaggagaagaattt
cactaccgctcctgctatatgccatgatggaaaggctcacttcccccgggagggggtgt
tcgtgtccaacggcacccattggtttgtgactcagcggaatttctacgaaccccagatc
ataaccactgacaacacatttgtgtccggaaattgtgacgtggtcattggaatagtgaa
caacactgtttatgatccactgcagccagaacttgacagctttaaggaggagctcgaca
agtacttcaagaatcatacgtcaccagatgtggacctcggagatattagcggtatcaat
gccagtgttgtcaatattcagaaggaaatagaccgccttaatgaggtcgccaaaaatct
gaacgagagcctcatcgatcttcaggagctgggcaaatatgagcagtacatcaagtggc
cttggtatatttggcttggcttcatcgccggcctgatcgccatagtaatggtcacaatt
atgctctctttatggatgtgctccaatggatcgttacaatgcagaatttgcatttaa
B CoV S (GSAS-2P+S13+Δ247-251) AA
SEQ ID NO: 51
MAKNVAIFGLLFSLLVLVPSQIFASQCVNLTTRTQLPPAYTNSFTRGVYYPDKVFRSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRGDSSSGWTAGAAAYYVGYLQPRTFLLKYNENGTITDA
VDCALDPLSETKCTLKSFTVEKGIYQTSNERVQPTESIVRFPNITNLCPFGEVENATRE
ASVYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLSFELLHAPATV
CGPKKSTNLVKNKCVNFNFNGLTGTGVLTESNKKFLPFQQFGRDIADTTDAVRDPQTLE
ILDITPCSFGGVSVITPGTNTSNQVAVLYQDVNCTEVPVAIHADQLTPTWRVYSTGSNV
FQTRAGCLIGAEHVNNSYECDIPIGAGICASYQTQTNSPGSASSVASQSIIAYTMSLGA
ENSVAYSNNSIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPSKPSKRSFIE
DLLFNKVTLADAGFIKQYGDCLGDIAARDLICAQKENGLTVLPPLLTDEMIAQYTSALL
AGTITSGWTFGAGAALQIPFAMQMAYRENGIGVTQNVLYENQKLIANQFNSAIGKIQDS
LSSTASALGKLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGLIAIVMVTI
MLSLWMCSNGSLQCRICI
B CoV S (GSAS-2P+S13+Δ248-252) DNA
SEQ ID NO: 52
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgatccgactcctctagcggctggactgccggcgctgccgcttattacgttggtt
atcttcagccacgcacgttcctgctgaagtataacgagaatggtactattaccgatgcc
gtggattgtgcccttgaccccctgtccgaaactaagtgcacactcaagtcattcactgt
ggaaaaaggaatctaccagacaagcaattttcgggtccagcctactgagagcattgtgc
gctttcctaacatcacaaatctttgccccttcggagaggttttcaatgctacacggttt
gcctccgtgtatgcctggaaccgcaagagaatttccaattgcgtggccgattactccgt
gctctacaatagtgcaagctttagcacctttaagtgctatggcgtatcccctactaagc
ttaacgacttgtgtttcacaaacgtgtatgccgactcctttgtgatacggggcgacgaa
gttagacagatagcaccaggacagacgggaaagatagctgactacaactataagcttcc
tgatgacttcactggctgcgttatcgcgtggaattctaacaacctggactcaaaagtcg
gcggcaactataactatctctatcggctgttccgcaagagtaaccttaagccctttgag
agagatataagcactgaaatctaccaggctggcagtacgccctgtaatggcgtggaagg
ctttaattgttattttccactgcaatcctatggttttcagccaaccaatggcgtgggct
accaaccataccgcgtcgtggtgctctcctttgaactgctccacgctcccgcgactgtc
tgcggccccaagaagtccacgaaccttgtgaagaataagtgcgttaattttaatttcaa
cggcctcactggaacaggagtgctcactgagagtaacaagaagttcctgccatttcaac
aatttggcagagacatagccgatactactgacgccgttagggacccccagaccctcgag
attctcgatataacgccctgctccttcggtggagtttccgtgatcacgccaggcaccaa
taccagtaaccaggtcgccgtgctgtatcaggatgtcaactgtactgaggtgcccgtag
ccatccatgcggatcagctcacaccaacttggagggtgtacagcaccggctccaatgta
ttccagactcgggccggatgccttattggcgccgaacacgtgaacaatagttacgaatg
cgatattccaattggcgccggaatctgtgctagctaccagactcagacgaactccccag
gcagcgccagcagcgttgccagccagtcaatcatcgcttatacaatgtcacttggagcc
gaaaactccgtggcttactcaaacaacagcatcgccatccccacaaacttcaccatatc
cgtgacaactgagattctgccagtgtccatgactaagacgtccgtagattgcactatgt
acatatgcggcgacagcacagaatgttctaatctgctgctgcaatatggaagcttctgc
actcaactgaacagagcgctcacaggcatcgccgtggagcaggataagaatacccagga
ggtgttcgcccaagttaagcagatctacaagaccccacccataaaggatttcggtggat
tcaattttagtcagatactcccagacccatctaagccatccaagaggagctttatcgag
gatcttttgtttaacaaagttactctggccgacgccggtttcatcaagcagtacggaga
ttgcctcggcgacatcgctgctcgtgacctcatctgtgcgcaaaagtttaacggtctga
cggtgctgcctcccctccttactgatgaaatgatcgcccagtataccagcgcactcctc
gctggcaccataacatccggttggacattcggcgctggtgcagcactgcagataccatt
cgccatgcaaatggcatatcgtttcaacggtatcggtgtcacacagaatgtcctatatg
agaaccagaagctgatcgcaaatcagttcaatagtgccatcggaaaaatccaggatagc
cttagcagcacagcctcagcccttggcaaactccaggatgtcgtgaaccagaatgccca
ggctctcaataccctcgtgaagcagctctcatctaatttcggcgcaatttccagtgtcc
tcaacgacatcctcagccgcctcgacccccccgaggccgaagtgcagattgacagactg
attacaggtcgactccagagcctccagacttacgtgactcagcagctgataagagccgc
cgagataagggccagcgctaacctggctgccacaaagatgtctgagtgcgtgctgggcc
agtccaagagagtagacttctgtggcaaaggctaccatctgatgagcttcccacaatcc
gcacctcacggcgtagtgttcctccacgtgacatatgtaccggctcaggagaagaattt
cactaccgctcctgctatatgccatgatggaaaggctcacttcccccgggagggggtgt
tcgtgtccaacggcacccattggtttgtgactcagcggaatttctacgaaccccagatc
ataaccactgacaacacatttgtgtccggaaattgtgacgtggtcattggaatagtgaa
caacactgtttatgatccactgcagccagaacttgacagctttaaggaggagctcgaca
agtacttcaagaatcatacgtcaccagatgtggacctcggagatattagcggtatcaat
gccagtgttgtcaatattcagaaggaaatagaccgccttaatgaggtcgccaaaaatct
gaacgagagcctcatcgatcttcaggagctgggcaaatatgagcagtacatcaagtggc
cttggtatatttggcttggcttcatcgccggcctgatcgccatagtaatggtcacaatt
atgctctctttatggatgtgctccaatggatcgttacaatgcagaatttgcatttaa
B CoV S (GSAS-2P+S13+Δ248-252) AA
SEQ ID NO: 53
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHRSDSSSGWTAGAAAYYVGYLQPRTFLLKYNENGTITDA
VDCALDPLSETKCTLKSFTVEKGIYQTSNERVQPTESIVRFPNITNLCPFGEVFNATRF
ASVYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYLYRLFRKSNLKPFE
RDISTEIYQAGSTPCNGVEGENCYFPLQSYGFQPTNGVGYQPYRVVVLSFELLHAPATV
CGPKKSTNLVKNKCVNFNFNGLTGTGVLTESNKKELPFQQFGRDIADTTDAVRDPQTLE
ILDITPCSFGGVSVITPGTNTSNQVAVLYQDVNCTEVPVAIHADQLTPTWRVYSTGSNV
FQTRAGCLIGAEHVNNSYECDIPIGAGICASYQTQTNSPGSASSVASQSIIAYTMSLGA
ENSVAYSNNSIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPSKPSKRSFIE
DLLFNKVTLADAGFIKQYGDCLGDIAARDLICAQKENGLTVLPPLLTDEMIAQYTSALL
AGTITSGWTFGAGAALQIPFAMQMAYRENGIGVTQNVLYENQKLIANQFNSAIGKIQDS
LSSTASALGKLQDVVNQNAQALNTLVKQLSSNFGAISSVLNDILSRLDPPEAEVQIDRL
ITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQI
ITTDNTFVSGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGLIAIVMVTI
MLSLWMCSNGSLQCRICI
B CoV S (GSAS-2P+S13+Δ246-251) DNA
SEQ ID NO: 54
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccacggagactcctctagcggctggactgccggcgctgccgcttattacgttggttatc
ttcagccacgcacgttcctgctgaagtataacgagaatggtactattaccgatgccgtg
gattgtgcccttgaccccctgtccgaaactaagtgcacactcaagtcattcactgtgga
aaaaggaatctaccagacaagcaattttcgggtccagcctactgagagcattgtgcgct
ttcctaacatcacaaatctttgccccttcggagaggttttcaatgctacacggtttgcc
tccgtgtatgcctggaaccgcaagagaatttccaattgcgtggccgattactccgtgct
ctacaatagtgcaagctttagcacctttaagtgctatggcgtatcccctactaagctta
acgacttgtgtttcacaaacgtgtatgccgactcctttgtgatacggggcgacgaagtt
agacagatagcaccaggacagacgggaaagatagctgactacaactataagcttcctga
tgacttcactggctgcgttatcgcgtggaattctaacaacctggactcaaaagtcggcg
gcaactataactatctctatcggctgttccgcaagagtaaccttaagccctttgagaga
gatataagcactgaaatctaccaggctggcagtacgccctgtaatggcgtggaaggctt
taattgttattttccactgcaatcctatggttttcagccaaccaatggcgtgggctacc
aaccataccgcgtcgtggtgctctcctttgaactgctccacgctcccgcgactgtctgc
ggccccaagaagtccacgaaccttgtgaagaataagtgcgttaattttaatttcaacgg
cctcactggaacaggagtgctcactgagagtaacaagaagttcctgccatttcaacaat
ttggcagagacatagccgatactactgacgccgttagggacccccagaccctcgagatt
ctcgatataacgccctgctccttcggtggagtttccgtgatcacgccaggcaccaatac
cagtaaccaggtcgccgtgctgtatcaggatgtcaactgtactgaggtgcccgtagcca
tccatgcggatcagctcacaccaacttggagggtgtacagcaccggctccaatgtattc
cagactcgggccggatgccttattggcgccgaacacgtgaacaatagttacgaatgcga
tattccaattggcgccggaatctgtgctagctaccagactcagacgaactccccaggca
gcgccagcagcgttgccagccagtcaatcatcgcttatacaatgtcacttggagccgaa
aactccgtggcttactcaaacaacagcatcgccatccccacaaacttcaccatatccgt
gacaactgagattctgccagtgtccatgactaagacgtccgtagattgcactatgtaca
tatgcggcgacagcacagaatgttctaatctgctgctgcaatatggaagcttctgcact
caactgaacagagcgctcacaggcatcgccgtggagcaggataagaatacccaggaggt
gttcgcccaagttaagcagatctacaagaccccacccataaaggatttcggtggattca
attttagtcagatactcccagacccatctaagccatccaagaggagctttatcgaggat
cttttgtttaacaaagttactctggccgacgccggtttcatcaagcagtacggagattg
cctcggcgacatcgctgctcgtgacctcatctgtgcgcaaaagtttaacggtctgacgg
tgctgcctcccctccttactgatgaaatgatcgcccagtataccagcgcactcctcgct
ggcaccataacatccggttggacattcggcgctggtgcagcactgcagataccattcgc
catgcaaatggcatatcgtttcaacggtatcggtgtcacacagaatgtcctatatgaga
accagaagctgatcgcaaatcagttcaatagtgccatcggaaaaatccaggatagcctt
agcagcacagcctcagcccttggcaaactccaggatgtcgtgaaccagaatgcccaggc
tctcaataccctcgtgaagcagctctcatctaatttcggcgcaatttccagtgtcctca
acgacatcctcagccgcctcgacccccccgaggccgaagtgcagattgacagactgatt
acaggtcgactccagagcctccagacttacgtgactcagcagctgataagagccgccga
gataagggccagcgctaacctggctgccacaaagatgtctgagtgcgtgctgggccagt
ccaagagagtagacttctgtggcaaaggctaccatctgatgagcttcccacaatccgca
cctcacggcgtagtgttcctccacgtgacatatgtaccggctcaggagaagaatttcac
taccgctcctgctatatgccatgatggaaaggctcacttcccccgggagggggtgttcg
tgtccaacggcacccattggtttgtgactcagcggaatttctacgaaccccagatcata
accactgacaacacatttgtgtccggaaattgtgacgtggtcattggaatagtgaacaa
cactgtttatgatccactgcagccagaacttgacagctttaaggaggagctcgacaagt
acttcaagaatcatacgtcaccagatgtggacctcggagatattagcggtatcaatgcc
agtgttgtcaatattcagaaggaaatagaccgccttaatgaggtcgccaaaaatctgaa
cgagagcctcatcgatcttcaggagctgggcaaatatgagcagtacatcaagtggcctt
ggtatatttggcttggcttcatcgccggcctgatcgccatagtaatggtcacaattatg
ctctctttatggatgtgctccaatggatcgttacaatgcagaatttgcatttaa
B CoV S (GSAS-2P+S13+Δ246-251) AA
SEQ ID NO: 55
MAKNVAIFGLLFSLLVLVPSQIFASQCVNLTTRTQLPPAYTNSFTRGVYYPDKVERSSV
LHSTQDLFLPFFSNVTWFHAIHVSGTNGTKRFDNPVLPFNDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGFSALE
PLVDLPIGINITRFQTLLALHGDSSSGWTAGAAAYYVGYLQPRTELLKYNENGTITDAV
DCALDPLSETKCTLKSFTVEKGIYQTSNERVQPTESIVRFPNITNLCPFGEVENATRFA
SVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSFELLHAPATVC
GPKKSTNLVKNKCVNFNFNGLTGTGVLTESNKKFLPFQQFGRDIADTTDAVRDPQTLEI
LDITPCSFGGVSVITPGTNTSNQVAVLYQDVNCTEVPVAIHADQLTPTWRVYSTGSNVF
QTRAGCLIGAEHVNNSYECDIPIGAGICASYQTQTNSPGSASSVASQSIIAYTMSLGAE
NSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIED
LLENKVTLADAGFIKQYGDCLGDIAARDLICAQKENGLTVLPPLLTDEMIAQYTSALLA
GTITSGWTFGAGAALQIPFAMQMAYRENGIGVTQNVLYENQKLIANQFNSAIGKIQDSL
SSTASALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSA
PHGVVFLHVTYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWPWYIWLGFIAGLIAIVMVTIM
LSLWMCSNGSLQCRICI*
B CoV S (GSAS-2P+S13+Δ247-252) DNA
SEQ ID NO: 56
atggcgaaaaacgttgcgattttcggcttattgttttctcttcttgtgttggttccttc
tcagatcttcgcgTCCCAATGCgtgaatcttacgacgcgaacacagttaccacccgcat
atacaaatagcttcactcggggtgtttattaccccgacaaagtgttcaggtcctccgtg
ctccactcaacacaggacctctttcttcctttcttttctaacgtgacatggtttcatgc
cattcatgtatccggcactaacggtactaagaggttcgataatcctgtgctccctttca
atgacggcgtttactttgcaagcacagagaagagtaacatcatccgaggttggatcttt
ggcactaccctcgattcaaagacgcagagcctcctcattgtgaacaatgccactaacgt
ggtgatcaaagtttgcgagtttcagttctgcaatgaccctttcttgggggtgtactatc
ataagaacaacaagtcttggatggaatctgaattccgcgtctatagcagcgccaacaac
tgcacctttgaatacgtgtcccagcccttccttatggacctggagggaaagcagggaaa
ctttaagaatctgagagagttcgtgtttaaaaatatcgacggctattttaagatctatt
ctaagcacacgcctattaatctcgtgcgcgatcttccacaaggcttcagcgccctggaa
ccactcgtggacctcccaattggtatcaacatcactagatttcagactctgcttgccct
ccaccgagactcctctagcggctggactgccggcgctgccgcttattacgttggttatc
ttcagccacgcacgttcctgctgaagtataacgagaatggtactattaccgatgccgtg
gattgtgcccttgaccccctgtccgaaactaagtgcacactcaagtcattcactgtgga
aaaaggaatctaccagacaagcaattttcgggtccagcctactgagagcattgtgcgct
ttcctaacatcacaaatctttgccccttcggagaggttttcaatgctacacggtttgcc
tccgtgtatgcctggaaccgcaagagaatttccaattgcgtggccgattactccgtgct
ctacaatagtgcaagctttagcacctttaagtgctatggcgtatcccctactaagctta
acgacttgtgtttcacaaacgtgtatgccgactcctttgtgatacggggcgacgaagtt
agacagatagcaccaggacagacgggaaagatagctgactacaactataagcttcctga
tgacttcactggctgcgttatcgcgtggaattctaacaacctggactcaaaagtcggcg
gcaactataactatctctatcggctgttccgcaagagtaaccttaagccctttgagaga
gatataagcactgaaatctaccaggctggcagtacgccctgtaatggcgtggaaggctt
taattgttattttccactgcaatcctatggttttcagccaaccaatggcgtgggctacc
aaccataccgcgtcgtggtgctctcctttgaactgctccacgctcccgcgactgtctgc
ggccccaagaagtccacgaaccttgtgaagaataagtgcgttaattttaatttcaacgg
cctcactggaacaggagtgctcactgagagtaacaagaagttcctgccatttcaacaat
ttggcagagacatagccgatactactgacgccgttagggacccccagaccctcgagatt
ctcgatataacgccctgctccttcggtggagtttccgtgatcacgccaggcaccaatac
cagtaaccaggtcgccgtgctgtatcaggatgtcaactgtactgaggtgcccgtagcca
tccatgcggatcagctcacaccaacttggagggtgtacagcaccggctccaatgtattc
cagactcgggccggatgccttattggcgccgaacacgtgaacaatagttacgaatgcga
tattccaattggcgccggaatctgtgctagctaccagactcagacgaactccccaggca
gcgccagcagcgttgccagccagtcaatcatcgcttatacaatgtcacttggagccgaa
aactccgtggcttactcaaacaacagcatcgccatccccacaaacttcaccatatccgt
gacaactgagattctgccagtgtccatgactaagacgtccgtagattgcactatgtaca
tatgcggcgacagcacagaatgttctaatctgctgctgcaatatggaagcttctgcact
caactgaacagagcgctcacaggcatcgccgtggagcaggataagaatacccaggaggt
gttcgcccaagttaagcagatctacaagaccccacccataaaggatttcggtggattca
attttagtcagatactcccagacccatctaagccatccaagaggagctttatcgaggat
cttttgtttaacaaagttactctggccgacgccggtttcatcaagcagtacggagattg
cctcggcgacatcgctgctcgtgacctcatctgtgcgcaaaagtttaacggtctgacgg
tgctgcctcccctccttactgatgaaatgatcgcccagtataccagcgcactcctcgct
ggcaccataacatccggttggacattcggcgctggtgcagcactgcagataccattcgc
catgcaaatggcatatcgtttcaacggtatcggtgtcacacagaatgtcctatatgaga
accagaagctgatcgcaaatcagttcaatagtgccatcggaaaaatccaggatagcctt
agcagcacagcctcagcccttggcaaactccaggatgtcgtgaaccagaatgcccaggc
tctcaataccctcgtgaagcagctctcatctaatttcggcgcaatttccagtgtcctca
acgacatcctcagccgcctcgacccccccgaggccgaagtgcagattgacagactgatt
acaggtcgactccagagcctccagacttacgtgactcagcagctgataagagccgccga
gataagggccagcgctaacctggctgccacaaagatgtctgagtgcgtgctgggccagt
ccaagagagtagacttctgtggcaaaggctaccatctgatgagcttcccacaatccgca
cctcacggcgtagtgttcctccacgtgacatatgtaccggctcaggagaagaatttcac
taccgctcctgctatatgccatgatggaaaggctcacttcccccgggagggggtgttcg
tgtccaacggcacccattggtttgtgactcagcggaatttctacgaaccccagatcata
accactgacaacacatttgtgtccggaaattgtgacgtggtcattggaatagtgaacaa
cactgtttatgatccactgcagccagaacttgacagctttaaggaggagctcgacaagt
acttcaagaatcatacgtcaccagatgtggacctcggagatattagcggtatcaatgcc
agtgttgtcaatattcagaaggaaatagaccgccttaatgaggtcgccaaaaatctgaa
cgagagcctcatcgatcttcaggagctgggcaaatatgagcagtacatcaagtggcctt
ggtatatttggcttggcttcatcgccggcctgatcgccatagtaatggtcacaattatg
ctctctttatggatgtgctccaatggatcgttacaatgcagaatttgcatttaa
B CoV S (GSAS-2P+S13+Δ247-252) AA
SEQ ID NO: 57
MAKNVAIFGLLFSLLVLVPSQIFASQCVNLTTRTQLPPAYTNSFTRGVYYPDKVFRSSV
LHSTQDLFLPFFSNVTWFHAIHVSGTNGTKRFDNPVLPENDGVYFASTEKSNIIRGWIF
GTTLDSKTQSLLIVNNATNVVIKVCEFQFCNDPFLGVYYHKNNKSWMESEFRVYSSANN
CTFEYVSQPFLMDLEGKQGNFKNLREFVFKNIDGYFKIYSKHTPINLVRDLPQGESALE
PLVDLPIGINITRFQTLLALHRDSSSGWTAGAAAYYVGYLQPRTELLKYNENGTITDAV
DCALDPLSETKCTLKSFTVEKGIYQTSNERVQPTESIVRFPNITNLCPFGEVENATRFA
SVYAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSFELLHAPATVC
GPKKSTNLVKNKCVNFNFNGLTGTGVLTESNKKELPFQQFGRDIADTTDAVRDPQTLEI
LDITPCSFGGVSVITPGTNTSNQVAVLYQDVNCTEVPVAIHADQLTPTWRVYSTGSNVF
QTRAGCLIGAEHVNNSYECDIPIGAGICASYQTQTNSPGSASSVASQSIIAYTMSLGAE
NSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIED
LLFNKVTLADAGFIKQYGDCLGDIAARDLICAQKENGLTVLPPLLTDEMIAQYTSALLA
GTITSGWTFGAGAALQIPFAMQMAYRENGIGVTQNVLYENQKLIANQFNSAIGKIQDSL
SSTASALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSA
PHGVVFLHVTYVPAQEKNFTTAPAICHDGKAHFPREGVFVSNGTHWFVTQRNFYEPQII
TTDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWPWYIWLGFIAGLIAIVMVTIM
LSLWMCSNGSLQCRICI*

1. A modified coronavirus S protein, the modified coronavirus S protein comprising one or more than one amino acid sequence modification when compared to a corresponding parent amino acid sequence, wherein the one or more than one modification stabilize the modified coronavirus S protein and wherein the one or more than one modification comprises:
i) a substitution of one or more than one amino acid to introduce a N-glycosylation site at a position corresponding to positions 251, 252 or 253 of reference sequence SEQ ID NO: 1, wherein the N-glycosylation site is asparagine (N) in the consensus sequence N-X-(S or T); or
ii) a deletion of at least four consecutive amino acid residues, wherein the deletion includes at least residues corresponding to positions 249 and 250 of reference sequence SEQ ID NO: 1.

2. The modified coronavirus S protein of claim 1, wherein the modified coronavirus S protein comprising i) the substitution of one or more than one amino acid to introduce the N-glycosylation site, further comprises a deletion of one or more than one amino acids.

3. The modified coronavirus S protein of claim 1, wherein the deletion comprises:
i) at least amino acid residues corresponding to positions 247, 248, 249 and 250 of reference sequence SEQ ID NO: 1;
ii) at least amino acid residues corresponding to positions 248, 249, 250 and 251 of reference sequence SEQ ID NO: 1;
iii) at least amino acid residues corresponding to positions 249, 250, 251 and 252 of reference sequence SEQ ID NO: 1;
iv) at least amino acid residues corresponding to positions 246, 247, 248, 249 and 250 of reference sequence SEQ ID NO: 1;
v) at least amino acid residues corresponding to positions 247, 248, 249, 250 and 251 of reference sequence SEQ ID NO: 1;
vi) at least amino acid residues corresponding to positions 248, 249, 250, 251 and 252 of reference sequence SEQ ID NO: 1;
vii) at least amino acid residues corresponding to positions 246, 247, 248, 249, 250 and 251 of reference sequence SEQ ID NO: 1;
viii) at least amino acid residues corresponding to positions 247, 248, 249, 250, 251 and 252 of reference sequence SEQ ID NO: 1; or
ix) at least amino acid residues corresponding to positions 246, 247, 248, 249, 250, 251 and 252 of reference sequence SEQ ID NO: 1.

4. The modified coronavirus S protein of claim 2, wherein
i) the N-glycosylation site is introduced at the amino acid corresponding to position 251 of reference sequence SEQ ID NO: 1, and the deletion comprises at least amino acid residues corresponding to positions 249 and 250 of reference sequence SEQ ID NO: 1;
ii) the N-glycosylation site is introduced at the amino acid corresponding to position 252 of reference sequence SEQ ID NO: 1, and the deletion comprises at least amino acid residues corresponding to positions 249 and 250 of reference sequence SEQ ID NO: 1;
iii) the N-glycosylation site is introduced at the amino acid corresponding to position 253 of reference sequence SEQ ID NO: 1, and the deletion comprises at least amino acid residues corresponding to positions 249 and 250 of reference sequence SEQ ID NO: 1;
iv) the N-glycosylation site is introduced at the amino acid corresponding to position 253 of reference sequence SEQ ID NO: 1, and the deletion comprises at least four consecutive amino acid residues, wherein the deletion includes at least residues corresponding to positions 249 and 250 of reference sequence SEQ ID NO: 1;
v) the N-glycosylation site is introduced at the amino acid corresponding to position 253 of reference sequence SEQ ID NO: 1, and the deletion comprises at least amino acid residues corresponding to positions 246, 247, 248, 249, 250, 251 and 252 of reference sequence SEQ ID NO: 1.

5. The modified coronavirus S protein of claim 1, wherein the amino acid sequence modification comprises a substitution to an asparagine (N) at position corresponding to position 252 or 253 of reference sequence SEQ ID NO: 1 or the amino acid sequence modification comprises a substitution to an asparagine (N) at the position corresponding to position 251 and a substitution to a threonine (T) at position corresponding to position 253 of reference sequence SEQ ID NO: 1.

6. The modified coronavirus S protein of claim 1, wherein the modified coronavirus S protein comprises from 80% to 100% identity with the sequence of SEQ ID NO: 8, 10, 12, 16, 20, 39, 41, 43, 45, 47, 49, 51, 53, 55, or 57.

7. The modified coronavirus S protein of claim 1, wherein the modified coronavirus S protein is a chimeric S protein, wherein the chimeric S protein comprises a cytoplasmic tail derived from an influenza hemagglutinin.

8. The modified coronavirus S protein of claim 1, wherein the parent amino acid sequence is derived from Betacoronavirus.

9. The modified coronavirus S protein of claim 8, wherein the Betacoronavirus is from lineages A, B, C, or D of Betacoronavirus.

10. The modified coronavirus S protein of claim 1, wherein the modified coronavirus S protein comprises plant specific N-glycans.

11. A nucleic acid comprising a nucleotide sequence encoding the modified coronavirus S protein of claim 1.

12. A virus like particle (VLP) comprising the modified coronavirus S protein of claim 1.

13. (canceled)

14. (canceled)

15. A vaccine for inducing an immune response, the vaccine comprising an effective dose of the VLP of claim 12.

16. (canceled)

17. The vaccine of claim 13, wherein the vaccine is a multivalent vaccine, comprising a mixture of VLP.

18. A method for inducing immunity to a Coronavirus infection in a subject, the method comprising administering the vaccine of claim 13 to the subject.

19. (canceled)

20. (canceled)

21. A host or host cell comprising the VLP of claim 12.

22. (canceled)

23. (canceled)

24. A method of producing a virus like particle (VLP) in a host or host cell comprising:
a) introducing the nucleic acid of claim 11 into the host or host cell, or providing the host or host cell comprising the nucleic acid of claim 11, and
b) incubating the host or host cell under conditions that permit the expression of the nucleic acid, thereby producing the VLP and optionally extracting and purifying the VLP from the host or host cell.

25. (canceled)

26. A VLP produced by the method of claim 17.

27. A method of increasing production of a full-length coronavirus S protein in a host or host cell by modifying a parent coronavirus S protein, the method comprising:
a) introducing the nucleic acid of claim 11 into the host or host cell, or providing the host or host cell comprising the nucleic acid of claim 11, and
b) incubating the host or host cell under conditions that permit the expression of the nucleic acid, thereby producing a modified coronavirus S protein, wherein a higher amount or higher proportion of the modified coronavirus S protein is full-length modified coronavirus S protein compared to the parent coronavirus S protein produced under similar conditions in the host or host cell and optionally extracting and purifying the modified coronavirus S protein from the host or host cell.

28.-30. (canceled)

31. A method of modifying a coronavirus S protein to produce a modified coronavirus S protein with one or more than one amino acid sequence modification, wherein the one or more than one amino acid modification stabilize the modified coronavirus S protein, the method comprising:
i) introducing into the coronavirus S protein a substitution of one or more than one amino acid to introduce a N-glycosylation site at a position corresponding to positions 251, 252 or 253 of reference sequence SEQ ID NO: 1, wherein the N-glycosylation site is asparagine (N) in the consensus sequence N-X-(S or T); or
ii) introducing into the coronavirus S protein a deletion of at least four consecutive amino acid residues, wherein the deletion includes at least residues corresponding to positions 249 and 250 of reference sequence SEQ ID NO: 1, thereby modifying the coronavirus S protein.

32. (canceled)

33. (canceled)


SEQUENCE LISTING (57 sequences)

SEQ ID NO: 1 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3789 bp, synthetic construct
  source 1..3789: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 4 - protein, 1262 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 5 - DNA, 3768 bp, synthetic construct
  source 1..3768: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacgtgatcaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccacaactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatcagtatcggctgttccgcaagagtaaccttaagccctttgagagagatataagcactgaaatctaccaggctggcagtacgccctgtaatggcgtggaaggctttaattgttatagcccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcagggcgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgaacgtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 6 - protein, 1255 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 7 - DNA, 3768 bp, synthetic construct
  source 1..3768: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccacgactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 8 - protein, 1255 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 9 - DNA, 3789 bp, synthetic construct
  source 1..3789: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccggaaactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 10 - protein, 1262 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 11 - DNA, 3768 bp, synthetic construct
  source 1..3768: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccacaactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 12 - protein, 1255 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 13 - DNA, 3783 bp, synthetic construct
  source 1..3783: mol_type = other DNA
atggcgaaaaacgttgcgattttcggcttattgttttctcttcttgtgttggttccttctcagatcttcgcgtcccaatgcgtgaatcttagaacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttggacgtgtactatcataagaacaacaagtcttggatggaatctggcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatagatatcggctgttccgcaagagtaaccttaagccctttgagagagatataagcactgaaatctaccaggctggcagtaagccctgtaatggcgtggagggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcagggcgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccagaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccagaacgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 14 - protein, 1260 aa, synthetic construct
MAKNVAIFGLLFSLLVLVPSQIFASQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 15 - DNA, 3783 bp, synthetic construct
  source 1..3783: mol_type = other DNA
atggcgaaaaacgttgcgattttcggcttattgttttctcttcttgtgttggttccttctcagatcttcgcgtcccaatgcgtgaatcttagaacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttggacgtgtactatcataagaacaacaagtcttggatggaatctggcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccggaaactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatagatatcggctgttccgcaagagtaaccttaagccctttgagagagatataagcactgaaatctaccaggctggcagtaagccctgtaatggcgtggagggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcagggcgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccagaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccagaacgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 16 - protein, 1260 aa, synthetic construct
MAKNVAIFGLLFSLLVLVPSQIFASQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 17 - DNA, 3780 bp, synthetic construct
  source 1..3780: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgtgatttccggcactaacggtactaagaggttcgataatcctgtgctccctttcaatgacggcgtttactttgcaagcatcgagaagagtaacatcatccgaggttggatctttggcactaccctcgattcaaagacgcagagcctcctcattgtgaacaatgccactaacgtggtgatcaaagtttgcgagtttcagttctgcaatgaccctttcttggaccataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattatcgtgcgcgagcccgaggatcttccacaaggcttcagcgccctggaaccactcgtggacctcccaattggtatcaacatcactagatttcagactctgcttgccctccaccgatcctatctgacacccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcgacgaggttttcaatgctacacggtttgcctccgtgtatgcctggaaccgcaagagaatttccaattgcgtggccgattactccgtgctctacaatctggcaccctttttcacctttaagtgctatggcgtatcccctactaagcttaacgacttgtgtttcacaaacgtgtatgccgactcctttgtgatacggggcgacgaagttagacagatagcaccaggacagacgggaaacatagctgactacaactataagcttcctgatgacttcactggctgcgttatcgcgtggaattctaacaagctggactcaaaagtcagcggcaactataactatctctatcggctgttccgcaagagtaaccttaagccctttgagagagatataagcactgaaatctaccaggctggcaacaagccctgtaatggcgtggccggctttaattgttattttccactgagatcctatagctttagaccaacctacggcgtgggccaccaaccataccgcgtcgtggtgctctcctttgaactgctccacgctcccgcgactgtctgcggccccaagaagtccacgaaccttgtgaagaataagtgcgttaattttaatttcaacggcctcaagggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcagggcgtcaactgtactgaggtgcccgtagccatccatgcggatcagctcacaccaacttggagggtgtacagcaccggctccaatgtattccagactcgggccggatgccttattggcgccgaatacgtgaacaatagttacgaatgcgatattccaattggcgccggaatctgtgctagctaccagactcagacgaagtcccacggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaagagagcgctcacaggcatcgccgtggagcaggataagaatacccaggaggtgttcgcccaagttaagcagatctacaagaccccacccataaagtacttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaagggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccacaatgcccaggctctcaataccctcgtgaagcagctctcatctaagttcggcgcaatttccagtgtcctcaacgacatcttcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 18 - protein, 1259 aa, synthetic construct
MAKNVAIFGLLFSLLVLVPSQIFASQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 19 - DNA, 3780 bp, synthetic construct
  source 1..3780: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgtgatttccggcactaacggtactaagaggttcgataatcctgtgctccctttcaatgacggcgtttactttgcaagcatcgagaagagtaacatcatccgaggttggatctttggcactaccctcgattcaaagacgcagagcctcctcattgtgaacaatgccactaacgtggtgatcaaagtttgcgagtttcagttctgcaatgaccctttcttggaccataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattatcgtgcgcgagcccgaggatcttccacaaggcttcagcgccctggaaccactcgtggacctcccaattggtatcaacatcactagatttcagactctgcttgccctccaccgatcctatctgacacccggaaactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcgacgaggttttcaatgctacacggtttgcctccgtgtatgcctggaaccgcaagagaatttccaattgcgtggccgattactccgtgctctacaatctggcaccctttttcacctttaagtgctatggcgtatcccctactaagcttaacgacttgtgtttcacaaacgtgtatgccgactcctttgtgatacggggcgacgaagttagacagatagcaccaggacagacgggaaacatagctgactacaactataagcttcctgatgacttcactggctgcgttatcgcgtggaattctaacaagctggactcaaaagtcagcggcaactataactatctctatcggctgttccgcaagagtaaccttaagccctttgagagagatataagcactgaaatctaccaggctggcaacaagccctgtaatggcgtggccggctttaattgttattttccactgagatcctatagctttagaccaacctacggcgtgggccaccaaccataccgcgtcgtggtgctctcctttgaactgctccacgctcccgcgactgtctgcggccccaagaagtccacgaaccttgtgaagaataagtgcgttaattttaatttcaacggcctcaagggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcagggcgtcaactgtactgaggtgcccgtagccatccatgcggatcagctcacaccaacttggagggtgtacagcaccggctccaatgtattccagactcgggccggatgccttattggcgccgaatacgtgaacaatagttacgaatgcgatattccaattggcgccggaatctgtgctagctaccagactcagacgaagtcccacggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaagagagcgctcacaggcatcgccgtggagcaggataagaatacccaggaggtgttcgcccaagttaagcagatctacaagaccccacccataaagtacttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaagggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccacaatgcccaggctctcaataccctcgtgaagcagctctcatctaagttcggcgcaatttccagtgtcctcaacgacatcttcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 20 - protein, 1259 aa, synthetic construct
MAKNVAIFGLLFSLLVLVPSQIFASQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGNSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 21 - DNA, 4436 bp, synthetic construct
  source 1..4436: mol_type = other DNA
tggcaggatatattgtggtgtaaacaaattgacgcttagacaacttaataacacattgcggacgtttttaatgtactgaattaacgccgaatcccgggctggtatatttatatgttgtcaaataactcaaaaaccataaaagtttaagttagcaagtgtgtacatttttacttgaacaaaaatattcacctactactgttataaatcattattaaacattagagtaaagaaatatggatgataagaacaagagtagtgatattttgacaacaattttgttgcaacatttgagaaaattttgttgttctctcttttcattggtcaaaaacaatagagagagaaaaaggaagagggagaataaaaacataatgtgagtatgagagagaaagttgtacaaaagttgtaccaaaatagttgtacaaatatcattgaggaatttgacaaaagctacacaaataagggttaattgctgtaaataaataaggatgacgcattagagagatgtaccattagagaatttttggcaagtcattaaaaagaaagaataaattatttttaaaattaaaagttgagtcatttgattaaacatgtgattatttaatgaattgatgaaagagttggattaaagttgtattagtaattagaatttggtgtcaaatttaatttgacatttgatcttttcctatatattgccccatagagtcagttaactcatttttatatttcatagatcaaataagagaaataacggtatattaatccctccaaaaaaaaaaaacggtatatttactaaaaaatctaagccacgtaggaggataacaggatccccgtaggaggataacatccaatccaaccaatcacaacaatcctgatgagataacccactttaagcccacgcatctgtggcacatctacattatctaaatcacacattcttccacacatctgagccacacaaaaaccaatccacatctttatcacccattctataaaaaatcacactttgtgagtctacactttgattcccttcaaacacatacaaagagaagagactaattaattaattaatcatcttgagagaaaatggaacgagctatacaaggaaacgacgctagggaacaagctaacagtgaacgttgggatggaggatcaggaggtaccacttctcccttcaaacttcctgacgaaagtccgagttggactgagtggcggctacataacgatgagacgaattcgaatcaagataatccccttggtttcaaggaaagctggggtttcgggaaagttgtatttaagagatatctcagatacgacaggacggaagcttcactgcacagagtccttggatcttggacgggagattcggttaactatgcagcatctcgatttttcggtttcgaccagatcggatgtacctatagtattcggtttcgaggagttagtatcaccgtttctggagggtcgcgaactcttcagcatctctgtgagatggcaattcggtctaagcaagaactgctacagcttgccccaatcgaagtggaaagtaatgtatcaagaggatgccctgaaggtactcaaaccttcgaaaaagaaagcgagtaagttaaaatgcttcttcgtctcctatttataatatggtttgttattgttaattttgttcttgtagaagagcttaattaatcgttgttgttatgaaatactatttgtatgagatgaactggtgtaatgtaattcatttacataagtggagtcagaatcagaatgtttcctccataactaactagacatgaagacctgccgcgtacaattgtcttatatttgaacaactaaaattgaacatcttttgccacaactttataagtggttaatatagctcaaatatatggtcaagttcaatagattaataatggaaatatcagttatcgaaattcattaacaatcaacttaacgttattaactactaattttatatcatcccctttgataaatgatagtacaccaattaggaaggagcatgctcgcctaggagattgtcgtttcccgccttcagtttgcaagctgctctagccgtgtagccaatacgcaaaccgcctctccccgcgcgttgggaattactagcgcgtgtcgacaagcttgcatgccggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggcactccatttgaatctatcaaaccaaaacacattgagcaaaatggcgaaaaacgttgcgattttcggcttattgttttctcttcttgtgttggttccttctcagatcttcgcgacgtcactcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagacgtccagattttggcgatctattcaactgtcgccagttcattggtactggtagtctccctgggggcaatcagtttctggatgtgctctaatgggtctctacagtgtagaatatgtatttaaaggccttagtcgtgtcgtttttcaaataatataatccttttagggttttagttagtttaaattttctgttgctcctgtttagcaggtcgtgccttcagcaagcacacaaaaacagagtgtttattttaagttgtttgtttagtgattcaaaaaaaaaatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgcccacgtgactagtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 22 - DNA, 4985 bp, synthetic construct
  source 1..4985: mol_type = other DNA
gtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggcactccatttgaatctatcaaaccaaaacacattgagcaaaatggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaaaggccttagtcgtgtcgtttttcaaataatataatccttttagggttttagttagtttaaattttctgttgctcctgtttagcaggtcgtgccttcagcaagcacacaaaaacagagtgtttattttaagttgtttgtttagtgattcaaaaaaaaaatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 23 - DNA, 49 bp, synthetic construct
  source 1..49: mol_type = other DNA
tctcagatcttcgcgtcccaatgcgtgaatcttacgacgcgaacacagt

SEQ ID NO: 24 - DNA, 48 bp, synthetic construct
  source 1..48: mol_type = other DNA
acgacacgactaaggcctttaaatgcaaattctgcattgtaacgatcc

SEQ ID NO: 25 - protein, 1231 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 26 - protein, 1238 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 27 - protein, 1231 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHNSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 28 - protein, 1238 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPNDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 29 - protein, 1238 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTNGTSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 30 - protein, 1234 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 31 - protein, 1234 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 32 - protein, 1234 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 33 - protein, 1233 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 34 - protein, 1233 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 35 - protein, 1233 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 36 - protein, 1232 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 37 - protein, 1232 aa, synthetic construct
SQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 38 - DNA, 3789 bp, synthetic construct
  source 1..3789: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacacccaatgactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 39 - protein, 1262 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPNDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 40 - DNA, 3789 bp, synthetic construct
  source 1..3789: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatctgacaaatggaacctcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 41 - protein, 1262 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTNGTSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 42 - DNA, 3777 bp, synthetic construct
  source 1..3777: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgacccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 43 - protein, 1258 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 44 - DNA, 3777 bp, synthetic construct
  source 1..3777: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 45 - protein, 1258 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 46 - DNA, 3777 bp, synthetic construct
  source 1..3777: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatcctatgactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 47 - protein, 1258 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 48 - DNA, 3774 bp, synthetic construct
  source 1..3774: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccccggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 49 - protein, 1257 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 50 - DNA, 3774 bp, synthetic construct
  source 1..3774: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgaggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 51 - protein, 1257 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 52 - DNA, 3774 bp, synthetic construct
  source 1..3774: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgatccgactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 53 - protein, 1257 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 54 - DNA, 3771 bp, synthetic construct
  source 1..3771: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccacggagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 55 - protein, 1256 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI

SEQ ID NO: 56 - DNA, 3771 bp, synthetic construct
  source 1..3771: mol_type = other DNA
atggcgaaaaacgttgcgattttcggcttattgttttctcttcttgtgttggttccttctcagatcttcgcgtcccaatgcgtgaatcttacgacgcgaacacagttaccacccgcatatacaaatagcttcactcggggtgtttattaccccgacaaagtgttcaggtcctccgtgctccactcaacacaggacctctttcttcctttcttttctaacgtgacatggtttcatgccattcatgtatccggcactaacggtactaagaggttcgataatcctgtgctccctttcaatgacggcgtttactttgcaagcacagagaagagtaacatcatccgaggttggatctttggcactaccctcgattcaaagacgcagagcctcctcattgtgaacaatgccactaacgtggtgatcaaagtttgcgagtttcagttctgcaatgaccctttcttgggggtgtactatcataagaacaacaagtcttggatggaatctgaattccgcgtctatagcagcgccaacaactgcacctttgaatacgtgtcccagcccttccttatggacctggagggaaagcagggaaactttaagaatctgagagagttcgtgtttaaaaatatcgacggctattttaagatctattctaagcacacgcctattaatctcgtgcgcgatcttccacaaggcttcagcgccctggaaccactcgtggacctcccaattggtatcaacatcactagatttcagactctgcttgccctccaccgagactcctctagcggctggactgccggcgctgccgcttattacgttggttatcttcagccacgcacgttcctgctgaagtataacgagaatggtactattaccgatgccgtggattgtgcccttgaccccctgtccgaaactaagtgcacactcaagtcattcactgtggaaaaaggaatctaccagacaagcaattttcgggtccagcctactgagagcattgtgcgctttcctaacatcacaaatctttgccccttcggagaggttttcaatgctacacggtttgcctccgtgtatgcctggaaccgcaagagaatttccaattgcgtggccgattactccgtgctctacaatagtgcaagctttagcacctttaagtgctatggcgtatcccctactaagcttaacgacttgtgtttcacaaacgtgtatgccgactcctttgtgatacggggcgacgaagttagacagatagcaccaggacagacgggaaagatagctgactacaactataagcttcctgatgacttcactggctgcgttatcgcgtggaattctaacaacctggactcaaaagtcggcggcaactataactatctctatcggctgttccgcaagagtaaccttaagccctttgagagagatataagcactgaaatctaccaggctggcagtacgccctgtaatggcgtggaaggctttaattgttattttccactgcaatcctatggttttcagccaaccaatggcgtgggctaccaaccataccgcgtcgtggtgctctcctttgaactgctccacgctcccgcgactgtctgcggccccaagaagtccacgaaccttgtgaagaataagtgcgttaattttaatttcaacggcctcactggaacaggagtgctcactgagagtaacaagaagttcctgccatttcaacaatttggcagagacatagccgatactactgacgccgttagggacccccagaccctcgagattctcgatataacgccctgctccttcggtggagtttccgtgatcacgccaggcaccaataccagtaaccaggtcgccgtgctgtatcaggatgtcaactgtactgaggtgcccgtagccatccatgcggatcagctcacaccaacttggagggtgtacagcaccggctccaatgtattccagactcgggccggatgccttattggcgccgaacacgtgaacaatagttacgaatgcgatattccaattggcgccggaatctgtgctagctaccagactcagacgaactccccaggcagcgccagcagcgttgccagccagtcaatcatcgcttatacaatgtcacttggagccgaaaactccgtggcttactcaaacaacagcatcgccatccccacaaacttcaccatatccgtgacaactgagattctgccagtgtccatgactaagacgtccgtagattgcactatgtacatatgcggcgacagcacagaatgttctaatctgctgctgcaatatggaagcttctgcactcaactgaacagagcgctcacaggcatcgccgtggagcaggataagaatacccaggaggtgttcgcccaagttaagcagatctacaagaccccacccataaaggatttcggtggattcaattttagtcagatactcccagacccatctaagccatccaagaggagctttatcgaggatcttttgtttaacaaagttactctggccgacgccggtttcatcaagcagtacggagattgcctcggcgacatcgctgctcgtgacctcatctgtgcgcaaaagtttaacggtctgacggtgctgcctcccctccttactgatgaaatgatcgcccagtataccagcgcactcctcgctggcaccataacatccggttggacattcggcgctggtgcagcactgcagataccattcgccatgcaaatggcatatcgtttcaacggtatcggtgtcacacagaatgtcctatatgagaaccagaagctgatcgcaaatcagttcaatagtgccatcggaaaaatccaggatagccttagcagcacagcctcagcccttggcaaactccaggatgtcgtgaaccagaatgcccaggctctcaataccctcgtgaagcagctctcatctaatttcggcgcaatttccagtgtcctcaacgacatcctcagccgcctcgacccccccgaggccgaagtgcagattgacagactgattacaggtcgactccagagcctccagacttacgtgactcagcagctgataagagccgccgagataagggccagcgctaacctggctgccacaaagatgtctgagtgcgtgctgggccagtccaagagagtagacttctgtggcaaaggctaccatctgatgagcttcccacaatccgcacctcacggcgtagtgttcctccacgtgacatatgtaccggctcaggagaagaatttcactaccgctcctgctatatgccatgatggaaaggctcacttcccccgggagggggtgttcgtgtccaacggcacccattggtttgtgactcagcggaatttctacgaaccccagatcataaccactgacaacacatttgtgtccggaaattgtgacgtggtcattggaatagtgaacaacactgtttatgatccactgcagccagaacttgacagctttaaggaggagctcgacaagtacttcaagaatcatacgtcaccagatgtggacctcggagatattagcggtatcaatgccagtgttgtcaatattcagaaggaaatagaccgccttaatgaggtcgccaaaaatctgaacgagagcctcatcgatcttcaggagctgggcaaatatgagcagtacatcaagtggccttggtatatttggcttggcttcatcgccggcctgatcgccatagtaatggtcacaattatgctctctttatggatgtgctccaatggatcgttacaatgcagaatttgcatttaa

SEQ ID NO: 57 - protein, 1256 aa, synthetic construct
MAKNVAIFGLLFSLLVLVPSQIFASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLSLWMCSNGSLQCRICI